SEQUENCE LISTING (9 sequences)

SEQ ID NO: 1 - DNA, 15 bp, Artificial Sequence
  Description of Artificial Sequence Synthetic oligonucleotide
acatgagcttacaac

SEQ ID NO: 2 - DNA, 12 bp, Artificial Sequence
  Description of Artificial Sequence Synthetic oligonucleotide
tgcaaaagaaca

SEQ ID NO: 3 - DNA, 12 bp, Artificial Sequence
  Description of Artificial Sequence Synthetic oligonucleotide
acgtnttnttnt

SEQ ID NO: 4 - DNA, 15 bp, Artificial Sequence
  Description of Artificial Sequence Synthetic oligonucleotide
atgcaaaagaacatt

SEQ ID NO: 5 - DNA, 12 bp, Artificial Sequence
  Description of Artificial Sequence Synthetic oligonucleotide
tgcagaaaaata

SEQ ID NO: 6 - DNA, 12 bp, Artificial Sequence
  Description of Artificial Sequence Synthetic oligonucleotide
tgcacaataaga

SEQ ID NO: 7 - DNA, 16 bp, Artificial Sequence
  Description of Artificial Sequence Synthetic oligonucleotide
tgcacaataagaacga

SEQ ID NO: 8 - DNA, 12 bp, Artificial Sequence
  Description of Artificial Sequence Synthetic oligonucleotide
cggantcnatng

SEQ ID NO: 9 - DNA, 12 bp, Artificial Sequence
  Description of Artificial Sequence Synthetic oligonucleotide
gtcancgnaanc

(19) United States
(12) Patent Application Publication
(10) Pub. No.: US 2006/0073501 A1
(43) Pub. Date: Apr. 6, 2006

(54) Methods for long-range sequence analysis of nucleic acids

(76) Inventors: Dirk Johannes Van Den Boom, La Jolla, CA (US); Sebastian Boecker, Bielefeld (DE)

Correspondence Address: BIOTECHNOLOGY LAW GROUP, c/o PORTFOLIO IP, P.O. BOX 52050, MINNEAPOLIS, MN, 55402, US

(21) Appl. No.: 11/222,991

(22) Filed: Sep. 8, 2005

Related U.S. Application Data
(60) Provisional application No. 60/608,712, filed on Sep. 10, 2004.

(51) Int. Cl. C12Q 1/68

(52) U.S. Cl. 435/6

(57) ABSTRACT

Provided are methods for sequencing a target nucleic acid by fragmenting a target nucleic acid, hybridizing fragments to an array of capture oligonucleotides, determining the mass of the hybridized fragments, and constructing a nucleotide sequence of the target nucleic acid from the mass measurements.

### RELATED APPLICATIONS

This application claims the benefit of 60/608,712 filed Sep. 10, 2004, which is related to U.S. application Ser. No. 10/412,801 Lin et al., filed Apr. 11, 2003, entitled “METHOD AND DEVICE FOR PERFORMING CHEMICAL REACTION ON A SOLID SUPPORT;” U.S. provisional application Ser. No. 60/457,847 to Lin et al., filed Mar. 24, 2003, entitled “METHOD AND DEVICE FOR PERFORMING CHEMICAL REACTION ON A SOLID SUPPORT;” U.S. provisional application Ser. No. 60/372,711 to Lin et al., filed Apr. 11, 2002, entitled “METHOD AND DEVICE FOR PERFORMING CHEMICAL REACTION ON A SOLID SUPPORT;” U.S. application Ser. No. 10/723,365 to van den Boom et al., filed Nov. 27, 2003, entitled “FRAGMENTATION-BASED METHODS AND SYSTEMS FOR SEQUENCE VARIATION DETECTION AND DISCOVERY;” U.S. provisional application Ser. No. 60/429,895 to van den Boom et al., filed Nov. 27, 2002, entitled “FRAGMENTATION-BASED METHODS AND SYSTEMS FOR SEQUENCE VARIATION DETECTION AND DISCOVERY;” to U.S. provisional Ser. No. 10/830,943 to Bocker et al., filed Apr. 22, 2004, entitled “FRAGMENTATION-BASED METHODS AND SYSTEMS FOR DE NOVO SEQUENCING;” and to U.S. provisional Ser. No. 60/466,006 to Bocker et al., filed Apr. 25, 2003, entitled “FRAGMENTATION-BASED METHODS AND SYSTEMS FOR DE NOVO SEQUENCING.” The subject matter and content of each of these non-provisional and provisional applications is incorporated by reference in its entirety.

### FIELD OF THE INVENTION

Methods for nucleic acid analysis are provided.

### BACKGROUND

The analysis of the structure of various biopolymers is an area of great importance in medicine and research. Molecular genetics depends on a knowledge of the nucleotide sequence of DNA or RNA molecules. The amino acid sequence of proteins provides information useful for studying protein function and regulation. Various strategies exist for analyzing the sequence of biopolymers. The most commonly used method of determining the sequence of nucleic acids, the dideoxy method, involves creating four sets of sub-sequences of a DNA molecule that terminate at each of the four bases, separating the fragments by polyacrylamide gel electrophoresis (PAGE), and reading the resultant bands to determine the sequence. Gel electrophoresis can be slow and subject to errors.

A method that has been proposed to overcome drawbacks of sequencing by gel electrophoresis is a method termed sequencing by hybridization, see, e.g., Bains and Smith, J. Theoret. Biol., 135:303-307 (1998); Lysov et al., Dokl. Acad. Sci. USSR 303:1508-1511 (1988); Drmanac et al., Genomics 4:114-128 (1989); Pevzner, J. Biomolec. Struct. Dynamics 7(1):63-73 (1989); Pevzner and Lipschutz, Nineteenth Symp. on Math. Found. of Comp. Sci., LNCS-841: 143-258 (1994); Waterman, Introduction to Computational Biology, Chapman and Hall, London, 1995. Sequencing by hybridization (SBH) is a DNA sequencing technique in which an array (SBH chip) of short sequences of nucleotides (probes) is brought in contact with a solution of (replicas of) the target DNA sequence. A biochemical method determines the subset of probes that bind to the target sequence (the spectrum of the sequence), and a combinatorial method is used to reconstruct the DNA sequence from the spectrum. As technology limits the number of probes on the SBH chip, a challenging combinatorial question is the design of the smallest set of probes that can sequence an arbitrary random DNA string of a given length.

Implementations of SBH use “classical” probing schemes, i.e., chips accommodating all 4^{k} k-mer oligonucleotides (“solid” probes with no gaps), the symbols being the well-known DNA bases {A, C, G, T} and k being a technology-dependent integer parameter. It has been said that “[t]he main challenge for sequencing by hybridization is to reliably detect the perfect duplexes and discriminate them from duplexes containing mismatched base pairs” (Chechetkin et al., J. of Biomolecular Structure & Dynamics 18(1):83-101 (2000)). Thus, sequencing by hybridization methods attempt to avoid and minimize mismatched base pairing, which results in false-positive or false-negative results, ultimately resulting in failed sequencing methods.

The SBH methods rely on the avoidance of mismatch hybridization to eliminate false-positive and/or false-negative readings. Therefore, there is a need for hybridization-based methods of obtaining de novo nucleic acid sequence information that permits mismatch hybridization. Thus, among the objects herein, it is an object to provide methods of obtaining de novo nucleic acid sequence information that permits mismatch hybridization.

### SUMMARY

Among the methods provided herein are methods for obtaining de novo nucleic acid sequence information that permits mismatch hybridization. Provided herein are methods for sequence analysis of nucleic acids (including de novo sequencing), comprising generating overlapping fragments of a target nucleic acid; hybridizing the fragments to an array of capture oligonucleotides on a solid support under conditions that do not eliminate mismatched hybridization to form an array of captured fragments; determining the mass of the captured fragments at each locus in the array by determining the mass thereof, such as by mass spectrometric analysis; and constructing a nucleotide sequence or a set of nucleotide sequences of the target nucleic acid from a set of mass signals acquired from each array position. Also provided herein are methods for sequencing nucleic acids, comprising generating overlapping fragments of a target nucleic acid; hybridizing the fragments to an array of capture oligonucleotides on a solid support to form an array of captured fragments, wherein at least a subset of the capture oligonucleotides are partially degenerate; determining the mass of the captured fragments at each locus in the array by determining the mass(es) thereof, such as by mass spectrometric analysis; and constructing a nucleotide sequence or a set of nucleotide sequences of the target nucleic acid from a set of mass signals acquired from each array position. In one embodiment, the overlapping fragments are randomly generated.

The sequence information obtained from the samples using the methods provided herein can be used for genotyping and haplotyping, multiplexed genotyping and haplotyping, nucleic acid mixture analysis, long-range resequencing, long-range detection of sequence variation and mutations, multiplex sequencing, long-range methylation pattern analysis, organism identification, pathogen identification and typing, among others.

Thus, the methods provided herein advantageously merge solid phase hybridization-based methodology with algorithm-based compositional analysis of the hybridized products to significantly enhance solid-phase hybridization-based sequence analysis using mass spectrometry. One advantage of the methods provided herein is the significantly increased quantity and accuracy of target nucleic acid sequence read length that can be achieved compared to previous methods. The higher (long-range) sequence read length is accomplished using mass spectrometric analysis of non-specifically cleaved or partially specifically-cleaved target nucleic acids subsequently bound to a solid-phase to capture oligonucleotides, some or all of which can be partially degenerate. For example, the methods provided herein are able to sequence in one reaction/experiment at least 250, 500, 600, 700, 800, 900, 1,000, 1,500, 2,000, 3,000, 4,000, 5,000, 6,000, 7,000, 8,000, 9,000 up to 10,000 or more nucleotides. To accomplish this, the fragments generated for analysis by the methods provided herein are ultimately ordered to provide the sequence of the larger target nucleic acid.

In another embodiment, a multiplicity of shorter target nucleic acid fragments of shorter lengths are sequenced or analyzed by the methods provided herein. These multiplexed shorter sequence sets are useful, for example, in re-sequencing methods when part of the part of a particular sequence is known. These multiplexed shorter sequence sets also are useful for multiplexed genotyping, haplotyping, SNP and methylation detection methods.

The fragments can be generated by total or partial non-specific cleavage and/or by partial specific cleavage, and typically overlapping fragments are obtained for analysis. The overlapping fragments can be obtained using a single non-specific cleavage reaction and/or complementary or partial base-specific cleavage reactions such that alternative overlapping fragments of the same target biomolecule sequence are obtained. The cleavage means can be enzymatic, chemical, physical or a combination thereof, and typically, overlapping fragments are generated. Accordingly, depending on the particular method selected for generating the overlapping fragments, such overlapping fragments may or may not be randomly generated.

The masses of the cleaved and uncleaved target sequence fragments can be determined using methods known in the art including but not limited to mass spectrometry and gel electrophoresis. In a typical embodiment, MALDI-TOF mass spectrometry is used to determine the masses of the fragments. Chips and kits for performing high-throughput mass spectrometric analyses are commercially available from SEQUENOM, INC. under the trademark MassARRAY7. Another exemplary chip for use herein is the “h-chip” set forth in related U.S. application Ser. Nos. 60/372,711, filed Apr. 11, 2002, 60/457,847, filed Mar. 24, 2003, and Ser. No. 10/412,801, filed Apr. 11, 2003, incorporated herein by reference, in its entirety.

Accordingly, in one embodiment, the methods provided herein combine the high throughput capabilities of solid-phase hybridization with mass spectrometry detection and identification of the overlapping cleavage products that are sorted on the solid-phase. The methods provided herein also improve accuracy and clarity of identification of fragment signals produced by non-specific fragmentation or partial specific-fragmentation, and also increase in speed of analysis of these signals by using algorithms that reconstruct the sequences within either one target nucleic acid or a set of target nucleic acids.

### BRIEF DESCRIPTION OF THE DRAWINGS

FIG. 1 depicts the generation of overlapping fragments.

FIG. 2 shows multiple fragments hybridizing to the degenerate capture oligonucleotides on a solid-support.

FIG. 3 depicts the “trimming” of the hybridized capture oligonucleotide:target fragment duplex.

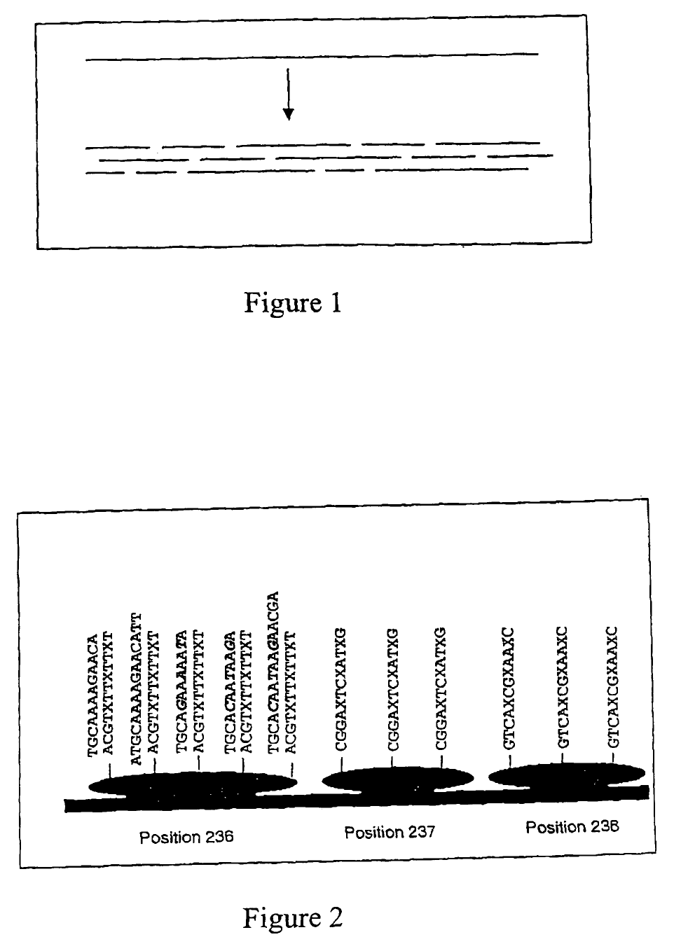

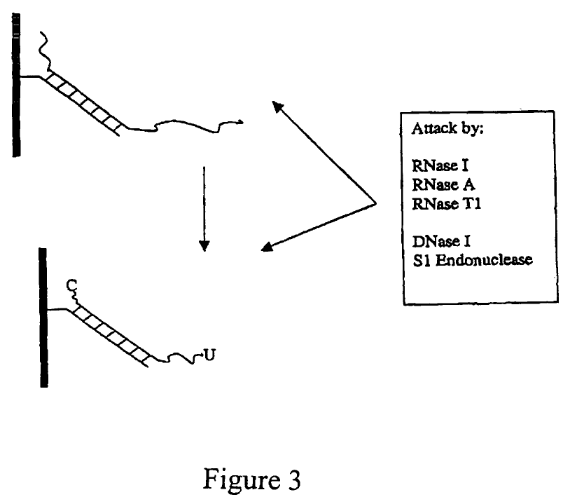

### DETAILED DESCRIPTION

A. Definitions
B. Methods for Sequencing Nucleic Acid Molecules
C. Target Nucleic Acid Molecules
1. Sources
   2. Preparation
   3. Size and Composition of Target Nucleic Acid Molecule
   4. Amplification
D. Fragmentation
   1. Enzymatic Fragmentation of Polynucleotides
      a. Endonuclease Fragmentation of Polynucleotides
      b. Nuclease Fragmentation
      C. Nucleic Acid Enzyme Fragmentation
      d. Base-Specific Fragmentation
   2. Physical Fragmentation of Polynucleotides
   3. Chemical Fragmentation of Polynucleotides
   4. Combination of Fragmentation
   5. Fragmentation After Hybridization
E. Capture Oligonucleotides
   1. Controlling Complexity of Target Nucleic Acid Fragments
      a. Methods of Controlling Complexity
      b. Regions of a Fragment
      c. Partially Single-Stranded Capture Oligonucleotide
   2. Composition of Capture Oligonucleotides
      a. Types of Nucleotides
         i. Universal Bases
         ii. Semi-Universal Bases
      b. Other Characteristics
      c. Making the Capture Oligonucleotides
F. Solid Supports and Arrays
G. Specific or Non-Specific Hybridization
H. Trimming
I. Information Relating to the Target Nucleic Acid Fragments
   1. Molecular Mass
      a. Mass Spectrometric Analysis
      b. Other Measurement Methods
   2. Mass Peak Characteristics
   3. Capture Oligonucleotide and Hybridization Conditions
   4. Fragmentation Conditions
J. Nucleotide Sequence Construction
K. Identifying a Nucleotide Sequence by Mass Pattern
L. Identifying a Portion of a Target Nucleic Acid
M. Applications
   1. Long Range Resequencing
   2. Long Range Detection of Mutations/Sequence Variations
   3. Multiplex Sequencing
   4. Long Range Methylation Pattern Analysis
   5. Organism Identification
   6. Pathogen Identification and Typing
   7. Molecular Breeding and Directed Evolution
   8. Target Nucleic Acid Fragments as Markers
   9. Detecting the presence of viral or bacterial nucleic acid sequences indicative of an infection
   10. Antibiotic Profiling
   11. Identifying disease markers
   12. Haplotyping
   13. DNA Repeats
   14. Detecting Allelic Variation
   15. Determining Allelic Frequency
   16. Epigenetics
Examples
   A. Definitions

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, “array” refers to a collection of elements, such as nucleic acids. Typically an array contains three or more members. An addressable array is one in which the members of the array are identifiable, such as by position on a solid support. Hence, members of the array can be immobilized at discrete identifiable loci on the surface of a solid phase or otherwise identifiable, such as by attaching or labeling with tags, including electronic and chemical tags. Arrays include, but are not limited to, a collection of elements on a single solid phase surface, such as a collection of oligonucleotides on a chip.

As used herein, “specifically hybridizes” refers to hybridization of a probe or primer only to a target sequence preferentially to a non-target sequence, typically under high stringency hybridization conditions. For example, specific hybridization includes the hybridization of a probe to a target sequence that is 100% complementary to the probe. Those of skill in the art are familiar with parameters that affect hybridization; such as temperature, probe or primer length and composition, buffer composition and salt concentration and can readily adjust these parameters to achieve specific hybridization of a nucleic acid to a target sequence.

As used herein: stringency of hybridization refers to the washing conditions for removing the non-specific binding of capture oligonucleotides to target nucleic acid fragments. Exemplary conditions for hybridization are as follows:
1) high stringency: 0.1×SSPE, 0.1% SDS, 65 EC
2) medium stringency: 0.2×SSPE, 0.1% SDS, 50 EC
3) low stringency: 1.0×SSPE, 0.1% SDS, 50 EC

Those of skill in this art know that the washing step selects for stable hybrids and also know the ingredients of SSPE (see, e.g., Sambrook, E. F. Fritsch, T. Maniatis, in: Molecular Cloning, A Laboratory Manual, Cold Spring Harbor Laboratory Press (1989), vol. 3, p. B.13, see, also, numerous catalogs that describe commonly used laboratory solutions). SSPE is pH 7.4 phosphate-buffered 0.18 M NaCl. Further, those of skill in the art recognize that the stability of hybrids is determined by Tₘ, which is a function of the sodium ion concentration and temperature (Tₘ=81.5 EC-16.6(log₁₀[Na⁺])+0.41 (% G+C)−600/1)), so that the parameters in the wash conditions important to hybrid stability are sodium ion concentration in the SSPE (or SSC) and temperature. Specific hybridization typically occurs under conditions of high stringency. It is understood that equivalent stringencies can be achieved using alternative buffers, salts and temperatures.

As used herein “nucleic acid” or “nucleic acid molecule” refers to polynucleotides such as deoxyribonucleic acid (DNA) and ribonucleic acid (RNA). The term should also be understood to include, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, “mass spectrometry” encompasses any suitable mass spectrometric format known to those of skill in the art. Such formats include, but are not limited to, Matrix-Assisted Laser Desorption/Ionization, Time-of-Flight (MALDI-TOF), Electrospray (ES), IR-MALDI (see, e.g., published International PCT application No. 99/57318 and U.S. Pat. No. 5,118,937), Orthogonal-TOF (O-TOF), Axial-TOF (A-TOF), Linear/Reflectron (RETOF), Ion Cyclotron Resonance (ICR), Fourier Transform and combinations thereof. MALDI, particularly UV and IR, are among the formats known in the art. See also, Aebersold and Mann, Mar. 13, 2003, Nature, 422:198-207 (e.g., at FIG. 2) for a review of exemplary methods for mass spectrometry suitable for use in the methods provided herein, which is incorporated herein in its entirety by reference. MALDI methods typically include UV-MALDI or IR-MALDI.

As used herein, the phrase “mass spectrometric analysis” refers to the determination of the charge to mass ratio of atoms, molecules or molecule fragments.

As used herein, mass spectrum refers to the presentation of data obtained from analyzing a biopolymer or fragment thereof by mass spectrometry either graphically or encoded numerically or otherwise presented.

As used herein, pattern with reference to a mass spectrum or mass spectrometric analyses, refers to a characteristic distribution and number of signals, peaks or digital representations thereof.

As used herein, signal, peak, or measurement, in the context of a mass spectrum and analysis thereof refers to the output data, which can reflect the charge to mass ratio of an atom, molecule or fragment of a molecule, and also can reflect the amount of the atom, molecule, or fragment thereof, present. The charge to mass ratio can be used to determine the mass of the atom, molecule or fragment of a molecule, and the amount can be used in quantitative or semi-quantitative methods. For example, in some embodiments, a signal peak or measurement can reflect the number or relative number of molecules having a particular charge to mass ratio. Signals or peaks include visual, graphic and digital representations of output data.

As used herein, intensity, when referring to a measured mass, refers to a reflection of the relative amount of an analyte present in the sample or composition compared to other sample or composition components. For example, an intensity of a first mass spectrometric peak or signal can be reported relative to a second peak of a mass spectrum, or can be reported relative to the sum of all intensities of peaks. One skilled in the art can recognize a variety of manners of reporting the relative intensity of a peak. Intensity can be represented as the peak height, peak width at half height, area under the peak, signal to noise ratio, or other representations known in the art.

As used herein, comparing measured masses or mass peaks refers to analyzing one or more measured sample mass peaks to one or more sample or reference mass peaks. For example, measured sample mass peaks can be analyzed by comparison with a calculated mass peak pattern, and any overlap between measured mass peaks and calculated mass peaks can be determined to identify the sample mass or molecule. A reference mass peak is a representation of the mass of a reference atom, molecule or fragment of a molecule.

As used herein, a reference mass is a mass with which a measured sample mass can be compared. A comparison of a sample mass with a reference mass can identify a sample mass as the same as or different from the reference mass. Such a reference mass can be calculated, can be present in a database or can be experimentally determined. A calculated reference mass can be based on the predicted mass of a nucleic acid. For example, calculated reference masses can be based on a predicted fragmentation pattern of a target nucleic acid molecule of known or predicted sequence. An experimentally derived reference mass can arise from a measured mass of any nucleic acid sample. For example, experimentally derived masses can be masses measured after treating nucleic acid molecule under fragmentation conditions and contacting the fragments with capture oligonucleotides. A database of reference masses can contain one or more reference masses where the reference masses can be calculated or experimentally determined; a database can contain reference masses corresponding to the calculated or experimentally determined fragmentation pattern of a target nucleic acid molecule; a database can contain reference masses corresponding to the calculated or experimentally determined fragmentation patterns of two or more target nucleic acid molecules.

As used herein, a reference nucleic acid molecule refers to a nucleic acid molecule of known nucleotide sequence or known identity (e.g., a locus without known sequence, but with known correlation to a disease). A reference nucleic acid can be used to calculate or experimentally derive reference masses. A reference nucleic acid used to calculate reference masses is typically a nucleic acid containing a known nucleotide sequence. A reference nucleic acid used to experimentally derive reference masses can have, but is not required to have, a known sequence; methods such as those disclosed herein or otherwise known in the art can be used to identify the nucleotide sequence of a reference nucleic acid even when the reference nucleic acid does not have a known sequence.

As used herein, a correlation between one or more sample masses (or one or more sample mass peak characteristics) and one or more reference masses (or one or more reference mass peak characteristics), and grammatical variants thereof, refers to a comparison between or among one or more sample masses (or one or more sample mass peak characteristics) and one or more reference masses (or one or more reference mass peak characteristics), where an increasing similarity of masses is indicative of an increasing likelihood that the nucleotide sequence of the target nucleic acid molecule or fragment thereof is that same as the nucleotide sequence of the reference nucleic acid.

As used herein, a correlation between one or more sample mass peaks and one or more reference mass peaks, and grammatical variants thereof, refers to the relation between one or more sample mass peaks and one or more reference mass peaks, where an increasing similarity in one or more mass peak characteristics between the one or more sample mass peaks and the one or more reference mass peaks is indicative of an increasing likelihood that at least a portion of the sample target nucleic acid is the same as at least a portion of the reference nucleic acid, or an increasing likelihood that the nucleotide sequence at one or more nucleotide positions of the target nucleic acid is the same as the nucleotide sequence at one or more nucleotide positions of the reference nucleic acid.

As used herein, a correlation between a target nucleic acid molecule nucleotide sequence and a reference nucleotide sequence, refers to a similarity or identity of the nucleotide sequence of a target nucleic acid molecule to that of a reference.

As used herein, “analysis” refers to the determination of particular properties of a single oligonucleotide, or of mixtures of oligonucleotides. These properties include, but are not limited to, the nucleotide composition and complete sequence of an oligonucleotide or of mixtures of oligonucleotides, the existence of single nucleotide polymorphisms and other mutations between more than one oligonucleotide, the masses and the lengths of oligonucleotides and the presence of a molecule or sequence within molecule in a sample.

As used herein, “multiplexing,” “multiplexed,” “a multiplexed reaction,” or grammatical variations thereof, refers to the simultaneous assessment or analysis of more than one molecule, such as a biomolecule (e.g., an oligonucleotide molecule) in a single reaction or in a single mass spectrometric or other sequence measurement, i.e., a single mass spectrum or other method of reading sequence.

As used herein, amplifying refers to means for increasing the amount of a biopolymer, especially nucleic acids. Based on the 5′ and 3′ primers that are chosen, amplification also serves to restrict and define the region of the genome which is subject to analysis. Amplification can be by any means known to those skilled in the art, including use of the polymerase chain reaction (PCR) etc. Amplification, e.g., PCR must be done quantitatively when the frequency of polymorphism is required to be determined.

As used herein, the phrase “statistically range in size” refers to the size range for a majority of the fragments generated using partial cleavage, such that some of the fragments may be substantially smaller or larger than most of the other fragments within the particular size range. For example, the statistical size range of 12-30 bases can also include some oligonucleotides as small as 1 nucleotide or as large as 300 nucleotides or more, but these particular sizes statistically occur relatively rarely. A statistical range of fragments can include where 60% of the fragments are within the desired size range, where 60% or more of the fragments are within the desired size range, where 70% or more of the fragments are within the desired size range, where 80% or more of the fragments are within the desired size range, where 90% or more of the fragments are within the desired size range, or where 95% or more of the fragments are within the desired size range.

As used herein, the phrase “hybridizing”, or grammatical variations thereof, refers to binding of a nucleic acid sequence to its complete or partial complementary strand. The term hybridizing, as used herein, can apply both to the binding of perfectly complementary strands, and also to the binding of strands that are not perfectly complementary. Thus, hybridizing can include instances where a first nucleic acid binds to a second nucleic acid, where the first and second nucleic acids have one or more mismatched bases.

As used herein, the phrase “under conditions that do no eliminate mismatched hybridization” refers to hybridization conditions that permit the binding of capture oligonucleotides having 1 or more base pair mismatches. In some embodiments, the number of mismatches permitted is selected from no more than 5, no more than 4, no more than 3, no more than 2, and no more than 1 base pair mismatch.

As used herein, the phrase “captured fragments” refers to target nucleic acid fragments that are bound to capture oligonucleotides, for example, capture oligonucleotides on a solid-phase.

As used herein, “degenerate position” refers to a location on a nucleotide that contains, in place of one of the four typically occurring bases, a substituent that binds to more than one nucleotide. For example, a degenerate position on a nucleotide can be a nucleotide position containing a universal base or a semi-universal base. A partially degenerate nucleotide refers to nucleotide that contains at least one degenerate position and at least one non-degenerate position (e.g., contains a universal or semi-universal base and a non-degenerate base such as A, G, C or T[U), or to a nucleotide that contains at least one degenerate position that preferentially binds some nucleotides relative to other nucleotides (e.g., contains at least one semi-universal base). In certain embodiments herein, the partially degenerate oligonucleotides contain at least 10%, 20%, 30%, 40%, up to 50% degenerate positions. For example, for capture oligonucleotides having a length of 20 nucleotides, these partially degenerate oligonucleotides can contain 1, 2, 3, 4, 5, 6, 7, 8, 9 up to 10 degenerate positions. In other embodiments, a degenerate oligonucleotide can contain more than 50% degenerate positions, including 100% degenerate positions. For example, an oligonucleotide having a length of 20 nucleotides can contain 20 semi-universal nucleotides, or 10 universal nucleotides and 10 semi-universal nucleotides.

As used herein, solid support particles refers to materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and can be on the order of cubic microns; typically the particles have a diameter of more than about 1.5 microns and less than about 15 microns, such as about 4-6 microns. Such particles are collectively called “beads.”

As used herein, “solid support” refers to an insoluble support that can provide a surface on which or over which a reaction can be conducted and/or a reaction product can be retained at identifiable loci. Support can be fabricated from virtually any insoluble or solid material. For example, silica gel, glass (e.g., controlled-pore glass (CPG)), nylon, Wang resin, Merrifield resin, Sephadex, Sepharose, cellulose, a metal surface (e.g., steel, gold, silver, aluminum, and copper), silicon, and plastic material (e.g., polyethylene, polypropylene, polyamide, polyester, polyvinylidenedifluoride (PVDF)). Exemplary solid supports include, but are not limited to flat supports such as glass fiber filters, glass surfaces, metal surfaces (steel, gold, silver, aluminum, copper and silicon), and plastic materials. The solid support is in any desired form suitable for mounting on the cartridge base, including, but not limited to: a plate, membrane, wafer, a wafer with pits, a porous three-dimensional support, and other geometries and forms known to those of skill in the art. Exemplary support are flat surfaces designed to receive or link samples at discrete loci, such as flat surfaces with hydrophobic regions surrounding hydrophilic loci for receiving, containing or binding a sample.

As used herein, the phrases “non-specifically cleaved” or “non-specific fragmentation”, in the context of nucleic acid fragmentation, refers to the fragmentation of a target nucleic acid molecule at random locations throughout, such that various fragments of different size and nucleotide sequence content are randomly generated. Fragmentation at random locations, as used herein, does not require absolute mathematical randomness, but instead only a lack of strong sequence-based preference in fragmentation. For example, fragmentation by irradiative or shearing means can cleave DNA at nearly any position; however, such methods may result in fragmentation at some locations with slightly more frequently than other locations. Nevertheless, fragmentation at nearly all positions with only a slight sequence preference are considered random for purposes herein. Non-specific cleavage using the methods described herein result in the generation of overlapping nucleotide fragments.

As used herein, the terms partial or incomplete cleavage, or partial or incomplete fragmentation, or grammatical variations thereof, refer to a reaction in which only a fraction of the respective cleavage sites for a particular fragmentation conditions are actually cleaved. The fragmentation conditions can be, but are not limited to presence of an enzyme, a chemical, or physical force. As set forth herein, one way of achieving partial fragmentation is by using a mixture of cleavable or non-cleavable nucleotides or amino acids during target biomolecule production, such that the particular cleavage site contains uncleavable nucleotides or amino acids, which renders the target biomolecule partially cleaved, even when the cleavage reaction is run to completion. For example, if an uncleaved target biomolecule has 4 potential cleavage sites (e.g., cut bases for a nucleic acid) therein, then the resulting mixture of products from partial cleavage can have any combination of fragments of the target biomolecule resulting from: a single cleavage at a first, second, third or fourth cleavage site; double cleavage at any one or more combinations of 2 cleavage sites; or triple cleavage at any one or more combinations of 3 cleavage sites. Products from partial cleavage can be present in the same mixture as products from total cleavage.

As used herein, the phrase “overlapping fragments” refers to fragments that have one or more nucleotide positions from the native target nucleic acid in common. As used herein, “statistically overlapping fragments” refers to a group of fragments where a subpopulation of defined size overlaps with at least one other fragment. For example, statistically overlapping fragments can refer to a group of fragments wherein at least 50%, at least 60%, at least 70%, at least 80%, at least 85%, at least 90%, at least 95% or at least 98% of the fragments overlap with at least one other fragment.

As used herein, “a non-specific RNase” refers to an enzyme that cleaves a RNA molecule irrespective of the nucleotide sequence at the cleavage site. An exemplary non-specific RNase is RNase I.

As used herein, “a non-specific DNase” refers to an enzyme that cleaves a DNA molecule irrespective of the sequence of nucleotides present at the cleavage site. An exemplary non-specific DNase is DNase I.

As used herein, the term “single-base cutter” refers to a restriction enzyme that recognizes and cleaves a particular base (e.g., A, C, T or G for DNA or A, C, U or G for RNA), or a particular type of base (e.g., purines or pyrimidines).

As used herein, the term “1¼-cutter” refers to a restriction enzyme that recognizes and cleaves a 2 base stretch in the nucleic acid, in which the identity of one base position is fixed and the identity of the other base position is any three of the four typically occurring bases.

As used herein, the term “1½-cutter” refers to a restriction enzyme that recognizes and cleaves a 2 base stretch in the nucleic acid, in which the identity of one base position is fixed and the identity of the other base position is any two out of the four typically occurring bases.

As used herein, the term “double-base cutter” or “2 cutter” refers to a restriction enzyme that recognizes and cleaves a specific nucleic acid site that is 2 bases long.

As used herein, the phrase “set of mass signals” refers to two or more mass determinations made for two or more nucleic acid fragments.

As used herein, scoring or a score refers to a calculation of the probability that a particular sequence variation candidate is actually present in the target nucleic acid or protein sequence. The value of a score is used to determine the sequence variation candidate that corresponds to the actual target sequence. Usually, in a set of samples of target sequences, the highest score represents the most likely sequence variation in the target molecule, but other rules for selection also can be used, such as detecting a positive score, when a single target sequence is present.

As used herein, simulation (or simulating) refers to the calculation of a fragmentation pattern based on the sequence of a nucleic acid or protein and the predicted cleavage sites in the nucleic acid or protein sequence for a particular specific cleavage reagent. The fragmentation pattern can be simulated as a table of numbers (for example, as a list of peaks corresponding to the mass signals of fragments of a reference biomolecule), as a mass spectrum, as a pattern of bands on a gel, or as a representation of any technique that measures mass distribution. Simulations can be performed in most instances by a computer program.

As used herein, simulating cleavage refers to an in silico process in which a target molecule or a reference molecule is virtually cleaved.

As used herein, in silico refers to research and experiments performed using a computer. In silico methods include, but are not limited to, molecular modelling studies, biomolecular docking experiments, and virtual representations of molecular structures and/or processes, such as molecular interactions.

As used herein, the phrase “constructing a nucleotide sequence” refers to the process of elucidating the nucleotide sequence of the target nucleic acid molecule using any one of a variety of algorithms that can be designed for such construction.

As used herein, a subject includes, but is not limited to, animals, plants, bacteria, viruses, parasites and any other organism or entity that has nucleic acid. Among subjects are mammals, preferably, although not necessarily, humans. A patient refers to a subject afflicted with a disease or disorder.

As used herein, a phenotype refers to a set of parameters that includes any distinguishable trait of an organism. A phenotype can be physical traits and can be, in instances in which the subject is an animal, a mental trait, such as emotional traits.

As used herein, ?assignment? refers to a determination that the position of a nucleic acid or protein fragment indicates a particular molecular weight and a particular terminal nucleotide or amino acid.

As used herein, “a” refers to one or more.

As used herein, “plurality” refers to two or more. For example, a plurality of polynucleotides or polypeptide refers to two or more polynucleotides or polypeptides, each of which has a different sequence. Such a difference can be due to a naturally occurring variation among the sequences, for example, to an allelic variation in a nucleotide or an encoded amino acid, or can be due to the introduction of particular modifications into various sequences, for example, the differential incorporation of mass modified nucleotides into each nucleic acid or protein in a plurality.

As used herein, “unambiguous” refers to the unique assignment of peaks or signals corresponding to a particular sequence variation, such as a mutation, in a target molecule and, in the event that a number of molecules or mutations are multiplexed, that the peaks representing a particular sequence variation can be uniquely assigned to each mutation or each molecule.

As used herein, a data processing routine refers to a process, that can be embodied in software, that determines the biological significance of acquired data (i.e., the ultimate results of the assay). For example, the data processing routine can make a genotype determination based upon the data collected. In the systems and methods herein, the data processing routine also can control the instrument and/or the data collection routine based upon the results determined. The data processing routine and the data collection routines can be integrated and provide feedback to operate the data acquisition by the instrument, and hence provide the assay-based judging methods provided herein.

As used herein, a plurality of genes includes at least two, five, 10, 25, 50, 100, 250, 500, 1000, 2,500, 5,000, 10,000, 100,000, 1,000,000 or more genes. A plurality of genes can include complete or partial genomes of an organism or even a plurality thereof. Selecting the organism type determines the genome from among which the gene regulatory regions are selected. Exemplary organisms for gene screening include animals, such as mammals, including human and rodent, such as mouse, insects, yeast, bacteria, parasites, and plants.

As used herein, “sample” refers to a composition containing a material to be detected. In a preferred embodiment, the sample is a “biological sample.” The term “biological sample” refers to any material obtained from a living source, for example, an animal such as a human or other mammal, a plant, a bacterium, a fungus, a protist or a virus. The biological sample can be in any form, including a solid material such as a tissue, cells, a cell pellet, a cell extract, or a biopsy, or a biological fluid such as urine, blood, plasma, serum, saliva, sputum, amniotic fluid, exudate from a region of infection or inflammation, or a mouth wash containing buccal cells, cerebral spinal fluid, synovial fluid, organs, semen, ocular fluid, mucus, secreted fluids such as gastric fluids or breast milk, and pathological samples such as a formalin-fixed sample embedded in paraffin. Preferably solid materials are mixed with a fluid. In particular, herein, the sample can be mixed with matrix when mass spectrometric analyses of biological material such as nucleic acids is performed. Derived from means that the sample can be processed, such as by purification or isolation and/or amplification of nucleic acid molecules.

As used herein, a composition refers to any mixture. It can be a solution, a suspension, liquid, powder, a paste, aqueous, non-aqueous or any combination thereof.

As used herein, a combination refers to any association between two or among more items.

As used herein, the term “amplicon” refers to a region of DNA that can be replicated.

As used herein, the term “complete cleavage” or “total cleavage” refers to a cleavage reaction in which all the cleavage sites recognized by a particular cleavage reagent are cut to completion.

As used herein, the term “false positives” refers to signals that are above background noise and not generated as a result of an expected event. For example, a false positive can arise when a mass peak that does not reflect the target nucleic acid nucleotide sequence is observed, or when a fragment is formed by a process other than specific actual or simulated cleavage of a nucleic acid or protein.

As used herein, the term “false negatives” refers to actual signals that are missing from an actual measurement, but were otherwise expected. For example, a false negative can arise when mass signals not observed in an actual mass spectrum were calculated to be present in a corresponding simulated spectrum.

As used herein, fragment or cleave means any manner in which a nucleic acid or protein molecule is separated into smaller pieces. Fragmentation or cleavage methods include physical cleavage, enzymatic cleavage, chemical cleavage and any other way smaller pieces of a nucleic acid are produced.

As used herein, fragmentation conditions or cleavage conditions refers to the set of one or more fragmentation reagents, buffers, or other chemical or physical conditions that can be used to perform actual or simulated cleavage reactions. Such conditions include parameters of the reactions such as, time, temperature, pH, or choice of buffer.

As used herein, uncleaved cleavage sites means cleavage sites that are known recognition sites for a cleavage reagent but that are not cut by the cleavage reagent under the conditions of the reaction, e.g., time, temperature, or modifications of the bases at the cleavage recognition sites to prevent cleavage by the reagent.

As used herein, complementary cleavage reactions refers to cleavage reactions that are carried out or simulated on the same target or reference nucleic acid or protein using different cleavage reagents or by altering the cleavage specificity of the same cleavage reagent such that alternate cleavage patterns of the same target or reference nucleic acid or protein are generated.

As used herein, fluid refers to any composition that can flow. Fluids thus encompass compositions that are in the form of semi-solids, pastes, solutions, aqueous mixtures, gels, lotions, creams and other such compositions.

As used herein, a cellular extract refers to a preparation or fraction which is made from a lysed or disrupted cell.

As used herein, a kit is combination in which components are packaged optionally with instructions for use and/or reagents and apparatus for use with the combination.

As used herein, a system refers to the combination of elements with software and any other elements for controlling and directing methods provided herein.

As used herein, software refers to computer readable program instructions that, when executed by a computer, performs computer operations. Typically, software is provided on a program product containing program instructions recorded on a computer readable medium, such as but not limited to, magnetic media including floppy disks, hard disks, and magnetic tape; and optical media including CD-ROM discs, DVD discs, magneto-optical discs, and other such media on which the program instructions can be recorded.

As used herein, the phrase target nucleic acid or target nucleic acid molecule refers to the nucleic acid molecule that is of interest to be analyzed. The target nucleic acid molecule can be either a single-stranded or double-stranded molecule.

As used herein, the phrase “partially digested” means that only a subset of the restriction sites are cleaved.

As used herein, “controlling the complexity” and grammatical variants thereof, refers to methods for manipulating the number, variability, or number and variability of nucleic acid molecules having different nucleotide sequences. For example controlling the complexity of target nucleic acid fragments hybridized to a capture oligonucleotide refers to manipulating experimental conditions to control the number, variability, or number and variability of target nucleic acid fragments having different nucleotide sequences, that hybridize to a particular capture oligonucleotide probe sequence. The number of different target nucleic acid sequences that hybridize to a capture oligonucleotide probe refers to the quantity of non-identical target nucleic acids or target nucleic acid fragments that hybridize to at least a portion of a particular nucleotide sequence of a capture oligonucleotide probe. For example, two or more target nucleic acid fragments that have sequences different from each other can hybridize to a single array position where all of the capture oligonucleotide probes of that single array position have the same nucleotide sequence. In one example, two target nucleic acids that have different sequences can hybridize to a capture oligonucleotide where the hybridization entails base-pairing between the capture oligonucleotide and two different nucleotide sequences of the target nucleic acid fragments. Thus, in one embodiment of the methods disclosed herein, the capture oligonucleotides are capable of base-pairing with two or more different nucleotide sequences. The variability of different target nucleic acid sequences that hybridize to a capture oligonucleotide probe refers to the degree of sequence identity, both in terms of length and nucleotide sequence, of the different target nucleic acid sequences that hybridize to a capture oligonucleotide probe.

As used herein, “modulating” the number of sequences that hybridize to a capture oligonucleotide probe refers to setting or modifying conditions in order to set or modify the number, variability, or number and variability of the sequences of target nucleic acid fragments that hybridize to a capture oligonucleotide probe. Exemplary conditions that can be set or modified are provided hereinabove. Accordingly, the complexity of the target nucleic acid fragments hybridized to a capture oligonucleotide probe can be controlled by modulating the number of target nucleic acid sequences that hybridize to a capture oligonucleotide probe, which can be accomplished by setting or modifying the conditions that affect the number, variability, or number and variability of target nucleic acid fragments that hybridize to a capture oligonucleotide probe.

As used herein the phrase “semi-specific capture” refers to the binding of 2 or more different target nucleic acid fragments to a single capture oligonucleotide sequence, that can be partially degenerate or may not contain any degenerate nucleotide bases. Semi-specific capture does not include binding all target nucleic acid fragments or randomly binding nucleic acid fragments, but instead refers to binding 2 or more target nucleic acid fragments in preference over at least one other target nucleic acid fragment.

Use of the term “unique” and the phrase “identical sequence” in describing the nucleotide sequences of capture oligonucleotides of an array refers to strict identity; thus, where a first oligonucleotide has the sequence ATCG and a second oligonucleotide has a sequence ATCGA, the two oligonucleotides are unique, and do not have the identical sequence. Similarly, as used herein, reference to one or more of target nucleic acids or target nucleic acid fragments that hybridize to a capture oligonucleotide, unless otherwise noted, refers to each of one or more target nucleic acids or target nucleic acid fragments binding separately to one of a plurality of capture oligonucleotide probes that have identical sequences. Typically, one or more target nucleic acids or target nucleic acid fragments hybridize to a capture oligonucleotide at a particular array position.

As used herein, the phrase “partially degenerate capture oligonucleotides” refers to oligonucleotides that hybridize to at least two different nucleotide sequences with similar specificity, but do not bind all possible nucleotide sequences with similar specificity. For example, a partially degenerate capture oligonucleotide can be an oligonucleotide containing a universal base.

As used herein, the phrase “all theoretical combinations” refers to the complete group of oligonucleotides of a given length, such that all possible nucleotide sequences of that length are represented.

As used herein, “degenerate base” refers to either a “universal base” or a “semi-universal base” or other base that can base pair with similar specificity to two or more bases of a target nucleic acid or target nucleic acid fragment.

As used herein a “universal base” refers to a base that can bind to any of the 4 nucleotides present in genomic DNA, without any substantial discrimination. Exemplary universal bases for use herein include Inosine, Xanthosine, 3-nitropyrrole (Bergstrom et al., Abstr. Pap. Am. Chem. Soc. 206(2):308 (1993); Nichols et al., Nature 369:492-493; Bergstrom et al., J. Am. Chem. Soc. 117:1201-1209 (1995)), 4-nitroindole (Loakes et al., Nucleic Acids Res., 22:4039-4043 (1994)), 5-nitroindole (Loakes et al. (1994)), 6-nitroindole (Loakes et al. (1994)); nitroimidazole (Bergstrom et al., Nucleic Acids Res. 25:1935-1942 (1997)), 4-nitropyrazole (Bergstrom et al. (1997)), 5-aminoindole (Smith et al., Nucl. Nucl. 17:555-564 (1998)), 4-nitrobenzimidazole (Seela et al., Helv. Chim. Acta 79:488-498 (1996)), 4-aminobenzimidazole (Seela et al., Helv. Chim. Acta 78:833-846 (1995)), phenyl C-ribonucleoside (Millican et al., Nucleic Acids Res. 12:7435-7453 (1984); Matulic-Adamic et al., J. Org. Chem. 61:3909-3911 (1996)), benzimidazole (Loakes et al., Nucl. Nucl. 18:2685-2695 (1999); Papageorgiou et al., Helv. Chim. Acta 70:138-141 (1987)), 5-fluoroindole (Loakes et al. (1999)), indole (Girgis et al., J. Heterocycle Chem. 25:361-366 (1988)); acyclic sugar analogs (Van Aerschot et al., Nucl. Nucl. 14:1053-1056 (1995); Van Aerschot et al., Nucleic Acids Res. 23:4363-4370 (1995); Loakes et al., Nucl. Nucl. 15:1891-1904 (1996)), including derivatives of hypoxanthine, imidazole 4,5-dicarboxamide, 3-nitroimidazole, 5-nitroindazole; aromatic analogs (Guckian et al., J. Am. Chem. Soc. 118:8182-8183 (1996); Guckian et al., J. Am. Chem. Soc. 122:2213-2222 (2000)), including benzene, naphthalene, phenanthrene, pyrene, pyrrole, difluorotoluene; isocarbostyril nucleoside derivatives (Berger et al., Nucleic Acids Res. 28:2911-2914 (2000); Berger et al., Angew. Chem. Int. Ed. Engl., 39:2940-2942 (2000)), including MICS, ICS; hydrogen-bonding analogs, including N8-pyrrolopyridine (Seela et al., Nucleic Acids Res. 28:3224-3232 (2000)); and LNAs such as aryl-β-C-LNA (Babu et al., Nucleosides, Nucleotides & Nucleic Acids 22:1317-1319 (2003); WO 03/020739).

As used herein, the phrase “semi-universal base” refers to a base that preferentially binds to 2 or 3 of the deoxyribonucleotides, but does not bind to all 4 typically-occurring nucleotides (i.e., A, C, G and T in DNA and A, C, G and U in RNA) with the same or similar specificity. For example, a semi-universal base binds to 2 or 3 typically-occurring nucleotides at a much greater level than it binds to at least one other typically-occurring nucleotide.

As used herein, a “solid support” (also referred to as an insoluble support or solid support) refers to any solid or semisolid or insoluble support to which a molecule of interest, typically a biological molecule, organic molecule or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications.

As used herein, a “portion” of a nucleic acid such as a target nucleic acid or a reference nucleic acid, refers to a nucleotide sequence or a region of a nucleic acid that does not encompass the entire nucleic acid. For example, a portion can be a short nucleotide sequence, such as a SNP, methylated C, or microsatellite of a nucleic acid. A portion also can be, for example, a particular fragment of a nucleic acid of known or unknown nucleotide sequence, where the fragment can arise, for example, as a result of a difference in sequence due to variation between organisms, strains or species, and where the fragment is formed using the methods disclosed herein. A portion also can be a region of a nucleic acid that differently interacts, or is differently treated, relative to another region.

B. Methods for Sequencing Nucleic Acid Molecules

Provided herein are methods for sequencing nucleic acids, by
a) generating overlapping fragments of a target nucleic acid;
b) hybridizing the fragments to an array of capture oligonucleotides on a solid support under conditions that do not eliminate mismatched hybridization to form an array of captured fragments;
c) determining the mass of the captured fragments at each array position using mass spectrometric analysis; and
d) constructing a nucleotide sequence of the target nucleic acid from a set of mass signals acquired from each array position.
   Also provided herein are methods for sequencing nucleic acids, comprising
a) generating overlapping fragments of a target nucleic acid;
b) hybridizing the fragments to an array of capture oligonucleotides on a solid support to form an array of captured fragments, wherein an at least a subset of the capture oligonucleotides are partially degenerate;
c) determining the mass of the captured fragments at each array position using mass spectrometric analysis; and
d) constructing a nucleotide sequence of the target nucleic acid from a set of mass signals acquired from each array position.
   Also provided herein are methods for sequencing nucleic acids, comprising
a) generating overlapping fragments of a target nucleic acid;
b) hybridizing the fragments to an array of capture oligonucleotides on a solid support to form an array of captured fragments, wherein an at least one capture oligonucleotide hybridizes to two or more fragments;
c) determining the mass of the captured fragments at each array position using mass spectrometric analysis; and
d) constructing a nucleotide sequence of the target nucleic acid from a set of mass signals acquired from each array position.
   In certain embodiments of each of these methods provided herein, the overlapping fragments of a target-nucleic acid are generated randomly.

In another embodiment for each of these methods provided herein, prior to step c) of determining the mass of the captured fragments, the hybridized fragments are re-solubilized in a solution. Such re-solubilization permits the well-known use of, for example, a pin array that is dipped into the solution containing the re-solubilized fragments to transfer the fragments to an appropriate chip for mass spectrometry analysis.

As set forth above, the methods provided herein permit a longer target nucleic acid sequence read length than can be achieved using SBH and/or mass spectrometric analysis of target nucleic acid bound to a solid-phase chip. In another embodiment, a multiplicity of target nucleic acid fragments of shorter lengths, (such as, e.g., 200, 300, 400, 500, 600, 700, 800, 900, 1,000, 1,500 bases) can be sequenced or analyzed by the methods provided herein. The methods herein include analysis of 5, 10, 15, 20, 50, 100, 200, 500 or more nucleic acid fragments. These multiple shorter sequence sets are useful, for example, in re-sequencing methods when part of a particular sequence is known. These multiple shorter sequence sets also are useful for multiplexed genotyping, haplotyping, SNP and methylation detection methods.

C. Target Nucleic Acid Molecules

The target nucleic acid molecule can be either a single-stranded or double-stranded nucleic acid molecule. In particular embodiments, RNA is used rather than DNA when using MALDI-TOF MS analysis, or when an RNA transcription based approach would increase the yield of fragments hybridized onto the chip or when RNA hybridized to DNA capture oligos would permit further modifications after hybridization. In another embodiment, DNA is used and is hybridized to DNA capture oligos; further modifications after hybridization also can be accomplished for the DNA:DNA hybrids.

1. Sources

The target nucleic acids can be selected from among single-stranded DNA, double-stranded DNA, cDNA, single-stranded RNA, double-stranded RNA, DNA/RNA hybrid and a DNA/RNA mosaic nucleic acid. The target nucleic acids also can include modified nucleic acids such as methylated DNA and RNA containing, for example, pseudouridine. The target nucleic acids can be directly isolated from a biological sample, or can be derived by amplification or cloning of nucleic acid fragments from a biological sample. Target nucleic acids that serve as the template for cloning or amplification can be whole, in-tact target nucleic acids, or target nucleic acid fragments, where the target nucleic acid fragments can be of the length desired for hybridization or mass measurement, or can be of intermediary length where the target nucleic acid fragments are first amplified and then subjected to one or more additional fragmentation steps.

The samples used in the methods described herein can be selected according to the purpose of the method to be applied. For example, a sample can be from a single individual, where the sample is examined to determine the nucleotide sequence at one or more loci for the individual. One skilled in the art can use the methods described herein to determine the desired sample to be examined.

A sample can be from any subject, including animal, plant, bacterium, virus, parasite, bird, reptile, amphibian, fungus, fish, and other plants and animals. Among subjects are mammals, typically humans. A sample from a subject can be in any form, including a solid material such as a tissue, cells, a cell pellet, a cell extract, or a biopsy, or a biological fluid such as urine, blood, interstitial fluid, peritoneal fluid, plasma, lymph, ascites, sweat, saliva, follicular fluid, breast milk, non-milk breast secretions, serum, cerebral spinal fluid, feces, seminal fluid, lung sputum, amniotic fluid, exudate from a region of infection or inflammation, a mouth wash containing buccal cells, synovial fluid, or any other fluid sample produced by the subject. In addition, the sample can be collected tissues, including bone marrow, epithelium, stomach, prostate, kidney, bladder, breast, colon, lung, pancreas, endometrium, neuron, and muscle. Samples can include tissues, organs, and pathological samples such as a formalin-fixed sample embedded in paraffin.

2. Preparation

As one of skill in the art recognize, some samples can be used directly in the methods provided herein. For example, samples can be examined using the methods described herein without any purification or manipulation steps to increase the purity of desired cells or nucleic acid molecules.

If desired, a sample can be prepared using known techniques, such as that described by Maniatis, et al. (Molecular Cloning: A Laboratory Manual, Cold Spring Harbor, N.Y., pp. 280-281 (1982)). For example, samples examined using the methods described herein can be treated in one or more purification steps in order to increase the purity of the desired cells or nucleic acid in the sample. If desired, solid materials can be mixed with a fluid.

Methods for isolating nucleic acid in a sample from essentially any organism or tissue or organ in the body, as well as from cultured cells, are well known. For example, the sample can be treated to homogenize an organ, tissue or cell sample, and the cells can be lysed using known lysis buffers, sonication, electroporation and methods and combinations thereof. Further purification can be performed as needed, as is appreciated by those skilled in the art. In addition, sample preparation can include a variety of reagents which can be included in subsequent steps. These include reagents such as salts, buffers, neutral proteins (e.g., albumin), detergents, and such reagents, which can be used to facilitate optimal hybridization or enzymatic reactions, and/or reduce non-specific or background interactions. Also, reagents that otherwise improve the efficiency of the assay, such as, for example, protease inhibitors, nuclease inhibitors and anti-microbial agents, can be used, depending on the sample preparation methods and purity of the target nucleic acid molecule.

3. Size and Composition of Target Nucleic Acid Molecule

The length of the target nucleic acid molecule that can be used can vary according to the sequence of the target nucleic acid molecule, the particular methods used for fragmentation, the particular methods can capture oligonucleotides used for hybridization, the percentage of the total target nucleic acid molecule for which the nucleotide sequence is to be determined, the desired level of accuracy in sequence determination, and the nature of the sequencing (e.g., de novo sequencing verus resequencing). For example, the length of the target nucleic acid molecule can be limited to a length in which the nucleotide sequence of at least about 1%, at least about 3%, at least about 5%, at least about 10%, at least about 20%, at least about 30%, at least about 40%, at least about 50%, at least about 60%, at least about 70%, at least about 80%, at least about 85%, at least about 90%, at least about 95%, at least about 98%, at least about 99%, or all of the target nucleic acid molecule can be determined using the fragmentation and detection methods disclosed herein. For example, a target nucleic acid molecule can be at least about 20, 25, 30, 35, 40, 50, 60, 70, 80, 90, 100, 120, 140, 160, 180, 200, 225, 250, 275, 300, 350, 400, 450, 500, 550, 600, 700, 800, 900, 1000, 1200, 1400, 1600, 1800, 2000, 2500 or 3000 bases in length. Typically, a target nucleic acid molecule is no longer than about 10,000, 5000, 4000, 3000, 2500, 2000, 1500, 1000, 900, 800, 700, 600, 500, 450, 400, 350, 280, 260, 240, 220, 200, 190, 180, 170, 160, 150, 140, 130, 120, 110 or 100 bases in length.

4. Amplification

In some embodiments, target nucleic acid molecules can be amplified to increase the number of nucleic acid molecules that can be treated and measured in subsequent steps, and, optionally, to treat the target nucleic acid sequence. Amplification can be achieved by polymerase chain reaction (PCR), reverse transcription followed by the polymerase chain reaction (RT-PCR), rolling circle amplification, whole genome amplification, strand displacement amplification (SDA), and by transcription based processes. Amplification methods can have varied the reaction conditions and/or the reactants in a variety of different amplification methods that can create a variety of different amplification products.

a. Reaction Parameters

Amplification steps can be performed in which complementary strands, if present, are separated, primers are hybridized to the strands, and the primers have added thereto nucleotides to form a new complementary strand. Strand separation can be effected either as a separate step or simultaneously with the synthesis of the primer extension products. This strand separation can be accomplished using various suitable denaturing conditions, including physical, chemical, or enzymatic means, the word “denaturing” includes all such means. One physical method of separating nucleic acid strands involves heating the target nucleic acid molecule until it is denatured. Typical heat denaturation can involve temperatures ranging from about 80 EC to 105 EC, for times ranging from about 1 to 10 minutes. Strand separation also can be accomplished by chemical means, including high salt conditions or strongly basic conditions. Strand separation also can be induced by an enzyme from the class of enzymes known as helicases or by the enzyme RecA, which has helicase activity, and in the presence of riboATP, is known to denature DNA. The reaction conditions suitable for strand separation of nucleic acids with helicases are described by Kuhn Hoffmann-Berling, CSH-Quantitative Biology, 43:63 (1978) and techniques for using RecA are reviewed in C. Radding, Ann. Rev. Genetics 16:405-437 (1982).

After each amplification step, the amplified product typically is double stranded, with each strand complementary to the other. The complementary strands can be separated, and both separated strands can be used as a template for the synthesis of additional nucleic acid strands. This synthesis can be performed under conditions allowing hybridization of primers to templates to occur. Generally synthesis occurs in a buffered aqueous solution, typically at about a pH of 7-9, such as about pH 8. Typically, a molar excess of two oligonucleotide primers can be added to the buffer containing the separated template strands. In some embodiments, the amount of target nucleic acid is not known (for example, when the methods disclosed herein are used for diagnostic applications), so that the amount of primer relative to the amount of complementary strand cannot be determined with certainty.

In an exemplary method, deoxyribonucleoside triphosphates dATP, dCTP, dGTP, and dTTP can be added to the synthesis mixture, either separately or together with the primers, and the resulting solution can be heated to about 90 EC-100 EC from about 1 to 10 minutes, typically from 1 to 4 minutes. After this heating period, the solution can be allowed to cool to about room temperature. To the cooled mixture can be added an appropriate enzyme for effecting the primer extension reaction (called herein “enzyme for polymerization”), and the reaction can be allowed to occur under conditions known in the art. This synthesis (or amplification) reaction can occur at room temperature up to a temperature above which the enzyme for polymerization no longer functions. For example, the enzyme for polymerization also can be used at temperatures greater than room temperature if the enzyme is heat stable. In one embodiment, the method of amplifying is by PCR, as described herein and as is commonly used by those of skill in the art. Alternative methods of amplification have been described and also can be employed. A variety of suitable enzymes for this purpose are known in the art and include, for example, E. coli DNA polymerase I, Klenow fragment of E. coli DNA polymerase I, T4 DNA polymerase, other available DNA polymerases, polymerase muteins, reverse transcriptase, and other enzymes, including thermostable enzymes (i.e., those enzymes which perform primer extension at elevated temperatures, typically temperatures that cause denaturation of the nucleic acid to be amplified).

b. Modified Nucleosides

In one embodiment, the target nucleic acids are amplified using modified nucleosides, such as modified nucleoside triphosphates. Some modifications can confer or alter cleavage specificity of the target nucleic acid sequence by the respective cleavage methods. Other modifications, such as mass modifications, can alter the mass of the target nucleic acid amplified nucleic acids and fragments thereof. Other nucleosides can alter the functional properties of a polynucleotide, including, but not limited to increasing the sensitivity of a polynucleotide to fragmentation, decreasing the ability to further extend the polynucleotide. Modified nucleosides are not necessarily non-naturally occurring, but are simply nucleosides that are not typically incorporated into a particular polynucleotide (e.g., nucleosides other than A, C, T and G when DNA is formed, or nucleosides other than A, C, U and G when RNA is formed).

In one embodiment, the target nucleic acids are amplified using nucleoside triphosphates that are naturally occurring, but that are not normal precursors of the target nucleic acid. For example, one rNTP and three dNTPs can be incorporated into the amplified polynucleotide (e.g., rCTP, dATP, dTTP and dGTP). In another example, deoxyuridine triphosphate, which is not normally present in DNA, can be incorporated into an amplified DNA molecule by amplifying the DNA in the presence of normal DNA precursor nucleotides (e.g. dCTP, dATP, and dGTP) and dUTP. Such an incorporation of uridine into DNA can facilitate base-specific cleavage of DNA. For example, when amplified uridine-containing DNA is treated with uracil-DNA glycosylase (UDG), uracil residues are cleaved. Subsequent chemical treatment of the products from the UDG reaction results in the cleavage of the phosphate backbone and the generation of nucleobase specific fragments. Moreover, the separation of the complementary strands of the amplified product prior to glycosylase treatment allows complementary patterns of fragmentation to be generated. Thus, the use of dUTP and Uracil DNA glycosylase allows the generation of T specific fragments for the complementary strands, providing information on the T as well as the A positions within a given sequence.

Amplification, or other nucleotide synthetic reactions such as transcription, can be carried out using a nucleotide analog that can serve to terminate elongation, such as a didexoynucleotide. In one embodiment, the reaction conditions contain one of the four nucleotide monomers typically incorporated into the oligonucleotide in dideoxynucleotide form. In other embodiments, the reaction conditions contain two of the four, three of the four, or all four of the nucleotide monomers in dideoxynucleotide form. The reaction conditions can contain any possible mixture of a particular nucleotide monomer in ribonucleotide, deoxynucleotide and/or in dideoxyribonucleotide form. For example, adenosine (A) can be present in a reaction mixture as 10% ribonucleotide, 80% deoxynucleotide and 10% dideoxynucleotide form. Amplification or other reactions such as transcription need not be carried out to completion. For example, an amplification step in PCR can be quenched before all primers are fully extended, resulting in target fragment nucleic acids of a variety of different lengths. Thus, in one embodiment, a reaction can be carried out in such a manner as to yield a heterogenous pool of target nucleic acids, representing oligonucleotides terminated at different locations during elongation.

In one embodiment, one or more of the nucleoside triphosphates can be substituted with an analog that creates a selectively non-hydrolyzable bond between nucleotides. For example, a nucleoside can be substituted with an α-thio-substrate and the phosphorothioate internucleoside linkages can subsequently be modified by alkylation using reagents such as an alkyl halide (e.g., iodoacetamide, iodoethanol) or 2,3-epoxy-1-propanol. Other exemplary nucleosides that can be selectively non-hydrolyzable include 2′fluoro nucleosides, 2′deoxy nucleosides and 2′amino nucleosides.

Mass modified nucleosides can be selected from among mass modified deoxynucleoside triphosphates, mass modified dideoxynucleoside triphosphates, and mass modified ribonucleoside triphosphates. Mass modified nucleoside triphosphates can be modified on the base, the sugar, and/or the phosphate moiety, and are introduced through an enzymatic step, chemically, or a combination of both. In one aspect, the modification can include 2′ substituents other than a hydroxyl group. In another aspect, the internucleoside linkages can be modified e.g., phosphorothioate linkages or phosphorothioate linkages further reacted with an alkylating agent.

In yet another aspect, the modified nucleoside triphosphate can be modified with a methyl group, e.g., 5-methyl cytosine or 5-methyl uridine. Other known mass-modifying moieties include substitutions of H for halogens like F, Cl, Br and/or I, or pseudohalogens such as SCN, NCS, or by using different alkyl, aryl or aralkyl moieties such as methyl, ethyl, propyl, isopropyl, t-butyl, hexyl, phenyl, substituted phenyl, benzyl, or functional groups such as CH₂F, CHF₂, CF₃, Si(CH₃)₃, Si(CH₃)₂(C₂H₅), Si(CH₃)(C₂H₅)₂, Si(C₂H₅)₃. Yet another mass-modification can be obtained by attaching homo- or heteropeptides through the nucleic acid molecule (e.g., detector (D)) or nucleoside triphosphates.

One example useful in generating mass-modified species with a mass increment of 57 is the attachment of oligoglycines, e.g., mass-modifications of 74 (r=1, m=0), 131 (r=1, m=2), 188 (r=1, m=3), 245 (r=1, m=4) are achieved. Simple oligoamides also can be used, e.g., mass-modifications of 74 (r=1, m=0), 88 (r=2, m=0), 102 (r=3, m=0), 116 (r=4, m=0), etc. are obtainable.

Mass modifying moieties can be attached, for instance, to either the 5′-end of the oligonucleotide, to the nucleobase (or bases), to the phosphate backbone, to the 2′-position of the nucleoside (nucleosides), and/or to the terminal 3′-position. Examples of mass modifying moieties include, for example, a halogen, an azido, or of the type, XR, wherein X is a linking group and R is a mass-modifying functionality. A mass-modifying functionality can, for example, be used to introduce defined mass increments into the oligonucleotide molecule, as described herein. Modifications introduced at the phosphodiester bond such as with alpha-thio nucleoside triphosphates, have the advantage that these modifications do not interfere with accurate Watson-Crick base-pairing and additionally allow for the one-step post-synthetic site-specific modification of the complete nucleic acid molecule e.g., via alkylation reactions (see, e.g., Nakamaye et al., Nucl. Acids Res. 16:9947-9959 (1988)). Exemplary mass-modifying functionalities are boron-modified nucleic acids, which can be efficiently incorporated into nucleic acids by polymerases (see, e.g., Porter et al. Biochemistry 34:11963-11969 (1995); Hasan et al., Nucl. Acids Res. 24:2150-2157 (1996); Li et al. Nucl. Acids Res. 23:4495-4501 (1995)).

Furthermore, the mass-modifying functionality can be added so as to affect chain termination, such as by attaching it to the 3′-position of the sugar ring in the nucleoside triphosphate. For those skilled in the art, it is clear that many combinations can be used in the methods provided herein. In the same way, those skilled in the art recognize that chain-elongating nucleoside triphosphates also can be mass-modified in a similar fashion with numerous variations and combinations in functionality and attachment positions.

Different mass-modified nucleotides can be used to simultaneously detect a variety of different nucleic acid fragments simultaneously. In one embodiment, mass modifications can be incorporated during the amplification process. In another embodiment, multiplexing of different target nucleic acid molecules can be performed by mass modifying one or more target nucleic acid molecules, where each different target nucleic acid molecule can be differently mass modified, if desired.

c. Amplification Methods

Amplification methods can be used to create a variety of different amplification products, according to the desired assay design.

In one embodiment, provided herein are nucleotide products of amplification or other reactions such as transcription, where the product nucleotides can differ in size, even when a single template size is provided. For example, product nucleotides can be overlapping, such that one or more nucleotide positions from the native target nucleic acid are in common between two or more product nucleotides. Such overlapping nucleotides include “ladder” nucleotides in which a series of nucleotides of different sizes share the same core sequence and consecutively larger nucleotides contain additional nucleotides, typically at only the 3′ or 5′ end of the nucleotide, in increments of one or more nucleic acid positions. A variety of methods can be used to form such products, including, but not limited to nucleic acid synthesis reaction with one of the four nucleosides being present in a combination of both dideoxy and non-dideoxy nucleosides.

In other embodiments, amplification or other nucleotide synthetic reactions can be carried out using one or more primers that hybridize to both a constant region and a variable region in a template target nucleic acid or template target nucleic acid fragment. For example, a target nucleic acid molecule can be fragmented using the methods disclosed herein; such target nucleic acid fragments can have ligated thereto, one or more adaptor oligonucleotides whereby adaptor oligonucleotides having the same sequence are ligated to the same end (i.e., 3′ end or 5′ end) of two or more target nucleic acid fragments having different sequences. Each ligation product contains both a target nucleic acid fragment and the adaptor oligonucleotide. The primers can hybridize to some, but not all ligation products by hybridizing to at least a portion of the adaptor oligonucleotide region and to at least a portion of some, but not all target nucleic acid fragments, since the portion of the target nucleic acid fragments varies from fragment to fragment. Amplification or other nucleotide synthetic reactions are then only carried out for the subset of target nucleic acid fragments that hybridize with the primers in the variable region of the ligated fragment. In this way, a set of one or more primers can be used to amplify a subpopulation of all target nucleic acid fragments, according to which variable sequences of target nucleic acid fragments hybridize with primers. In one embodiment, only one primer sequence is used to ligate to either the 3′ end, 5′ end, or both the 3′ end and 5′ end of target nucleic acid fragments. In another embodiment, two primers are used to ligate to target nucleic acid fragments: a first is ligated to the 3′ target nucleic acid fragment end, and a second is ligated to the 5′ target nucleic acid fragment end. In another embodiment, two or more primers are used to ligate to either the 3′ or 5′ end. For example, a plurality of primers that recognize different constant regions can be used such that a first set of primers hybridizes to a first population of target nucleic acid fragments and a second set of primers hybridizes to a second population of target nucleic acid fragments; typically, the first and second populations of target nucleic acids have no overlapping members.

Selective nucleotide synthesis also can be performed in conjunction with fragmentation. A target nucleic acid amplified through a plurality of nucleic acid synthesis cycles use primers hybridizing to two separate regions of the target nucleic acid molecule. Fragmentation of a target nucleic acid molecule in the center region in between the two primer hybridization sites prevent amplification of the target nucleic acid molecule. Hence selective fragmentation of the center region of nucleic acid molecules can result in selective amplification of a target nucleic acid molecule even if the primers used in the nucleic acid synthesis reactions are not selective or are not highly selective.

In one example, the sample can be treated with fragmentation conditions prior to being treated with nucleic acid synthesis conditions. In such an example, the fragmentation conditions can selectively cleave particular nucleotide sequences. For example, a sample can have added thereto a restriction endonuclease, such as EcoRI. This results in a sample containing cleaved target nucleic acid molecules that contained the EcoRI recognition site, and intact target nucleic acid molecules that do not contain the EcoRI recognition site. The sample then can be treated with nucleic acid synthesis conditions using primers designed so that only uncleaved target nucleic acid molecules are amplified. As a result of the cleavage, amplification is selective for a subset of all target nucleic acid molecules according to the presence of a restriction endonuclease recognition site. Fragmentation conditions that can be used in the methods provided herein include any fragmentation conditions that can selectively cleave nucleic acid molecules, including restriction endonucleases. Additional fragmentation conditions that can be used include any fragmentation condition that can cleave by sequence specificity.

In another embodiment, transcription can be performed as the only nucleic acid amplification method, or in addition to other nucleic acid amplification methods. Transcription methods, which use a template DNA molecule to form an RNA molecule, can serve to amplify target nucleic acid molecules and to modify target nucleic acid molecule from a DNA form to a RNA form. Exemplary template DNA includes an amplified product target nucleic acid molecule and treated, unamplified target nucleic acid molecule.

As described herein, a treated target nucleic acid molecule is subjected to one or more nucleic acid synthesis reactions. The nucleic acid synthesis reactions can serve to amplify the treated target nucleic acid molecule and/or to modify the form of a nucleic acid molecule. In one embodiment, a treated target nucleic acid molecule or PCR product is transcribed.

Transcription of template DNA such as a target nucleic acid molecule, or an amplified product thereof, can be performed for one strand of the template DNA or for both strands of the template DNA. In one embodiment, the nucleic acid molecule to be transcribed contains a moiety to which an enzyme capable of performing transcription can bind; such a moiety can be, for example, a transcriptional promotor sequence.

Transcription reactions can be performed using any of a variety of methods known in the art, using any of a variety of enzymes known in the art. For example, mutant T7 RNA polymerase (T7 R&DNA polymerase; Epicentre, Madison, Wis.) with the ability to incorporate both dNTPs and rNTPs can be used in the transcription reactions. The transcription reactions can be run under standard reaction conditions known in the art, for example, 40 mM Tris-Ac (pH 7.5), 10 mM NaCl, 6 mM MgCl₂, 2 mM spermidine, 10 mM dithiothreitol, 1 mM of each rNTP, 5 mM of dNTP (when used), 40 nM DNA template, and 5 U/μL T7 R&DNA polymerase, incubating at 37 EC for 2 hours. After transcription, shrimp alkaline phosphatase (SAP) can be added to the cleavage reaction to reduce the quantity of cyclic monophosphate side products. Use of T7 R&DNA polymerase is known in the art, as exemplified by U.S. Pat. Nos. 5,849,546, 6,107,037, and Sousa et al., EMBO J. 14:4609-4621 (1995), Padilla et al., Nucl. Acid Res. 27:1561-1563 (1999), Huang et al., Biochemistry 36:8231-8242 (1997), and Stanssens et al., Genome Res., 14:126-133 (2004).

In addition to transcription with the four regular ribonucleotide substrates (rCTP, rATP, rGTP and rUTP), reactions can be performed replacing one or more ribonucleoside triphosphates with nucleoside analogs, such as those provided herein and known in the art, or with corresponding deoxyribonucleoside triphosphates (e.g., replacing rCTP with dCTP, or replacing rUTP with either dUTP or dTTP). In one embodiment, one or more rNTPs are replaced with a nucleoside or nucleoside analog that, upon incorporation into the transcribed nucleic acid, is not cleavable under the fragmentation conditions applied to the transcribed nucleic acid.

In one embodiment, transcription is performed subsequent to one or more nucleic acid synthesis reactions. For example, transcription of an amplified product can be performed subsequent to amplification of a target nucleic acid molecule. In another embodiment, the treated target nucleic acid molecule is transcribed without any preceding nucleic acid synthesis steps.

In some methods, reactions involving nucleic acids also can include steps in which duplex nucleic acids are denatured to yield single-stranded molecules. Denaturation can be achieved, for example, under conditions in which the temperature of the reaction mixture exceeds that of the melting temperature of a particular duplex nucleic acid.

Numerous nucleic acid reactions, for example, amplification reactions, involve repeated cycles of elevation and reduction of temperature to provide for denaturation and annealing of the strands of nucleic acid hybrids. The apparatus provided in Ser. Nos. 60/372,711, filed Apr. 11, 2002, 60/457,847, filed Mar. 24, 2003, and Ser. No. 10/412,801, filed Apr. 11, 2003, facilitates variation of the temperature of the reaction mixture in a chamber through a direct, rapid and efficient heating and cooling of the relatively low mass and high thermoconductivity of the solid support bottom of the chamber and by avoiding any steps of transferring the reactants into a separate thermocycler instrument.

D. Fragmentation

Once a sufficient quantity of target nucleic acids are generated using known methods, the target nucleic acid sequence can be cleaved into nucleic acid fragments. Any of a variety of methods for cleaving nucleic acid molecules into fragments can be used to generate the nucleic acid fragments. For example, non-specific random fragmentation can be employed. In some cases, the fragmentation method yields a suitable fragment size distribution. Fragmentation of polynucleotides is known in the art and can be achieved in many ways. For example, polynucleotides composed of DNA, RNA, analogs of DNA and RNA, or combinations thereof, can be fragmented physically, chemically, or enzymatically. In one example, physical fragmentation is used to produce random target nucleic acid fragments of various sizes. In another example, partial enzymatic cleavage at one or more specific and/or non-specific cleavage sites can be used to produce the random target nucleic acid fragments utilized herein.

In particular embodiments, fragments of target nucleic acids are prepared for use herein to statistically range in size from among 5-50 bases, 10-40 bases, 11-35 bases, and 12-30 bases. In other embodiments, such as those in which it is contemplated to “trim” the capture oligonucleotide:target-fragment complex prior to the mass spectrometric analysis, the fragments of target nucleic acids can be considerably larger and can statistically range in size from the group of size ranges including=20-50 bases, 30-60 bases, 40-70 bases, 50-80 bases, 60-90 bases, 70-100 bases and higher. Other size ranges contemplated for use herein include between about 50 to about 150 bases, from about 25 to about 75 bases, or from about 12-30 bases. In one particular embodiment, fragments of about 12 to about 30 bases are used. Generally, fragment size range is selected so that shorter fragments bind strongly enough to the capture oligonucleotide and hybridize with sufficient specificity, and longer fragments hybridize with sufficient efficiency so that they are not under-represented. Also, in some embodiments, size range is selected in order to facilitate the desired desorption efficiencies in MALDI-TOF MS.

Fragment size lengths and the range of fragment sizes can be achieved by any of the different fragmentation methods provided herein. For example, when physical fragmentation methods are used, adjustments to the parameters of applying the physical force/strain can result in different fragment sizes and ranges. In another example, when restriction enzymes are used, the number and type of restriction enzymes used and the particular reaction conditions selected can be used to control the average length of fragments generated. Fragments can vary in size, and suitable fragments for use herein are typically less that about 500, less than about 400, less than about 300, less than about 200 nucleotides in length.

In the pool of statistically overlapping fragments, fragments overlap with other fragments; for example, overlapping fragments can overlap with 1 or more, 2 or more, 3 or more, 4 or more, 5 or more, 6 or more, 8 or more, 10 or more, 15 or more, 20 or more other fragments, and typically overlaps with at least 2, at least 3, at least 4, at least 5, at least 6, at least 8, at least 10, at least 15 or at least 20 other fragments.

Overlapping fragments are fragments that have one or more nucleotide positions from the unfragmented target nucleic acid molecule in common. Thus, overlapping fragments include fragments wherein a first fragment contains all nucleotide positions located in a second fragment, plus the first fragment contains additional nucleotide positions, at either the 5′, 3′, or both 5′ and 3′ ends of the first fragment. Overlapping fragments also include fragments where the 3′ end of a first fragment overlaps with the 5′ end of a second fragment. Overlapping fragments need only overlap in one nucleotide position; however, a pool of statistically overlapping fragments also can overlap in at least 2, at least 3, at least 4, at least 5, at least 6, at least 8, at least 10, at least 15, or at least 20 nucleotide positions.

1. Enzymatic Fragmentation of Polynucleotides

Nucleic acid molecule fragments can result from enzymatic cleavage of single or multi-stranded nucleic acid molecules. Multistranded nucleic acid molecules include nucleic acid molecule complexes containing more than one strand of nucleic acid molecules, including for example, double and triple stranded nucleic acid molecules. Depending on the enzyme used, the nucleic acid molecules are cut non-specifically or at specific nucleotide sequences. Any enzyme capable of cleaving a nucleic acid molecule can be used, including but not limited, to endonucleases, exonucleases, single-strand specific nucleases, double-strand specific nucleases, ribozymes, and DNAzymes. A variety of enzymes for fragmenting nucleic acid molecules are known in the art and are commercially available, such as nuclease BAL-31, mung bean nuclease, exonuclease I, exonuclease III, exonuclease VIII, lambda exonuclease, T7 exonuclease, exonuclease T, RecJ, RNase I, RNase III, RNase A, RNase U2, RNase T1, RNase H ShortCut RNase III, Acc I, BasA I, BtgZ I, Mfe I, Sac I, N.BbvC IA, N.BbvC IB, N.BstNBI, I-Ceul, I-Scel, PI-PspI, PI-Scel, McrBC, and other known enzymes (see, e.g., New England Biolabs, Inc. Catalog; Sambrook, J., Russell, D. W., Molecular Cloning: A Laboratory Manual, 3rd ed., Cold Spring Harbor Laboratory Press, Cold Spring Harbor, N.Y., 2001). Enzymes also can be used to degrade large nucleic acid molecules into smaller fragments. The enzymes provided herein can be used alone or in combination to create overlapping target nucleic acid fragments. Generation of overlapping fragments can be achieved by a variety of different methods. For example, a limited/partial digest with a non-specific RNase (RNase I) or a non-specific DNase (DNase I) can be used.

a. Endonuclease Fragmentation

Endonucleases are an exemplary class of enzymes useful for fragmenting nucleic acid molecules. Endonucleases cleave the bonds within a nucleic acid molecule strand. Endonucleases can be specific for either double-stranded or single-stranded nucleic acid molecules. Cleavage can occur randomly within the nucleic acid molecule or at specific sequences. Endonucleases that randomly cleave double-strand nucleic acid molecules often make interactions with the backbone of the nucleic acid molecule. Specific fragmentation of nucleic acid molecules can be accomplished using one or more enzymes in sequential reactions or contemporaneously. Homogenous or heterogenous nucleic acid molecules can be cleaved. Endonucleases also can cleave single-stranded nucleic acids; for example, SI or mung bean nuclease can degrades single-stranded DNA (mung bean) or either DNA or RNA (SI) to yield blunt-ended double-stranded nucleic acid molecules.

Restriction endonucleases are a subclass of endonucleases which recognize specific sequences within double-strand nucleic acid molecules and typically cleave both strands either within or close to the recognition sequence. One commonly used enzyme in DNA analysis is HaeIII, which cuts DNA at the sequence 5′-GGCC-3′. Other exemplary restriction endonucleases include Acc I, Afl III, Alu I, Alw44 I, Apa I, Asn I, Ava I, Ava II, BamH I, Ban II, Bcl I, Bgl I. Bgl II, Bln I, Bsm I, BssH II, BstE II, Cfo I, Cla I, Dde I, Dpn I, Dra I, EcIX I, EcoR I, EcoR I, EcoR II, EcoR V, Hae II, Hae III, Hind III, Hind III, Hpa I, Hpa II, Kpn I, Ksp I, Mlu I, MluN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I. The cleavage sites for these enzymes are known in the art. Also contemplated are Type IIS restriction endonucleases, which cleave downstream from their recognition sites.

Depending on the enzyme used, the cut in the nucleic acid molecule can result in one strand overhanging the other also known as “sticky” ends. For example, BamH I generates cohesive 5′ overhanging ends, and Kpn I generates cohesive 3′ overhanging ends. Alternatively, the cut can result in “blunt” ends that do not have an overhanging end. For example, Dra I cleavage generates blunt ends. Restriction enzymes can cleave nucleic acid molecules containing a particular nucleotide sequence, while not cleaving nucleic acid molecule not containing that nucleotide sequence. In some instances, cleavage recognition sites can be masked by methylation.

Restriction endonucleases can be used to generate a variety of nucleic acid molecule fragment sizes. For example, CviJ I is a restriction endonuclease that recognizes between a two and three base DNA sequence. Complete digestion with CviJ I can result in DNA fragments averaging from 16 to 64 nucleotides in length. Partial digestion with CviJ I can therefore fragment DNA in a “quasi” random fashion similar to shearing or sonication. CviJ I normally cleaves RGCY sites between the G and C leaving readily cloneable blunt ends, wherein R is any purine and Y is any pyrimidine. In the presence of 1 mM ATP and 20% dimethyl sulfoxide the specificity of cleavage is relaxed and CviJ I also cleaves RGCN and YGCY sites. Under these “star” conditions, CviJ I cleavage generates quasi-random digests. Digested or sheared DNA can be size selected at this point.

Methods for using restriction endonucleases to fragment nucleic acid molecules are widely known in the art. In one exemplary protocol a reaction mixture of 20-5011 is prepared containing: DNA 1-3 μg; restriction enzyme buffer 1×; and a restriction endonuclease 2 units for 1 μg of DNA. Suitable buffers also are known in the art and include suitable ionic strength, cofactors, and optionally, pH buffers to provide optimal conditions for enzymatic activity. Specific enzymes can require specific buffers which are generally available from commercial suppliers of the enzyme. An exemplary buffer is potassium glutamate buffer (KGB). Hannish, J. and M. McClelland, “Activity of DNA modification and restriction enzymes in KGB, a potassium glutamate buffer,” Gene Anal. Tech 5:105 (1988); McClelland, M. et al. “A single buffer for all restriction endonucleases,” Nucl. Acids Res. 16:364 (1988). The reaction mixture is incubated at 37 EC for 1 hour or for any time period needed to produce fragments of a desired size or range of sizes. The reaction can be stopped by heating the mixture at 65 EC or 80 EC as needed. Alternatively, the reaction can be stopped by chelating divalent cations such as Mg²⁺ with for example, EDTA.

In particular embodiments, more than one enzyme can be used to fragment the nucleic acid molecule. Multiple enzymes can be used in the same reaction provided the enzymes are active under similar conditions such as ionic strength, temperature, or pH; or, multiple enzymes can be used in sequential reactions. Typically, multiple enzymes are used with a standard buffer such as KGB. When restriction enzymes are used, the nucleic acid molecules can be either partially or completely digested.

DNases also can be used to generate nucleic acid molecule fragments. Anderson, S., “Shotgun DNA sequencing using cloned DNase I-generated fragments,” Nucl. Acids Res. 9:3015-3027 (1981). DNase I (Deoxyribonuclease I) is an endonuclease that non-specifically digests double- and single-stranded DNA into poly- and mono-nucleotides. The enzyme is able to act upon single as well as double-stranded DNA and on chromatin.

Deoxyribonuclease type II is used for many applications in nucleic acid research including DNA sequencing and digestion at an acidic pH. Deoxyribonuclease II from porcine spleen has a molecular weight of 38,000 daltons. The enzyme is a glycoprotein endonuclease with dimeric structure. Optimum pH range is 4.5-5.0 at ionic strength 0.15 M. Deoxyribonuclease II hydrolyzes deoxyribonucleotide linkages in native and denatured DNA yielding products with 3′-phosphates. It also acts on p-nitrophenylphosphodiesters at pH 5.6-5.9. Ehrlich, S. D. et al. “Studies on acid deoxyribonuclease. IX. 5′-Hydroxy-terminal and penultimate nucleotides of oligonucleotides obtained from calf thymus deoxyribonucleic acid,” Biochemistry 10(11):2000-2009 (1971).

Endonucleases can be specific for particular types of nucleic acid molecules. For example, endonuclease can be specific for DNA or RNA, or for single-stranded or double-stranded nucleic acid molecules. Endonucleases can be sequence specific or non-sequence specific. For example, ribonuclease H is an endoribonuclease that specifically degrades the RNA strand in an RNA-DNA hybrid. Ribonuclease A is an endoribonuclease that specifically attacks single-stranded RNA at C and U residues. Ribonuclease A catalyzes cleavage of the phosphodiester bond between the 5′-ribose of a nucleotide and the phosphate group attached to the 3′-ribose of an adjacent pyrimidine nucleotide. The resulting 2′,3′-cyclic phosphate can be hydrolyzed to the corresponding 3′-nucleoside phosphate. RNase T1 digests RNA at only G ribonucleotides, cleaving between the 3′-hydroxy group of a guanylic residue and the 5′-hydroxy group of the flanking nucleotide. RNase U₂ digests RNA at only A ribonucleotides. Examples of base-specific digestion can be found in the publication by Stanssens et al., WO 00/66771.

BenzonaseJ, nuclease P1, and phosphodiesterase I are nonspecific endonucleases that are suitable for generating nucleic acid molecule fragments ranging from 200 base pairs or less. BenzonaseJ (Novagen, Madison, Wis.) is a genetically engineered endonuclease which degrades all forms of DNA and RNA (single stranded, double stranded, linear and circular) and can be used in a wide range of operating conditions. The enzyme completely digests nucleic acids to 5′-monophosphate terminated oligonucleotides 2-5 bases in length. The nucleotide and amino acid sequences for BenzonaseJ is provided in U.S. Pat. No. 5,173,418. Fragmentation of nucleic acids for the methods as provided herein also can be accomplished by dinucleotide (“2 cutter”) or relaxed dinucleotide (“1½ cutter” or “1¼ cutter”) cleavage specificity. Dinucleotide-specific cleavage reagents are known to those of skill in the art (see, e.g., WO 94/21663; Cannistraro et al., Eur. J. Biochem. 181:363-370 (1989); Stevens et al., J. Bacteriol. 164:57-62 (1985); Marotta et al., Biochemistry 12:2901-2904 (1973).

Cleavage using restriction endonucleases can be made partial and/or modified using modified nucleotides that are randomly incorporated into the restriction endonuclease recognition site. These modified nucleotides demonstrate different sensitivity to cleavage relative to standard nucleotides. This different sensitivity can include increased tendency to be cleaved, and also can include decreased tendency to be cleaved, including complete resistance to cleavage. For example, deaza nucleotides, which are resistant to enzymatic cleavage, can be partially and randomly incorporated into the recognition sites for restriction endonucleases, which results in partial cleavage, even though the restriction endonuclease reaction is run to completion. In another example, deoxyuridine can be incorporated into a DNA nucleotide, and uracil-DNA glycosylase can be used to remove the uracil, and the DNA can then be cleaved at this position; thus incorporation of uridine into DNA can show increased tendency to be cleaved. In another example, transcripts of the target nucleic acid molecule of interest can be synthesized with a mixture of regular and α-thio-substrates and the phosphorothioate internucleoside linkages can subsequently be modified by alkylation using reagents such as an alkyl halide (e.g., iodoacetamide, iodoethanol) or 2,3-epoxy-1-propanol. The phosphothioester bonds formed by such modification are not expected to be substrates for RNases. Other exemplary nucleotides that are not cleaved by RNases include 2′fluoro nucleotides, 2′deoxy nucleotides and 2′amino nucleotides. In one example of using this procedure, the cleavage specificity of RNase A can be restricted to CpN or UpN dinucleotides through incorporation of a non-hydrolyzable nucleotide, such as a 2′-modified form of a C nucleotide or U nucleotide, depending on the desired cleavage specificity. Thus, in one example, a transcript (target molecule) can be prepared by incorporating αS-dUTP, αS-ATP, αS-CTP and GTP nucleotides into the transcript. The repertoire of useful dinucleotide-specific cleavage reagents can be further expanded by using additional RNases, such as RNase-U2 and RNase-T1. In the case of a mono-specific RNase, such as RNase-T1, use of non-cleavable nucleotides can limit cleavage of GpN bonds to any three, two or one out of the four possible GpN bonds depending on which nucleotide are selected to be non-cleavable. These selective modification strategies also can be used to prevent cleavage at every base of a homopolymer tract by selectively modifying some of the nucleotides within the homopolymer tract to render the modified nucleotides less resistant or more resistant to cleavage.

b. Exonuclease Fragmentation

Polynucleotides can be fragmented into small polynucleotides using nucleases that remove various lengths of bases from the end of a polynucleotide, termed exonucleases. Exonucleases can fragment double-stranded nucleic acids or can fragment single stranded nucleic acids. An exemplary exonucleases that can fragment either single- or double-stranded nucleic acids is Bal 31 nuclease.

Exonucleases can cleave nucleotides from the ends of a variety of polynucleotides. For example, there are 5′ exonucleases (cleave the DNA from the 5′-end of the DNA chain) and 3′ exonucleases (cleave the DNA from the 3′-end of the chain). Different exonucleases can hydrolyse single-strand or double-strand DNA. For example, Exonuclease III is a 3′ to 5′ exonuclease, releasing 5′-mononucleotides from the 3′-ends of DNA strands; it is a DNA 3′-phosphatase, hydrolyzing 3′-terminal phosphomonoesters; and it is an AP endonuclease, cleaving phosphodiester bonds at apurinic or apyrimidinic sites to produce 5′-termini that are base-free deoxyribose 5′-phosphate residues. In addition, the enzyme has an RNase H activity; it preferentially degrades the RNA strand in a DNA-RNA hybrid duplex, presumably exonucleolytically. In S1, mammalian cells, the major DNA 3′-exonuclease is DNase III (also called TREX-1). Thus, fragments can be formed by using exonucleases to degrade the ends of polynucleotides.

c. Nucleic Acid Enzyme Fragmentation

Catalytic DNA and RNA are known in the art and can be used to cleave nucleic acid molecules to produce nucleic acid molecule fragments. Santoro, S. W. and Joyce, G. F. “A general purpose RNA-cleaving DNA enzyme,” Proc. Natl. Acad. Sci. USA 94:4262-4266 (1997). DNA as a single-stranded molecule can fold into three dimensional structures similar to RNA, and the 2′-hydroxy group is dispensable for catalytic action. As ribozymes, DNAzymes also can be made, by selection, to depend on a cofactor. This has been demonstrated for a histidine-dependent DNAzyme for RNA hydrolysis. U.S. Pat. Nos. 6,326,174 and 6,194,180 disclose deoxyribonucleic acid enzymes, catalytic and enzymatic DNA molecules, capable of cleaving nucleic acid sequences or molecules, particularly RNA.

The use of ribozymes for cleaving nucleic acid molecules is known in the art. Ribozymes are RNAs that catalyze a chemical reaction, e.g., cleavage of a covalent bond. Uhlenbeck demonstrated a small active ribozyme, the hammerhead ribozyme, in which the catalytic and substrate strands were separated (Uhlenbeck, Nature 328:596-600 (1987)). Such ribozymes bind substrate RNAs through base-pairing interactions, cleave the bound target RNA, release the cleavage products, and are recycled so that they can repeat this process multiple times. Haseloff and Gerlach enumerated general design rules for simple hammerhead ribozymes capable of acting in trans (Haseloffet al., Nature, 334:585-591 (1988)). A variety of different hammerhead ribozymes with high cleavage specificity have been developed, and general approaches for design of hammerhead ribozymes having desired substrate specificity are known in the art, as exemplified by U.S. Pat. Nos. 5,646,020 and 6,096,715. Another type if ribozyme with trans-cleavage activity are the δ ribozymes derived from the genome of hepatitis δ virus. Ananvoranich and Perrault have described the factors for substrate specificity of δ ribozyme cleavage (Ananvoranich et al., J. Biol. Chem. 273:13812-13188 (1998)). Hairpin ribozymes also can be used for trans-cleavage, and the principles for substrate specificity for hairpin ribozymes also are known (see, e.g., Perez-Ruiz et al., J. Biol. Chem. 274:29376-29380 (1999)). One skilled in the art can use the known principles of substrate specificity to select the ribozyme and design the ribozyme sequence to achieve the desired nucleic acid molecule cleavage specificity.

A DNA nickase, or DNase, can be used to recognize and cleave one strand of a DNA duplex. Numerous nickases are known. Among these, for example, are nickase NY2A nickase and NYS1 nickase (Megabase) with the following cleavage sites:
NY2A: 5′ . . . R AG . . . 3′
   3′ . . . Y TC . . . 5′ where R=A or G and Y═C or T
NYS1: 5′ . . . CC[A/G/T] . . . 3′
   3′ . . . GG[T/C/A] . . . 5′.
      Subsequent chemical treatment of the products from the nickase reaction results in the cleavage of the phosphate backbone and the generation of fragments.

The Fen-1 fragmentation method involves the enzymes Fen-1 enzyme, which is a site-specific nuclease known as a “flap” endonuclease (U.S. Pat. Nos. 5,843,669, 5,874,283, and 6,090,606). This enzyme recognizes and cleaves DNA “flaps” created by the overlap of two oligonucleotides hybridized to a target DNA strand. This cleavage is highly specific and can recognize single base variations, permitting detection of a single methylated base at a nucleotide locus of interest. Fen-1 enzymes can be Fen-1 like nucleases e.g., human, murine, and Xenopus XPG enzymes and yeast RAD2 nucleases or Fen-1 endonucleases from, for example, M. jannaschii, P. furiosus, and P. woesei.

Another technique that can be used is cleavage of DNA chimeras. Tripartite DNA-RNA-DNA probes are hybridized to target nucleic acid molecules, such as M. tuberculosis-specific sequences. Upon the addition of RNase H, the RNA portion of the chimeric probe is degraded, releasing the DNA portions (Yule, Bio/Technology 12:1335 (1994)).

d. Base-Specific Fragmentation

Target nucleic acid molecules can be fragmented using nucleases that selectively cleave at a particular base (e.g., A, C, T or G for DNA and A, C, U or G for RNA) or base type (i.e., pyrimidine or purine). In one embodiment, RNases that specifically cleave 3 RNA nucleotides (e.g., U, G and A), 2 RNA nucleotides (e.g., C and U) or 1 RNA nucleotide (e.g., A), can be used to base specifically cleave transcripts of a target nucleic acid molecule. For example, RNase T1 cleaves ssRNA (single-stranded RNA) at G ribonucleotides, RNase U2 digests ssRNA at A ribonucleotides, RNase CL3 and cusativin cleave ssRNA at C ribonucleotides, PhyM cleaves ssRNA at U and A ribonucleotides, and RNase A cleaves ssRNA at pyrimidine ribonucleotides (C and U). The use of mono-specific RNases such as RNase T₁ (G specific) and RNase U₂ (A specific) is known in the art (Donis-Keller et al., Nucleic Acids Res. 4:2527-2537 (1977); Gupta and Randerath, Nucleic Acids Res. 4:1957-1978 (1977); Kuchino and Nishimura, Methods Enzymol. 180:154-163 (1989); and Hahner et al., Nucl. Acids Res. 25(10):1957-1964 (1997)). Another enzyme, chicken liver ribonuclease (RNase CL3) has been reported to cleave preferentially at cytidine, but the enzyme's proclivity for this base has been reported to be affected by the reaction conditions (Boguski et al., J. Biol. Chem. 255:2160-2163 (1980)). Reports also claim cytidine specificity for another ribonuclease, cusativin, isolated from dry seeds of Cucumis sativus L (Rojo et al., Planta 194:328-338 (1994)). Alternatively, the identification of pyrimidine residues by use of RNase PhyM (A and U specific) (Donis-Keller, H. Nucleic Acids Res. 8:3133-3142 (1980)) and RNase A (C and U specific) (Simoncsits et al., Nature 269:833-836 (1977); Gupta and Randerath, Nucleic Acids Res. 4:1957-1978 (1977)) has been demonstrated. Examples of such cleavage patterns are given in Stanssens et al., WO 00/66771.

In addition, bases can be targeted, for example, by incorporating a modified nucleotide into the nucleic acid, and excising the base of the nucleotide; subsequent treatment of the nucleic acid under the appropriate conditions or with an enzyme, can result in fragmentation of the nucleic acid at the site of the excised base. For example, dUTP can be incorporated into DNA, and base specific fragmentation can be accomplished by removing the uracil base using UDG, and subsequently cleaving the DNA under known cleavage conditions. In another example, methyl-cytosine can be incorporated into DNA, and base specific fragmentation can be accomplished using methyl cytosine deglycosylase to remove the methyl cytosine, followed by treatment under known conditions to result in DNA fragmentation. Base-specific fragmentation can be used in partial cleavage reactions (including partial cleavage reactions performed to completion when the target nucleic acid molecules contain non-cleavable nucleotides incorporated therein), and total cleavage reactions.

Base specific cleavage reaction conditions using an RNase are known in the art, and can include, for example 4 mM Tris-Ac (pH 8.0), 4 mM KAc, 1 mM spermidine, 0.5 mM dithiothreitol and 1.5 mM MgCl₂.

In one embodiment, amplified product can be transcribed into a single stranded RNA molecule and then cleaved base specifically by an endoribonuclease. In one embodiment, transcription of a target nucleic acid molecule can yield an RNA molecule that can be cleaved using specific RNA endonucleases. For example, base specific cleavage of the RNA molecule can be performed using two different endoribonucleases, such as RNase T1 and RNase A. RNase T1 specifically cleaves G nucleotides, and RNase A specifically cleaves pyrimidine ribonucleotides (i.e., cytosine and uracil residues). In one embodiment, when an enzyme that cleaves more than one nucleotide, such as RNase A, is used for cleavage, non-cleavable nucleosides, such as dNTP's can be incorporated during transcription of the target nucleic acid molecule or amplified product. For example, dCTPs can be incorporated during transcription of the amplified product, and the resultant transcribed nucleic acid can be subject to cleavage by RNase A at U ribonucleotides, but resistant to cleavage by RNase A at C deoxyribonucleotides. In another example, dTTPs can be incorporated during transcription of the target nucleic acid molecule, and the resultant transcribed nucleic acid can be subject to cleavage by RNase A at C ribonucleotides, but resistant to cleavage by RNase A at T deoxyribonucleotides. By selective use of non-cleavable nucleosides such as dNTPs, and by performing base specific cleavage using RNases such as RNase A and RNase T1, base cleavage specific to three different nucleotide bases can be performed on the different transcripts of the same target nucleic acid sequence. For example, the transcript of a particular target nucleic acid molecule can be subjected to G-specific cleavage using RNase T1; the transcript can be subjected to C-specific cleavage using dTTP in the transcription reaction, followed by digestion with RNase A; and the transcript can be subjected to T-specific cleavage using dCTP in the transcription reaction, followed by digestion with RNase A.

In another embodiment, the use of dNTPs, different RNases, and both orientations of the target nucleic acid molecule can allow for six different cleavage schemes. For example, a double stranded target nucleic acid molecule can yield two different single stranded transcription products, which can be referred to as a transcript product of the forward strand of the target nucleic acid molecule and a transcript product of the reverse strand of the target nucleic acid molecule. Each of the two different transcription products can be subjected to three separate base specific cleavage reactions, such as G-specific cleavage, C-specific cleavage and T-specific cleavage, as described herein, to result in six different base specific cleavage reactions. The six possible cleavage schemes are listed in Table 1. Use of four different base specific cleavage reactions can yield information on all four nucleotide bases of one strand of the target nucleic acid molecule. By taking into account that cleavage of the forward strand can be mimicked by cleaving the complementary base on the reverse strand, base specific cleavage can be achieved for each of the four nucleotides of the forward strand by reference to cleavage of the reverse strand. For example, the three base-specific cleavage reactions can be performed on the transcript of the target nucleic acid molecule forward strand, to yield G-, C- and T-specific cleavage of the target nucleic acid molecule forward strand; and a fourth base specific cleavage reaction can be a T-specific cleavage reaction of the transcript of the target nucleic acid molecule reverse strand, the results are equivalent to A-specific cleavage of the transcript of the target nucleic acid molecule forward strand. One skilled in the art appreciates that base specific cleavage to yield information on all four nucleotide bases of one target nucleic acid molecule strand can be accomplished using a variety of different combinations of possible base specific cleavage reactions, including cleavage reactions provided in Table 1 for RNases T1 and A, and additional cleavage reactions for forward or reverse strands and/or using non-hydrolyzable nucleotides can be performed with other base specific RNases known in the art or disclosed herein.

TABLE 1

| | Forward Primer | Reverse Primer |
|---|---|---|
| RNase T1 | G specific cleavage | G specific cleavage |
| RNase A; dCTP | T specific cleavage | T specific cleavage |
| RNase A; dTTP | C specific cleavage | C specific cleavage |

In one example, RNase U2 can be used to base specifically cleave target nucleic acid molecule transcripts. RNase U2 can base specifically cleave RNA at A nucleotides. Thus, by use of RNases T1, U2 and A, and by use of the appropriate dNTPs (in conjunction with use of RNase A), all four base positions of a target nucleic acid molecule can be examined by base specifically cleaving transcript of only one strand of the target nucleic acid molecule. In some embodiments, non-cleavable nucleoside triphosphates are not required when base specific cleavage is performed using RNases that base specifically cleave only one of the four ribonucleotides. For example, use of RNase T1, RNase CL3, cusativin, or RNase U2 for base specific cleavage does not require the presence of a non-cleavable nucleotides in the target nucleic acid molecule transcript. Use of RNases such as RNase T1 and RNase U2 can yield information on all four nucleotide bases of a target nucleic acid molecule. For example, transcripts of both the forward and reverse strands of a target nucleic acid molecule or amplified product can be synthesized, and each transcript can be subjected to base specific cleavage using RNase T1 and RNase U2. The resulting cleavage pattern of the four cleavage reactions yield information on all four nucleotide bases of one strand of the target nucleic acid molecule. In such an embodiment, two transcription reactions can be performed: a first transcription of the forward target nucleic acid molecule strand and a second of the reverse target nucleic acid molecule strand.

Also contemplated for use in the methods are a variety of different base specific cleavage methods. A variety of different base specific cleavage methods are known in the art and are described herein, including enzymatic base specific cleavage of RNA, enzymatic base specific cleavage of modified DNA, and chemical base specific cleavage of DNA. For example enzymatic base specific cleavage, such as cleavage using uracil-deglycosylase (UDG) or methylcytosine deglycosylase (MCDG), are known in the art and described herein, and can be performed in conjunction with the enzymatic RNase-mediated base specific cleavage reactions described herein. Further contemplated herein is the use of base-specific cleavage reactions to fragment nucleic acids such as RNA that contain non-hydrolyzable bases, thus resulting in a partially complete base specific cleavage reaction.

2. Physical Fragmentation of Polynucleotides

Fragmentation of nucleic acid molecules can be achieved using physical or mechanical forces including mechanical shear forces and sonication. Physical fragmentation of nucleic acid molecules can be accomplished, for example, using hydrodynamic forces. Typically nucleic acid molecules in solution are sheared by repeatedly drawing the solution containing the nucleic acid molecules into and out of a syringe equipped with a needle. Thorstenson, Y. R. et al. “An Automated Hydrodynamic Process for Controlled, Unbiased DNA Shearing,” Genome Research 8:848-855 (1998); Davison, P. F. Proc. Natl. Acad. Sci. USA 45:1560-1568 (1959); Davison, P. F. Nature 185:918-920 (1960); Schriefer, L. A. et al. “Low pressure DNA shearing: a method for random DNA sequence analysis,” Nucl. Acids Res. 18:7455-7456 (1990). Shearing of DNA, for example with a hypodermic needle, typically generates a majority of fragments ranging from 1-2 kb, although a minority of fragments can be as small as 300 bp.

Devices for shearing nucleic acid molecules, including for example genomic DNA, are commercially available. An exemplary device uses a syringe pump to create hydrodynamic shear forces by pushing a DNA sample through a small abrupt contraction. Thorstenson, Y. R. et al. “An Automated Hydrodynamic Process for Controlled, Unbiased DNA Shearing,” Genome Research 8:848-855 (1998). The volume for shearing is typically 100-250 μL, and processing time to less than 15 minutes. Shearing of the samples can be completely automated by computer control.

The hydrodynamic point-sink shearing method developed by Oefner et al. is one method of shearing nucleic acid molecules that utilizes hydrodynamic forces. Oefner, P. J. et al. “Efficient random subcloning of DNA sheared in a recirculating point-sink flow system,” Nucl. Acids Res. 24(20):3879-3886 (1996). “Point-sink” refers to a theoretical model of the hydrodynamic flow in this system. The rate-of-strain tensor describes the force on a molecule and therefore, its breakage. DNA breakage was attributed to the “shearing” terms of this tensor, and this class of method of fragmenting was referred to as shearing. Breakage can be caused by both the shearing terms (when the fluid is inside the narrow tube or orifice) and the extensional strain terms (when the fluid approaches the orifice). Point-sink shearing is accomplished by forcing nucleic acid molecules, for example DNA, through a very small diameter tubing by applying pressure with a pump, for example a HPLC pump. The resulting fragments have a tight size range with the largest fragments being about twice as long as the smallest fragments. The size of the fragments are inversely proportional to the flow rate.

Nucleic acid molecule fragments also can be obtained by agitating large nucleic acid molecules in solution, for example by mixing, blending, stirring, or vortexing the solution. Hershey, A. D. and Burgi, E. J. Mol. Biol. 2:143-152 (1960); Rosenberg, H. S. and Bendich, A. J. Am. Chem. Soc. 82:3198-3201 (1960). The solution can be agitated for various lengths of time until fragments of a desired size or range of sizes are obtained. The addition of beads or particles to the solution can assist in fragmenting the nucleic acid molecules.

One suitable method of physically fragmenting nucleic acid molecules is based on sonicating the nucleic acid molecule. Deininger, P. L. “Approaches to rapid DNA sequence analysis,” Anal. Biochem. 129:216-223 (1983). The generation of nucleic acid molecule fragments by sonication is typically performed by placing a microcentrifuge tube containing buffered nucleic acid molecules into an ice-water bath in a sonicator, for example a cup-horn sonicator, and sonicating for a varying number of short bursts using maximum output and continuous power. The short bursts can be about 10 seconds in duration. See for example Bankier, A. T. et al. “Random cloning and sequencing by the M13/dideoxynucleotide chain termination method,” Meth. Enzymol. 155:51-93 (1987). In one exemplary sonication protocol, sonication of large nucleic acid molecules resulted in fragments in the range of 300-500 bp or 2-10 kb depending on conditions of sonication such as duration and sonication intensity. Kawata, Y. et al. “Preparation of a Genomic Library Using TA Vector,” Prep. Biochem & Biotechnol. 29(1):91-100 (1999).

During sonication, temperature increases can result in uneven fragment distribution patterns, and for that reason, the temperature of the bath can be monitored carefully, and fresh ice-water can be added when necessary. An exemplary sonication protocol to determine specific conditions for sonication includes distributing approximately 100 μg of nucleic acid molecule sample, in 350 μl of a suitable buffer, into ten aliquots of 35 μl, five of which are subjected to sonication for increasing numbers of 10 second bursts. The nucleic acid molecule samples are cooled by placing the tubes in an ice-water bath for at least 1 minute between each 10 second burst. The ice-water bath in the sonicator can be replaced between each sample as needed. The samples can be centrifuged to reclaim condensation and an aliquot electrophoresed on a agarose gel versus a size marker. Based on the fragment size ranges detected from agarose gel electrophoresis, the remaining 5 tubes can be sonicated accordingly to obtain the desired fragment sizes.

Fragmentation of nucleic acid molecules also can be achieved using a nebulizer. Bodenteich, A., Chissoe, S., Wang, Y.-F. and Roe, B. A. (1994) In Adams, M. D., Fields, C. and Venter, J. C. (eds) Automated DNA Sequencing and Analysis, Academic Press, San Diego, Calif. Nebulizers are known in the art and commercially available. An exemplary protocol for nucleic acid molecule fragmentation using a nebulizer includes placing 2 ml of a buffered nucleic acid molecule solution (approximately 50 μg) containing 25-50% glycerol in an ice-water bath and subjecting the solution to a stream of gas, for example nitrogen, at a pressure of 8-10 psi for 2.5 minutes. It is appreciated that any gas can be used, particularly inert gases. Gas pressure is the primary determinant of fragment size. Varying the pressure can produce various fragment sizes. Use of an ice-water bath for nebulization can be used to generate evenly distributed fragments. Similarly, fragments can be generated using a high pressure spray atomizer. Cavalieri, L. F. and Rosenberg, B. H., J. Am. Chem. Soc. 81:5136-5139 (1959).

Another method for fragmenting nucleic acid molecule employs repeatedly freezing and thawing a buffered solution of nucleic acid molecules. The sample of nucleic acid molecules can be frozen and thawed as necessary to produce fragments of a desired size or range of sizes. Additionally, nucleic acid molecules can be bombarded with ions or particles to generate fragments of various sizes. For example, nucleic acid molecules can be exposed to an ion extraction beamline under vacuum. Ions are extracted from an electron beam ion trap at 7 kV*q and directed onto the target nucleic acid molecules. The nucleic acid molecules can be irradiated for any length of time, typically for a few hours until, for example, a total fluence of 100 ions/μm² is achieved.

Nucleic acid molecule fragmentation also can be achieved by irradiating the nucleic acid molecules. Typically, radiation such as gamma or x-ray radiation is sufficient to fragment the nucleic acid molecules. The size of the fragments can be adjusted by adjusting the intensity and duration of exposure to the radiation. Ultraviolet radiation also can be used. The intensity and duration of exposure also can be adjusted to minimize undesirable effects of radiation on the nucleic acid molecules.

Boiling nucleic acid molecules also can produce fragments. Typically a solution of nucleic acid molecules is boiled for a couple hours under constant agitation. Fragments of about 500 bp can be achieved. The size of the fragments can vary with the duration of boiling.

3. Chemical Fragmentation of Nucleic Acid Molecules

Chemical fragmentation can be used to fragment nucleic acid molecules either with base specificity or without base specificity. Nucleic acid molecules can be fragmented by chemical reactions including for example, hydrolysis reactions including base and acid hydrolysis. Alkaline conditions can be used to fragment nucleic acid molecules containing nicks or RNA because RNA (or unpaired bases) is unstable under alkaline conditions. See Nordhoffet al. “Ion stability of nucleic acids in infrared matrix-assisted laser desorption/ionization mass spectrometry,” Nucl. Acids Res. 21(15):3347-3357 (1993). DNA can be hydrolyzed in the presence of acids, typically strong acids such as 6M HCl. The temperature can be elevated above room temperature to facilitate the hydrolysis. Depending on the conditions and length of reaction time, the nucleic acid molecules can be fragmented into various sizes including single base fragments. Hydrolysis can, under rigorous conditions, break both of the phosphate ester bonds and also the N-glycosidic bond between the deoxyribose and the purines and pyrimidine bases.

An exemplary acid/base hydrolysis protocol for producing nucleic acid molecule fragments are known (see, e.g., Sargent et al. Meth. Enz 152:432 (1988)). Briefly, 1 g of DNA is dissolved in 50 mL 0.1 N NaOH. 1.5 mL concentrated HCl is added, and the solution is mixed quickly. DNA precipitates immediately, and should not be stirred for more than a few seconds to prevent formation of a large aggregate. The sample is incubated at room temperature for 20 minutes to partially depurinate the DNA. Subsequently, 2 mL 10 N NaOH(OH— concentration to 0.1 N) is added, and the sample is stirred until DNA redissolves completely. The sample is then incubated at 65 EC for 30 minutes to hydrolyze the DNA. Typical sizes range from about 250-1000 nucleotides but can vary lower or higher depending on the conditions of hydrolysis.

Chemical cleavage also can be specific. For example, selected nucleic acid molecules can be cleaved via alkylation, particularly phosphorothioate-modified nucleic acid molecules (see, e.g., K. A. Browne, “Metal ion-catalyzed nucleic Acid alkylation and fragmentation,” J. Am. Chem. Soc. 124(27):7950-7962 (2002)). Alkylation at the phosphorothioate modification renders the nucleic acid molecule susceptible to cleavage at the modification site. I. G. Gut and S. Beck describe methods of alkylating DNA for detection in mass spectrometry. I. G. Gut and S. Beck, “A procedure for selective DNA alkylation and detection by mass spectrometry,” Nucl. Acids Res. 23(8):1367-1373 (1995).

Various additional chemicals and methods for base-specific and base non-specific chemical cleavage of oligonucleotides are known in the art, and are contemplated for use in the fragmentation methods provided herein. For example, base-specific cleavage can be accomplished using chemicals such as piperidine formate, piperidine, dimethyl sulfate, hydrazine and sodium chloride, hydrazine. For example, DNA can be base-specifically cleaved at G nucleotides using dimethyl sulfate and piperidine; DNA can be base-specifically cleaved at A and G nucleotides using dimethyl sulfate, piperidine and acid; DNA can be base-specifically cleaved at C and T nucleotides using hydrazine and piperidine; DNA can be base-specifically cleaved at C nucleotides using hydrazine, piperidine and sodium chloride; and DNA can be base-specifically cleaved at A nucleotides, with a lower specificity for C nucleotides using a strong base. In another example, ribonucleotides and deoxyribonucleotides can be incorporated into a target nucleic acid molecule, and the target nucleic acid can be contacted with conditions for specifically cleaving either RNA or DNA, resulting in base specific cleavage (either partial or complete cleavage) according to the composition of the target nucleic acid molecule.

4. Combinations of Fragmentation Methods

Fragments also can be formed using any combination of fragmentation methods described herein, using e.g., a combination of different enzymatic fragmentation methods, a combination of different chemical fragmentation methods, a combination of different physical fragmentation methods, or enzymatic and chemical fragmentation methods, enzymatic and physical fragmentation methods, chemical and physical fragmentation methods, or enzymatic and chemical and physical fragmentation methods. A few specific examples include, but are not limited to, a combination of different base-specific cleavage methods, and a combination of shearing with a sequence-specific enzyme. Methods for producing specific fragments can be combined with methods for producing random fragments. Further, different methods for producing random fragments can be combined, and different methods for producing specific fragments can be combined. For example, one or more enzymes that cleave a nucleic acid molecule at a specific site can be used in combination with one or more enzymes that specifically cleave the nucleic acid molecule at a different site. In another example, enzymes that cleave specific kinds of nucleic acid molecules can be used in combination, for example, an RNase in combination with a DNase or a single-strand specific nuclease can be used in combination with a double-strand specific nuclease, or an exonuclease can be used in combination with an endonuclease. In still another example, an enzyme that cleaves nucleic acid molecules randomly can be used in combination with an enzyme that cleaves nucleic acid molecules specifically. Use of fragmentation in combination refers to performing one or more methods after another or contemporaneously, on a nucleic acid molecule.

As contemplated herein, use in combination also can encompass using a first fragmentation method on a first fraction of a nucleic acid molecule sample, using a second fragmentation method on a second fraction of the nucleic acid molecule sample. The two samples can be separately analyzed in subsequent detection and mass measurement methods, or the two samples can be pooled together and simultaneously analyzed in subsequent detection and mass measurement methods. Combinations of fragmentation methods can include 2 or more fragmentation methods, 3 or more fragmentation methods, or 4 or more fragmentation methods.

5. Fragmentation after Hybridization

Target nucleic acids also can be fragmented after the target nucleic acid has hybridized with a capture oligonucleotide probe. In one embodiment, the target nucleic acids undergo one or more fragmentation steps prior to hybridizing with a capture oligonucleotide probe, and then undergo one or more additional fragmentation steps after hybridizing with a capture oligonucleotide probe. In another embodiment, the target nucleic acids do not undergo any fragmentation steps prior to hybridizing with a capture oligonucleotide probe, but undergo one or more fragmentation steps after hybridizing with a capture oligonucleotide probe. Examples of reactions that occur after the target nucleic acid hybridizes to the capture oligonucleotide probe include enzymatic and chemical fragmentation. In one embodiment, such a post-hybridization fragmentation step selectively fragments single-stranded nucleic acids but not double-stranded nucleic acids. In another embodiment, post-hybridization fragmentation includes base-specific cleavage.

E. Capture Oligonucleotide

Also included in the methods and compositions provided herein are one or more capture oligonucleotides to which target nucleic acid fragments can hybridize. A capture oligonucleotide provided herein can be contacted with target nucleic acid fragments under conditions in which, typically, some target nucleic acid fragments hybridize to capture oligonucleotide, and some target nucleic acid fragments do not hybridize to capture oligonucleotide. Target nucleic acid fragments that hybridize to a capture oligonucleotide can be separated from target nucleic acid fragments that do not hybridize to a capture oligonucleotide. Target nucleic acid fragments that hybridize to a capture oligonucleotide and target nucleic acid fragments that do not hybridize to a capture oligonucleotide can be subjected to separate treatment steps after contacting the capture oligonucleotide and/or after separating hybridized and unhybridized fragments. After the contacting the target nucleic acid fragments with the capture oligonucleotide, the mass of target nucleic acid fragments can be measured. Since contacting the target nucleic acid fragments with a capture oligonucleotide can result in a separation of nucleic acid fragments, mass spectra from capture oligonucleotide-contacted target nucleic acid fragments can have fewer masses (e.g., fewer peaks at different masses) relative to fragments not contacted with a capture oligonucleotide. While capture oligonucleotides can be used to hybridize to only a single sequence, it is contemplated herein that capture oligonucleotides also can be used for intentionally hybridizing with more than one capture oligonucleotide sequence by using, for example, degenerate bases, or low or medium stringency hybridization conditions. The number and variety of different target nucleic acid fragments that hybridize to the capture oligonucleotide can determine the number and variety of different fragments measured by mass spectrometry.

Thus, one exemplary method provided herein is a method for measuring the mass of target nucleic acid fragments, comprising:

(a) controlling the complexity of target nucleic acid fragments hybridized to a capture oligonucleotide probe, wherein each of the target nucleic acid fragments contains at least a first region that hybridizes to the capture oligonucleotide probe; and

(b) measuring the mass of the target nucleic acid fragments hybridized to the capture oligonucleotide probe using mass spectrometry;

wherein the step of controlling the complexity includes modulating the number of different sequences in the first region of the target nucleic acid fragments that hybridize to the capture oligonucleotide probe, whereby two or more target nucleic acid fragments containing different nucleotide sequences in the respective first regions hybridize to the capture oligonucleotide probe.

1. Controlling complexity of Target Nucleic Acid Fragments

The methods provided herein include a step of measuring the mass of target nucleic acid fragments, as described elsewhere herein. Depending on the number and/or variability of the target nucleic acid fragments whose mass is measured in a particular assay (e.g., whose mass is measured in a single mass spectrum), the masses of different fragments may or may not be easily distinguishable, the number of different nucleotide sequences represented in a particular mass can be large or small, and absent masses (e.g., possible but not present mass peak) may or may not be easily identified. When fragment complexity is extremely low, a mass spectrum has only a few present/absent masses, which can limit the degree of robustness provided by the method of sequence determination (e.g., when only a single fragment is determined by mass measurement to be present or absent, little information is provided that is not already obtainable in traditional sequencing by hybridization methods). When fragment complexity is extremely high, a mass spectrum can have a large number of present/absent masses and each mass can represent many different nucleotide sequences, which can limit the extent that a particular observation (e.g., mass present or absent) can be used to assign a nucleotide sequence with high probability (e.g., when too many fragments can be present/absent, little decrease in complexity is provided that is different from mass spectrometric methods without capture oligonucleotide hybridization). Thus, controlling the complexity of target nucleic acid fragments can serve to “tune” a mass spectrum such that a mass spectrum can provide a large number of resolvable observations (e.g., resolvable presence or absence of a mass), and, optionally, the observations represent a small enough number of different sequences that permit sequence determination.

In one embodiment, the complexity of the target nucleic acid fragments is controlled prior to measuring the mass of the target nucleic acid fragments. In another embodiment, controlling the complexity includes controlling one region of a target nucleic acid fragment, where at least some target nucleic acid fragments further contain a second region for which the complexity is not controlled or the complexity is differently controlled.

a. Methods of Controlling Complexity

As contemplated herein, fragmentation of the target nucleic acids, together with hybridization of the target nucleic acids with capture oligonucleotides attached to a solid support, can serve to control or to reduce the complexity of the mixture of target nucleic acids whose mass is to be analyzed.

In an example of controlling complexity, fragmentation controls the length of the target nucleic acid fragments, and also can control a portion of the sequence in the target nucleic acid fragments, including the identity of one or more nucleotide positions at the 3′, 5′, or both 3′ and 5′ ends of the target nucleic acid fragments. In another example, hybridization of the target nucleic acids to the capture oligonucleotides can control the complexity of the target nucleic acid sequence in the region that hybridizes with the capture oligonucleotide probe. In one embodiment, when a first region of a target nucleic acid hybridizes with a capture oligonucleotide probe, the complexity of the first region of the target nucleic acid can be controlled separately from the complexity of a second, non-hybridizing region of the target nucleic acid.

For example, when a capture probe is 5 nucleotides long, and target nucleic acid sequences are 8 nucleotides long, the complexity can be controlled using, for example, hybridization conditions and a capture oligonucleotide probe sequence that permits only two different target nucleic acid sequences to hybridize to the capture oligonucleotide probe sequence, resulting in the possible number of different target nucleic acid fragments that hybridize to a particular capture probe oligonucleotide being limited to no more than 512. The complexity can be further limited using sequence-specific fragmentation conditions such as using a sequence-specific endonuclease or base-specific cleavage, as discussed above.

Generally, the complexity of both hybridizing and non-hybridizing regions of target nucleic acid fragments hybridized to a capture oligonucleotide probe can be controlled by controlling the length of the target nucleic acid fragments, controlling the number of different lengths in the statistical size range of target nucleic acid fragments, controlling the overall length of the target nucleic acid being analyzed, using sequence-specific or non-specific fragmentation methods, and controlling the ability of a capture oligonucleotide probe to hybridize with the nucleotide positions at either the 5′ or 3′ ends of the target nucleic acid fragments. In addition, the complexity of the hybridizing region can further be controlled by modifying the conditions under which the target nucleic acids are exposed to the capture oligonucleotide (e.g., low stringency hybridization conditions, medium stringency hybridization conditions, or high stringency hybridization conditions), and by modifying the number of nucleotides and/or degeneracy of the nucleotides of the capture oligonucleotide probe (e.g., by using universal or semi-universal nucleotides). For example, the complexity of target nucleic acid fragment hybridized to a capture oligonucleotide probe can be decreased by decreasing the length of target nucleic acid fragments, decreasing the number of different lengths in the statistical size range of target nucleic acid fragments, decreasing the overall length of the target nucleic acid being analyzed, using sequence-specific or base-specific fragmentation methods, using a capture oligonucleotide probe that favors hybridization with the nucleotide positions at either the 5′ or 3′ ends of the target nucleic acid fragments, using increased stringency hybridization conditions, and including more, sequence-specific nucleotides in the capture oligonucleotide. In another example, the complexity of both hybridizing and non-hybridizing regions of target nucleic acid fragments hybridized to a capture oligonucleotide probe can be increased by increasing the length of the target nucleic acid fragments, increasing the number of different lengths in the statistical size range of target nucleic acid fragments, increasing the overall length of the target nucleic acid being analyzed, using non-specific fragmentation methods, using a capture oligonucleotide probe that does not favor hybridization with a particular region of the target nucleic acid, using decreased stringency hybridization conditions, and including fewer and/or less sequence-specific nucleotides (e.g., universal or semi-universal bases) in the capture oligonucleotide.

In one embodiment, the complexity of the target nucleic acid fragments that hybridize to a capture oligonucleotide probe is controlled prior to the step of measuring the mass of the target nucleic acid fragments. For example, controlling the complexity of target nucleic acid fragments can be carried out prior to hybridizing the target nucleic acid fragments to the capture oligonucleotide probes (e.g., in a fragmentation step), and/or controlling the complexity of target nucleic acid fragments can include hybridizing the target nucleic acid fragments to the capture oligonucleotide probes, and/or controlling the complexity of target nucleic acid fragments can be carried out after hybridizing the target nucleic acid fragments to the capture oligonucleotide probes, but before measuring the mass of the target nucleic acid fragments (e.g., in subsequent fragmentation steps such as “trimming”).

Target nucleic acid fragmentation products can be captured onto a solid-phase in a variety of ways. For example, capture oligonucleotides that specifically or semi-specifically hybridize with one or more fragmentation products can be attached to a solid support for either specific or “semi-specific” capture of the product.

One skilled in the art can, according to the teachings provided herein and the knowledge in the art, estimate the expected complexity of target nucleic acid fragments bound to a particular capture oligonucleotide. As an example, where a capture oligonucleotide containing a particular sequence contains a single degenerate position comprising a universal nucleotide (e.g., Inosine), up to four different target nucleic acid fragments of the same length as the capture oligonucleotide and same sequence composition (except for the nucleotide at the position complementary to the universal base) could bind to that particular capture oligonucleotide with roughly equal binding affinity. If larger target nucleic acid fragments also are present and are from 1 to 5 nucleotides longer than the capture oligonucleotide, then up to 30,948 different target nucleic acid fragments could bind to a single capture oligonucleotide sequence (see FIG. 2). Similarly, where a capture oligonucleotide has 2 degenerate positions therein corresponding to universal oligonucleotides, up to 16 different target nucleic acid fragments of the same length and sequence composition (except for the nucleotides at the position complementary to the universal bases) could bind to that particular capture oligonucleotide with roughly equal binding affinity.

In one embodiment, the non-hybridizing regions of the target nucleic acid fragments can be completely removed. This can be accomplished, for example, by creating target nucleic acid fragments of the same size as the capture oligonucleotide probes, or by creating target nucleic acid fragments larger than the capture oligonucleotide probes, hybridizing the target nucleic acids to the capture oligonucleotide probes and then cleaving the non-hybridized nucleotides using a single-strand-specific nuclease.

In some embodiments, information regarding the minimum number of different sequences that hybridize to a particular capture probe can be obtained. For example, when low stringency hybridization conditions or degenerate capture oligonucleotide probes are used, more than one target nucleic acid sequence can hybridize to the same capture oligonucleotide probe sequence. If, in such a case, all of the target nucleic acid fragments were the same size as the capture oligonucleotide probe, and all of the target nucleic acid fragments had different compositions (i.e., different numbers of A's, C's, T's and G's), then the number of mass peaks would correspond to the number of different target nucleic acid sequences hybridized to the capture oligonucleotide probe. Since it is possible that target nucleic acid fragments with different sequences have the same composition (i.e., the same number of A's, C's, T's and G's), some different sequences can have the same mass measurements, and hence the number of mass peaks provides the minimum number of different sequences present.

The non-hybridizing end (e.g., the 5′ end or the 3′ end) also can be modified on the basis of its base composition by, for example sequence-specific cleavage such as single base-specific cleavage. For example, if the target nucleic acid fragments used were RNA, and the RNA was first hybridized to the capture probe and then exposed to RNase T1 (which cleaves single-stranded RNA specifically at the 3′ end of G), the non-hybridizing ends of different target probes would vary in length according to the location of the G closest to the hybridizing end of the target nucleic acid. Thus, a method such as base-specific cleavage of the non-hybridizing end can permit control of the non-hybridizing end without requiring the non-hybridizing end to be a pre-defined length prior to the base-specific cleavage.

Base-specific cleavage of the non-hybridizing end can be carried out for any of the four bases that typically occur in nucleic acids. In one embodiment, a sample of target nucleic acids is separated into four separate samples, and each separate sample is hybridized to capture probes on one or four identical chips. After hybridizing to the capture probes, the target nucleic acids of the four chips (or four different locations on one chip) are each subjected to one of four different base-specific cleavage reactions. Finally, the masses of the hybridized target nucleic acids are measured. This four-fold base-specific cleavage also can be done in series, where the four divided samples are serially hybridized to the same chip, treated in one of four base-specific cleavage reactions, and the mass is measured. By measuring the masses of target nucleic acids from four different base-specific cleavage reactions hybridized to the same capture probe, different sequences of the non-hybridizing end that might have the same composition (and therefore the same mass) after one base-specific cleavage, have different compositions (and therefore different masses) after one or more different base-specific cleavages.

Any of a variety of additional combinations of fragmentation, hybridization, and, optionally further fragmentation, can be performed to arrive at a desired complexity, as is recognized by one skilled in the art.

b. Regions of a Fragment

A target nucleic acid fragment can contain at least one, at least two, or at least three regions. For example, a target nucleic acid fragment that contains only one region can be a target nucleic acid in which every nucleotide of the target nucleic acid hybridizes to the capture oligonucleotide probe; a target nucleic acid containing at least two regions can be a target nucleic acid where only a subset of the nucleotides of the target nucleic acid hybridize to the capture oligonucleotide probe (e.g., a target nucleic acid containing two regions can be one where the 3′ end of a target nucleic acid hybridizes to a capture oligonucleotide probe while the 5′ end does not, and vice versa); a target nucleic acid containing at least three regions can be one where the central region of the target nucleic acid, but neither the 5′ end nor the 3′ end, hybridizes to the capture oligonucleotide probe, or can be one where the 5′ end and the 3′ end, but not the central region, hybridizes to the capture oligonucleotide probe; a target nucleic acid having more than three regions can be a target nucleic acid having two or more physically separated regions that hybridize to a capture oligonucleotide probe.

Similarly, capture oligonucleotide probes can have one or more regions. For example, a capture oligonucleotide with two regions can have a first region that hybridizes with a target nucleic acid fragment, and a second region that does not hybridize with at least one target nucleic acid.

c. Partially Single-Stranded Capture Oligonucleotide

In another embodiment, the capture oligonucleotide on the solid-support can be partially double-stranded having a single-stranded overhang. The length of the single-stranded overhang of the capture oligonucleotide is typically 5-6 nucleotides, and also can range from 4 up to 10 nucleotides, or more. When a capture oligonucleotide is partially double-stranded and has for example, a 5 nucleotide single-stranded overhang, a solid-support having 1024 discrete loci can contain capture probes complementary to 5 nucleotides of all possible target nucleic acids. Further, the use of a double-stranded capture oligonucleotide with a single-stranded overhang increases the affinity of the target nucleic acid to the capture oligonucleotide by permitting base-stacking interactions between the capture oligonucleotide probe and one end of the target nucleic acid. By one end of the target nucleic acid base-stacking with the capture oligonucleotide probe, the complexity of one end of the target nucleic acid can be controlled separately from the complexity of the other end.

For example, when a capture probe has a 5 nucleotide single-stranded overhang extending from the 3′ end of one strand, the 5 nucleotides at the 3′ end of the target nucleic acid can hybridize with the capture probe single-stranded overhang. If the capture probe has no degenerate positions, only one 3′ end 5-base sequence of a target nucleotide hybridize to the probe with highest complementarity. If the capture probe has one universal or semi-universal base, only 4 or 2, respectively, 3′ end 5-base sequences of target nucleic acids hybridize to the probe with highest complementarity.

Further in the example, when a capture probe has a 5 nucleotide single-stranded overhang extending from the 3′ end of one strand, target nucleotides can be longer than 5 bases in length; for simplicity in this example, target nucleotides can vary from 5 to 7 bases in length. Thus, nucleotides of 3 different lengths (5 bases, 6 bases and 7 bases) can hybridize to a non-degenerate capture oligonucleotide probe with highest complementarity. Assuming the capture oligonucleotide probe to be non-degenerate, and since each position of the target nucleic acid can have any of four different bases, as many as 21 (4²+4¹+4⁰) different target nucleic acids can hybridize to each non-degenerate capture oligonucleotide probe. If one of the 5 bases in the single-stranded region of the capture probe is a universal base, then as many as 21×4, or 84 target nucleic acids can hybridize to each capture probe. If instead of using a universal base, hybridization conditions were manipulated to permit 1 mismatch at any of the 5 positions where the target nucleotide and the capture probe interact, then as many as 21×4×5 or 420 target nucleic acids can hybridize to each capture probe. Similar calculations can be performed to model the complexity of one region of a target nucleic acid fragment or the complexity of the entire fragment, based on any of a variety of other probes and hybridization stringencies, as is understood by one skilled in the art.

The control of the complexity of the 3′ end separate from the complexity of the 5′ end can be seen in the three above examples. In the examples, the 5′ end sequence is controlled only by the length of the target nucleic acid, and, thus the 5′ end can have as many as 21 different sequences, or more if the length and/or variability of lengths were increased. The 3′ end sequence in this example can be controlled by use of degenerate positions and/or hybridization conditions, such that the complexity of the 3′ end can be varied between 1 and 20 different sequences, or more, if hybridization stringencies were further loosened or additional degenerate positions were included in the capture probe. Further, the complexity of the 3′ end could also be controlled by the number of single-stranded overhanging bases present in the capture probe.

2. Composition of Capture Oligonucleotides

The capture oligonucleotides can have any of a variety of compositions, according to the desired properties of the capture oligonucleotides. For example, the capture oligonucleotide can be single-stranded or contain both single-stranded and double-stranded regions, the capture oligonucleotide can contain universal and/or semi-universal bases, and the capture oligonucleotide can be any of a variety of lengths.

a. Types of Nucleotides

The capture oligonucleotides can contain any of a variety of nucleotides, both naturally occurring and non-naturally occurring. Typically, the capture oligonucleotides contain one or more nucleotides that more favorably hybridize to a first set of nucleotides of the target nucleic acid relative to a second set of nucleotides of the target nucleic acid. For example, a capture oligonucleotide can contain one or more of A, G, C, or T/U.

In some embodiments, the capture oligonucleotides can be partially degenerate and contain one or more degenerate bases. For example, one or more degenerate bases can be “positioned on the 3′ end” of the capture oligonucleotide. Whereas in other embodiments, one or more degenerate bases can be “positioned on the 5′ end” of the capture oligonucleotide. Placement of, for example, one or more universal bases, at one end of the capture oligonucleotide can be useful to enhance hybridization between the capture oligonucleotide and the target nucleic acid without altering the base-specificity of the capture oligonucleotide; such placement can, however, be used to alter the length of the target nucleic acid to which the capture oligonucleotide preferentially binds.

In other embodiments, one or more degenerate bases such as universal and semi-universal bases are located in between specific, non-degenerate bases in a capture oligonucleotide probe. In this manner, a first selected subset of nucleotide positions in the recognition sequence of the capture oligonucleotide probe have increased specificity for particular nucleotides relative to a second subset of nucleotide positions in the recognition sequence of the capture oligonucleotide probe. The distribution of degenerate bases in between non-degenerate bases can take any of a variety of forms, as is recognized by one skilled in the art. Thus, one or more contiguous degenerate bases can be distributed in one or more separate locations in the recognition sequence where the degenerate bases are located in between non-degenerate bases.

i. Universal Bases

The degeneracy of capture oligonucleotides can be achieved using universal bases, which can bind any of the four typically occurring bases of DNA or RNA with similar affinity. Exemplary universal bases for use herein include Inosine, Xanthosine, 3-nitropyrrole (Bergstrom et al., Abstr. Pap. Am. Chem. Soc. 206(2):308 (1993); Nichols et al., Nature 369:492-493; Bergstrom et al., J. Am. Chem. Soc. 117:1201-1209 (1995)), 4-nitroindole (Loakes et al., Nucleic Acids Res., 22:4039-4043 (1994)), 5-nitroindole (Loakes et al. (1994)), 6-nitroindole (Loakes et al. (1994)); nitroimidazole (Bergstrom et al., Nucleic Acids Res. 25:1935-1942 (1997)), 4-nitropyrazole (Bergstrom et al. (1997)), 5-aminoindole (Smith et al., Nucl. Nucl. 17:555-564 (1998)), 4-nitrobenzimidazole (Seela et al., Helv. Chim. Acta 79:488-498 (1996)), 4-aminobenzimidazole (Seela et al., Helv. Chim. Acta 78:833-846 (1995)), phenyl C-ribonucleoside (Millican et al., Nucleic Acids Res. 12:7435-7453 (1984); Matulic-Adamic et al., J. Org. Chem. 61:3909-3911 (1996)), benzimidazole (Loakes et al., Nucl. Nucl. 18:2685-2695 (1999); Papageorgiou et al., Helv. Chim. Acta 70:138-141 (1987)), 5-fluoroindole (Loakes et al. (1999)), indole (Girgis et al., J. Heterocycle Chem. 25:361-366 (1988)); acyclic sugar analogs (Van Aerschot et al., Nucl. Nucl. 14:1053-1056 (1995); Van Aerschot et al., Nucleic Acids Res. 23:4363-4370 (1995); Loakes et al., Nucl. Nucl. 15:1891-1904 (1996)), including derivatives of hypoxanthine, imidazole 4,5-dicarboxamide, 3-nitroimidazole, 5-nitroindazole; aromatic analogs (Guckian et al., J. Am. Chem. Soc. 118:8182-8183 (1996); Guckian et al., J. Am. Chem. Soc. 122:2213-2222 (2000)), including benzene, naphthalene, phenanthrene, pyrene, pyrrole, difluorotoluene; isocarbostyril nucleoside derivatives (Berger et al., Nucleic Acids Res. 28:2911-2914 (2000); Berger et al., Angew. Chem. Int. Ed. Engl., 39:2940-2942 (2000)), including MICS, ICS; hydrogen-bonding analogs, including N8-pyrrolopyridine (Seela et al., Nucleic Acids Res. 28:3224-3232 (2000)); and LNAs such as aryl-β-C-LNA (Babu et al., Nucleosides, Nucleotides & Nucleic Acids 22:1317-1319 (2003); WO 03/020739).

ii. Semi-Universal Bases

A semi-universal base preferentially binds to 2 or 3 of the typically occurring (i.e., A, C, G and T in DNA and A, C, G and U in RNA) nucleotides, but does not bind to all 4 typically occurring nucleotides with the same or similar specificity. For example, a semi-universal base binds to 2 or 3 typically-occurring nucleotides with a greater affinity than it binds to at least one other typically-occurring nucleotide. An exemplary semi-universal base for use herein hybridizes preferentially to either purines A and G, or to pyrimidines C and T. For example, the pyrimidine analog 6H,8H-3,4-dihydropyrimido[4,5-c][1,2]oxazin-7-one hybridizes preferentially with A or G, and the purine analog N6-methoxy-2,6-diaminopurine hybridizes preferentially with C, T or U (see, for example, Bergstrom et al., Nucleic Acids Res. 25:1935-1942 (1997)).

b. Other Characteristics

The sequence, length and composition of a capture oligonucleotide vary according to a variety of factors known to those skilled in the art, including, but not limited to, target nucleic acid molecule length, fragmentation method(s), hybridization conditions, number of different capture oligonucleotides to be used, and desired number of different nucleotide compositions and/or sequences desired to be hybridized to a particular capture oligonucleotide.

In particular embodiments herein, a subset of the capture oligonucleotides can be partially degenerate. For example, embodiments are contemplated herein where at least 10%, at least 20%, at least 30%, at least 40%, at least 50%, at least 60%, at least 70%, at least 80%, at least 90%, at least 95% of the capture oligonucleotides are partially degenerate. In addition, embodiments are contemplated herein where no more than 10%, no more than 20%, no more than 30%, no more than 40%, no more than 50%, no more than 60%, no more than 70%, no more than 80%, no more than 90%, no more than 95% of the capture oligonucleotides are partially degenerate. In other embodiments herein, all of the capture oligonucleotides are partially degenerate. In other embodiments, none of the capture oligonucleotides are partially degenerate.

A partially degenerate capture oligonucleotide can contain a combination of one or more non-degenerate nucleotides (e.g., A, C, G, T for DNA, and A, C, G, U for RNA) and one or more degenerate nucleotides therein (e.g., a universal base or semi-universal base incorporated into the capture oligonucleotide). In another embodiment, a partially degenerate oligonucleotide contains only degenerate nucleotides, where the partially degenerate oligonucleotide still maintains the ability to bind a first set of nucleotide sequences with higher specificity relative to binding a second set of nucleotide sequences. For example, a partially degenerate oligonucleotide can contain only semi-universal bases or a combination of semi-universal bases and universal bases, and the preferential binding of the semi-universal bases confer binding specificity to the partially degenerate oligonucleotide.

The use of partially degenerate capture oligonucleotides permits the binding of more than one specific target nucleic acid sequence to a respective partially degenerate capture oligonucleotide and thereby permits fewer than all theoretical combinations of capture oligonucleotide sequences to be present on the array in order to capture all theoretical combinations of target nucleic acids. The number of degenerate positions used on a particular capture oligonucleotide is selected so that a single capture oligonucleotide is able to preferentially hybridize to two or more different target nucleic acid fragments from the variety of fragments generated during the cleavage step.

As provided elsewhere herein, also contemplated in the use of fewer than all theoretical combinations of capture oligonucleotides, is the lowering or relaxing of the stringency of hybridization conditions to permit mismatch binding, thereby allowing more than one specific target nucleic acid sequence to bind to a respective partially degenerate or non-degenerate capture oligonucleotide, thereby permitting fewer than all theoretical combinations of capture oligonucleotide sequences to be present on the array in order to capture all theoretical combinations of target nucleic acids.

The capture oligonucleotide can be specific for each target nucleic acid fragmentation product or the capture oligonucleotide can be complementary to a common region of two or more different fragments of the target nucleic acid. For example, in a particular hybridization reaction assay, the solid-phase immobilized capture oligonucleotide can hybridize to the fragmentation products of different size that include common subfragment sequences. In addition, a single capture oligonucleotide can be used to capture target-nucleic acid fragments having sequences that differ from each other at the region complementary to the capture oligonucleotide by 1 or more nucleotides, either by using less stringent hybridization conditions and/or by using one or more degenerate nucleotides within the capture oligonucleotide. In other words, the capture nucleotides and stringency conditions can be empirically selected to allow a single capture oligonucleotide sequence to bind to more than one sequence of target nucleic acid fragments. Also, the capture oligonucleotides and stringency conditions can be empirically selected to control the number of different nucleotide fragments with different sequences or nucleotide fragments with different compositions that hybridize to a capture oligonucleotide.

Accordingly, the capture oligonucleotides used herein contain a sequence of nucleotides of sufficient length and sufficient complementarity to semi-specifically hybridize with target nucleic acid fragments prepared herein under the conditions of a contacting or combining step. Before, during or after such hybridization (the hybridization can occur in solution or in solid phase), the capture oligonucleotides are immobilized and arrayed at corresponding discrete, non-overlapping elements on a solid support, such that each element contains a different capture oligonucleotide. A wide variety of materials and methods are known in the art for arraying oligonucleotides at discrete elements of solid supports such as glass, silicon, plastics, nylon membranes, porous material, etc., including contact deposition, e.g., U.S. Pat. Nos. 5,807,522; 5,770,151, etc.; photolithography-based methods, see e.g., U.S. Pat. Nos. 5,861,242; 5,858,659; 5,856,174; 5,856,101; 5,837,832, etc; flow path-based methods, e.g., U.S. Pat. No. 5,384,261; dip-pen nanolithography-based methods, e.g., Piner, et al., Science January 29:661-663 (1999). In a particular embodiment, the capture oligonucleotides are arrayed at corresponding discrete positions (loci) that are generally no more than 20,000, no more than 15,000, no more than 10,000, no more than 7,000, no more than 5,000, no more than 4,000, no more than 3,000, no more than 2500, no more than 2100, no more than 2000, no more than 1500, no more than 1400, no more than 1300, no more than 1200, no more than 1100, no more than 1000, no more than 900, no more than 800, no more than 700, no more than 600, no more than 500, no more than 400, no more than 300, no more than 200, or no more than 100 discrete elements (loci) per each solid-phase array (e.g., a chip).

As set forth herein, the solid-phase array used in the methods provided herein can contain capture oligonucleotides with several degenerate nucleotides therein. This can reduce the total number of oligonucleotides required to capture the information enclosed in the original target nucleic acid sequence. Accordingly, multiple fragments of similar sequence generated during the initial cleavage of the target nucleic acid can hybridize to the same capture oligonucleotide at a respective position. If the multiple species have a different overall nucleotide composition, the mass spectrometric analysis permit their identification by the molecular mass.

In one particular embodiment contemplated herein, the use of universal or semi-universal bases permits hybridization chips with as little as 4096 capture positions, or fewer, to be used for sequencing. Particular applications might require even lower numbers of oligonucleotides. For example, in one embodiment contemplated herein 4096 capture oligonucleotides would allow the creation of all capture oligonucleotides of length 12 for degenerate purine/pyrimidine hybridizing bases (i.e., a 12-base capture oligonucleotide containing 12 semi-universal bases), or capturing oligos with 6 non-degenerate (A,C,G,T) and 6 universal bases, or combinations thereof (e.g., 2 non-degenerate bases, 8 semi-universal bases, and 2 universal bases). The present embodiment does not require each capture oligonucleotide of an array to have the same content of non-degenerate, semi-universal and universal bases in order to create all capture oligonucleotides. For example, some of the capture oligonucleotides can contain only semi-universal bases, while others can contain non-degenerate bases, universal bases and semi-universal bases, and yet others contain only non-degenerate bases and universal bases. The relative amounts of the various types of bases can be determined by one of skill in the art in accordance with the desired level of specificity of the capture oligonucleotides.

In another embodiment, a hybridization structure can have as few as, for example, 1024 capture positions. Such a chip can be used to hybridize multiple samples, for example, four samples that have each been separately treated with conditions that specifically cleave different bases (e.g., sample 1 is treated with A-specific cleavage conditions, sample 2 is treated with C-specific cleavage conditions, sample 3 is treated with G-specific cleavage conditions and sample 4 is treated with T-specific cleavage conditions). In one embodiment, the four samples of the same nucleotide treated with four different cleavage conditions are hybridized to the hybridization structure simultaneously, and the target nucleic acid masses are measured. In another embodiment, the four samples of the same nucleotide treated with four different cleavage conditions are hybridized to the hybridization structure in four separate hybridization steps, where target nucleic acid masses are measured after each of the four separate hybridization steps. In another embodiment, such base-specific cleavage can be selective of single-stranded nucleic acids, so that the portion of the target nucleic acid not bound to the capture oligonucleotide probe is base-specifically cleaved to yield a target nucleic acid longer than the capture oligonucleotide probe to which the target nucleic acid is hybridized (i.e., overhanging the capture nucleotide probe), where the length of the overhang is determined by the location of the nearest specifically cleaved base relative to the hybridized portion of the target nucleic acid.

c. Making the Capture Oligonucleotides

Oligonucleotides can be synthesized separately and then attached to a solid support or synthesis can be carried out in situ on the surface of a solid support. Oligonucleotides can be purchased commercially from a number of companies, including, Integrated DNA Technology (IDT), Fidelity Systems, Proligo, MWG, Operon, MetaBIOn and others.

Oligonucleotides and oligonucleotide derivatives can be synthesized by standard methods known in the art, e.g., by use of an automated DNA synthesizer (such as are commercially available from Biosearch (Novato, Calif.); Applied Biosystems (Foster City, Calif.) and others), combined with solid supports such as controlled pore glass (CPG) or polystyrene and other resins and with chemical methods, such as phosphoramidite method, the H-phosphonate methods or the phosphotriester method. The oligonucleotides also can be synthesized in solution or on soluble supports. For example, phosphorothioate oligonucleotides can be synthesized by the method of Stein et al. (Nucl. Acids Res. 16:3209 (1988)), and methylphosphonate oligonucleotides can be prepared by use of controlled pore glass polymer supports (Sarin et al., Proc. Natl. Acad. Sci. U.S.A. 85:7448-7451 (1988)). Oligonucleotides also can be created using enzymatic methods for amplification, such as, for example PCR or transcription, as disclosed herein and known in the art.

Surface bound capture oligonucleotides are nucleic acids which hybridize to the complementary region on the target nucleic acid fragment. The capture oligonucleotides generally are not substantially involved in any of the reactions that occur to generate the target nucleic acid fragments, such as occur in the chamber of the chip disclosed in related application Ser. Nos. 60/372,711, filed Apr. 11, 2002, 60/457,847, filed Mar. 24, 2003, and Ser. No. 10/412,801, filed Apr. 11, 2003. Preferred oligonucleotides have a number of nucleotides sufficient to allow specific or semi-specific hybridization to the target nucleotide sequence.

Capture oligonucleotides can be any of a variety of lengths, and can include nucleotides that bind to a target nucleic acid nucleotide sequence and nucleotides not intended to bind to a target nucleic acid nucleotide sequence. For example, capture oligonucleotides can contain a portion that hybridizes to a nucleotide sequence that anchors the capture oligonucleotide to a solid support, or a portion that binds a primer sequence of a target nucleic acid fragment (e.g., a transcriptional start site that is not part of the target nucleic acid nucleotide sequence). Capture oligonucleotides also contain nucleotides that can bind to a target nucleic acid nucleotide sequence. The portion of the capture oligonucleotide that binds the target nucleic acid sequence can be any of a variety of lengths, according to factors provided herein and know to those skilled in the art. Typically this portion of the capture oligonucleotide contains 5 up to 30 bases in length. Accordingly, specific lengths of oligonucleotides contemplated for use herein include 4, 5, 6, 7, 8, 9, 10, 11, 12, 13, 14, 15, 16, 17, 18, 19, 20, 21, 22, 23, 24, 25, 26, 27, 28, 29, 30 nucleotides, or more if desired. As set forth herein, oligonucleotides can be made of natural nucleotides, modified nucleotides or nucleotide mimetics (e.g., universal or semi-universal bases) to alter the specificity of hybridization to a complementary sequence or to alter the stability of the formed hybrid.

The specificity of a capture oligonucleotide can be controlled through incorporating degenerate bases or sites into a capture oligonucleotide sequence. Substituting a base within a sequence by inosine can, for example, lead to universal hybridization towards a polymorphic site in target nucleic acid products [see, e.g., Ohtsuka et al. J. Biol. Chem. 260:2605 (1985); Takahashi et al. Proc. Natl. Acad. Sci. U.S.A. 82:1931 (1985)]. The stability of a two-stranded nucleic acid hybrid can be significantly increased by using, for example, RNAs (if directed to a DNA target), locked nucleic acids (LNAs) [Braasch et al. Chemistry & Biology 8:1-7 (2001)], peptide nucleic acids (PNAs) [Armitage et al. Proc. Natl. Acad. Sci. U.S.A. 94:12320-12325 (1997)], or other modified nucleic acid derivatives, completely or partly within the sequence of the capture oligonucleotide or the target nucleic acid sequence. The stability also can be decreased by incorporating one or several abasic sites, non-hybridizing base derivatives or nucleic acid modifications that result in a lower melting temperature, such as phosphorothioates. Various known approaches such as these can be used to modulate the melting temperature for almost any sequence and length to a desired melting temperature.

Oligonucleotide Synthesis

Methods of oligonucleotide synthesis, in solution or on solid supports, are well known in the art [see, e.g., Beaucage et al. Tetrahedron Lett. 22:1859-1862 (1981); Sasaki et al. (1993) Technical Information Bulletin T-1792, Beckman Instrument; Reddy et al., U.S. Pat. No. 5,348,868; Seliger et al. DNA and Cell Biol. 9:691-696 (1990)].

Oligonucleotide Synthesis in situ

Oligonucleotide synthesis in situ on glass and silicon surfaces using light-directed synthesis is well known in the art [see, e.g., McGall et al. J. Am. Chem. Soc. 119:5081-5090 (1997); Wallraffet al. Chemtech 27:22-32 (1997); McGall et al. Proc. Natl. Acad. Sci. U.S.A. 93:13555-13560 (1996); Lipshutz et al. Curr. Opin. Structural Biol. 4:376-380 (1994); and Pease et al. Proc. Natl. Acad. Sci. U.S.A. 91:5022-5026 (1994)].

Oligonucleotides can be attached to a solid support which has been chemically derivatized or a solid support such as polymers or plastic having functional groups. Oligonucleotides can be bound to a solid support by a variety of processes, including photolithography, a covalent bond or passive attachment through noncovalent interactions such as ionic interactions, Van der Waal and hydrogen bonds. Oligonucleotides can be covalently attached to the surface via a 5′ or 3-end modification. Linkers are typically used in order to place the oligonucleotide farther away from the surface. For example, if the oligonucleotide is going to be attached via its 5′-end, then the linker would be on the 5′-end directly proceeding the 5′ modification. Typical linkers used include hexylethyleneglycol (one or more units) and oligodeoxythymidine dTn (with n=5-20).

Various methods can be used for attaching oligonucleotides to surfaces chemically derivatized with reactive functional groups. For example, amino-modified oligos can react with epoxide-activated surfaces to form a covalent bond [see, e.g., Lamture et al. Nuc. Acids Res. 22:2121-2125 (1994)]. Similarly, covalent attachment of amino-modified oligonucleotides can be achieved on carboxylic acid-modified surfaces [Stother et al. J. Am. Chem. Soc. 122:1205-1209 (2000)], isothiocyanate, amine, thio][Penchovsky et al. Nuc. Acids Res. 28:e98 1-6 (2000); Lenigk et al. Langmuir 17:2497-2501 (2001)], isocyanate [Lindroos et al. Nuc. Acids Res. 29:e69 1-7 (2001)] and aldehyde-modified surfaces [Zammatteo et al. Anal. Biochem. 280:143-150 (2000)].

Typically, silicon surfaces can be chemically derivatized followed by immobilization of oligonucleotides as described herein [see also Benters et al. Nuc. Acids Res. 30:e10 1-7 (2002)]. For example, after washing the surfaces, the surface is treated with aminopropyltrimethoxysilane to yield an aminosiloxane layer on the surfaces. The surface is activated with the bifunctional crosslinker 1,4-phenylenediisothiocyanate. One isothiocyanate group of the crosslinker reacts with amino functions on the surface, forming a stable thiourea bond. The second, now surface-bound isothiocyanate group is open for the covalent reaction with other molecules with amino groups. In the following step a dendrimeric polyamine, e.g., Starburst (PAMAM) dendrimer, generation 4 with 64 terminal amino groups, reacts with the activated surface to form a homogeneous interlayer on the solid support with a dense amount of covalently attached amino groups. These functions on the surface are again activated with 1,4-phenylenediisothiocyanate. Unreacted amines are blocked with 4-nitro-phenylene isothiocyanate. Amino-modified oligonucleotides are now covalently cross-linked to the activated dendrimer interlayer through the same type of reaction. In the final step, unreacted isothiocyanates are blocked with a small primary amine, like hexylamine.

Capture oligonucleotides are attached to a solid support in a plurality of discrete known locations or array positions. Each location can contain multiple copies of oligonucleotides having the identical sequence. For example, an array of capture oligonucleotide probes can have multiple copies of oligonucleotides at a particular position, where all oligonucleotides at that particular position have the identical nucleotide sequence, and where the nucleotide sequence of the capture oligonucleotides at that particular position is unique relative to the nucleotide sequence of the capture oligonucleotides at other positions on the array. Thus, an array can be configured such that all oligonucleotides at a particular array position have the identical sequence and all sequences of oligonucleotides at different array positions are unique.

Alternatively, each location can have oligonucleotides having different sequences. This arrangement of oligonucleotides can be used, for example, in multiplex reactions. Oligonucleotides of different sequence at the same location can be mixed together or segregated into groups of like sequence. For example, two, three, four, or more different oligonucleotides can be in the same location. The number of different oligonucleotides utilized is only limited by the ability to resolve the products bound to each different sequence within one location.

Different locations on the solid support typically contain oligonucleotides of different sequence. The oligonucleotides at a location typically occupy an area of 0.0025 mm² to 1.0 mm² with oligonucleotide amounts in the range between 10 amol and 10 pmol. In certain embodiments, a typical format is a solid support, 20×30 mm in size, with 96, 384 or 1536 locations, in an 8×12, 16×24 or 32×48 pattern and spacings that are equivalent to those on a reaction plate (2.25 mm, 1.125 mm or 0.5625 mm center-to-center). Other embodiments can employ up to 4096 positions. In one embodiment, a location is about the diameter of a laser used in one type of mass spectrometric analysis, for example, some locations are no larger than the diameter of the laser. Size of the solid support, the total number of locations and the pattern in which the locations are arranged can conform to design aspects and apparatus used for creating an array on the solid support, for liquid handling and/or for analysis. For example, the spacing and spot size can be such that it is dictated by the accuracy and/or the drop size of an instrument that creates the array. The number of locations of oligonucleotides placed in a row or column on a solid support can be such that the laser of a MALDI-TOF mass spectrometer does not encompass more than one location at the same time.

Groups of capture oligonucleotides can be positioned on the solid support surface in any arrangement. For example, oligonucleotides can be placed in individual wells or chambers made in the solid support. The number of wells present on the solid support can vary depending on the size of the solid support, with a 96 or 384 format often used, as well as formats up to 4096 or more readily available. Typically, the wells or chambers remain separate and maintain their integrity. In one example, oligonucleotides can be placed on the solid support at discrete known locations in rows or columns that share a common overlying reagent channel. In another example, oligonucleotides also can be arranged atop a totally flat surface in such discrete known locations and in any arrangement. The location also can be subdivided in smaller areas with individual oligonucleotides or mixes of oligonucleotides. Channels or wells for reagents can be created with masks made of the same or a different material placed on top of the solid support. Furthermore, wells and channels on the solid support can be designed in a way that they localize or even separate and sort beads, for example according to their size. In this design, the beads are carriers of the oligonucleotides used for the capturing of reaction product nucleic-acid-fragments and derivatives.

F. Solid Supports and Arrays

The methods provided herein can utilize the capture onto a solid-support of fragments of the target nucleic acid that is to be sequenced. Solid supports can be formed from any materials that are used as affinity matrices or supports for chemical and biological molecule syntheses and analyses, such as, but are not limited to: polystyrene, polycarbonate, polypropylene, nylon, glass, metal, magnetic beads, latex, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications. The solid support herein can be particulate or can be in the form of a continuous surface, such as a coated pin tool, a microtiter dish or well, a glass slide, a metal, plastic or silicon chip, a nitrocellulose sheet, nylon mesh, a porous three-dimensional structure such as a porous three-dimensional gel, or other such materials. When particulate, typically the particles have at least one dimension in the 5-10 mm range or smaller. Such particles, referred collectively herein as “beads”, are often, but not necessarily, spherical. Such reference, however, does not constrain the geometry of the solid support, which can be any shape, including random shapes, needles, fibers, and elongated. Roughly spherical “beads”, particularly microspheres that can be used in the liquid phase, also are contemplated. The “beads” can include additional components, such as magnetic or paramagnetic particles (see, e.g., Dynabeads7 (Dynal, Oslo, Norway)) for separation using magnets, as long as the additional components do not interfere with the methods and analyses herein.

For example, in a particular embodiment a hybridization chip set forth in related Unites States application Ser. Nos. 60/372,711, filed Apr. 11, 2002, 60/457,847, filed Mar. 24, 2003, and Ser. No. 10/412,801, filed Apr. 11, 2003, is used as the solid support for the array of capture oligonucleotides, e.g., target-nucleic acid fragments are captured by the capture oligonucleotide on the surface of a solid-phase solid support on the interior bottom surface of a chamber, over which the target nucleic acid fragment generating reaction(s) are performed. In a particular embodiment, the fragmentation reaction(s) is performed in a chamber that contains, or the bottom of the chamber is, a solid support that is capable of specifically hybridizing with the target nucleic acid fragmentation product in such a way as to retain it attached to the solid support during processes used to remove or wash other molecules from the chamber. The interaction can be between the target nucleic acid fragmentation product and a capture oligonucleotide that has been immobilized on the solid support e.g., a derivatized or functionalized solid support. Any type of solid support can be used that achieves the specific capture of the target nucleic acid fragmentation product(s).

For example, the solid support can be a flat two dimensional surface or three-dimensional surface, or can be beads. In the case of a flat solid support, the chamber can be formed by walls that extend out from the solid support surface, e.g., as provided by a “mask” as described in an embodiment of an apparatus provided herein, or that are made by etching wells or pillars or channels into the solid support surface in order to create discrete and isolated chambers. Possible materials of which solid supports can be made include, but are not limited to, silicon, silicon with a top oxide layer, glass, metal such as platinum or gold, polymers such as polyacrylamide, and plastic. In a particular embodiment the solid support is a silicon chip or wafer.

Flat solid supports can also be modified to contain a thermoconductive material to facilitate temperature regulation of the reaction mixture in the chamber. In a particular embodiment, the solid support is a flat silicon chip coated with a metal material. Exemplary solid supports are described herein and can be used in conjunction with devices and methods described and provided herein.

As set forth above, the capture oligonucleotides are arrayed at corresponding discrete elements at a number of positions (loci) that is generally no more than 20,000, no more than 15,000, no more than 10,000, no more than 7,000, no more than 5,000, no more than 4,000, no more than 3,000, no more than 2500, no more than 2100, no more than 2000, no more than 1500, no more than 1400, no more than 1300, no more than 1200, no more than 1100, no more than 1000, no more than 900, no more than 800, no more than 700, no more than 600, no more than 500, no more than 400, no more than 300, no more than 200, no more than 100 discrete elements per each solid-support (e.g., a chip). In further embodiments, the array contains 4096 or fewer, 1536 or fewer, 384 or fewer, 96 or fewer, 64 or fewer discrete positions having capture oligonucleotides. In a particular embodiment, the array of capture oligonucleotides contains 4096 capture oligonucleotides. In one embodiment where the array contains 4096 oligonucleotides, the capture oligonucleotides can be 12 bases in length. In other embodiments using an array of 4096 oligonucleotides, capture oligonucleotides can be 30 bases in length, 25 bases in length, 20 bases in length, 15 bases in length, 10 bases in length, 9 bases in length, 8 bases in length, 7 bases in length, and 6 bases in length.

In particular embodiments, all of the capture oligonucleotides on the solid supports are fully or partially degenerate, e.g., they contain at least one universal or semi-universal base therein. In other embodiments, the solid supports can contain combinations of fully degenerate, partially degenerate and/or non-degenerate capture oligonucleotides therein. A non-degenerate capture oligonucleotide is one that does not contain any degenerate bases (universal or semi-universal bases) therein.

The array of capture oligonucleotides can be designed in a variety of manners according to the desired properties of the capture oligonucleotides. The capture oligonucleotides that make up the array can be varied in length, sequence, composition, or presence/absence of a double-stranded portion, and combinations thereof. For example, an array can be designed to have all single-stranded capture oligonucleotides 12 bases in length and include 6 universal bases per capture oligonucleotide. Alternatively, the array can be designed to contain 50% single-stranded and 50% partially double-stranded oligonucleotides of a variety of different lengths and/or a variety of different compositions (e.g., different numbers of universal bases and/or semi-universal bases), or both. For example, an array can be designed to contain capture oligonucleotides that vary in length from 6 to 18 bases in length, and can, in addition or as an alternative, be designed to contain capture oligonucleotides that contain between 6 and 12 universal or semi-universal bases.

Typically, an array of capture oligonucleotide probes contain capture oligonucleotide probes that are 4 or more nucleotides in length, 5 or more nucleotides in length, 6 or more nucleotides in length, 7 or more nucleotides in length, 8 or more nucleotides in length, 10 or more nucleotides in length, 12 or more nucleotides in length, or 15 or more nucleotides in length. Additionally, a typical array of capture oligonucleotide probes contains capture oligonucleotide probes that are no more than 50 bases in length, no more than 40 bases in length, no more than 35 bases in length, no more than 30 bases in length, no more than 25 bases in length, no more than 20 bases in length, no more than 18 bases in length, no more than 16 bases in length, no more than 14 bases in length, no more than 12 bases in length, no more than 10 bases in length, or no more than 8 bases in length. Further, a capture oligonucleotide probe can have one or more additional degenerate bases at the 3′ end, 5′ end or both the 3′ end and the 5′ end.

The size, composition, and presence/absence of double-stranded portions of the capture oligonucleotides in the designed array can be selected with any of a variety of desired purposes. In one embodiment, the array can be designed to contain arrays that each hybridize with about the same number of different sequences of target nucleic acids under the same stringency conditions. For example, the array can be designed to contain capture oligonucleotides that each hybridize with a perfectly complementary sequence(s) under the same hybridization conditions (e.g., have the same melting temperatures). This can be accomplished, for example, by designing primers with the same (A+T)/(C+G) ratios, by making C/G-rich capture oligonucleotides shorter than A/T-rich capture oligonucleotides, varying the length of capture oligonucleotides, including universal or semi-universal bases, or including capture oligonucleotides with double-stranded regions. In another example, the array can be designed with capture oligonucleotides having different melting temperatures, but hybridizing to the same number of different target nucleic acids under particular conditions. For example, a capture oligonucleotide with a higher melting temperature can be shorter in length or contain more universal or semi-universal bases relative to a capture oligonucleotide with a lower melting temperature. As such, under some hybridization conditions, the capture oligonucleotides can hybridize to the about same number of different target nucleic acid sequences. For example, the portion of a first capture oligonucleotide that hybridizes with a target nucleic acid fragment can contain only a few nucleotides, but the nucleotides can be mainly G's and C's, resulting in a variety of different target nucleic acid fragments bound because the target nucleic acid sequences in the portion of the target nucleic acid that does not hybridize to the first capture oligonucleotide is not constrained; for a second capture oligonucleotide the portion that hybridizes with a target nucleic acid fragment can contain more nucleotides, but the nucleotides can include universal or semi-universal bases that hybridize more weakly than G's and C's, resulting in a variety of different target nucleic acid fragments bound because the target nucleic acid sequences that bind to the capture oligonucleotide can vary according to the number of degenerate bases in the capture oligonucleotide; as a result, the total number of different target nucleic acid sequences that hybridize to the first and second capture oligonucleotides at any particular hybridization conditions can be about the same.

Alternatively, the size and compositions of the capture oligonucleotides in the designed array also can be selected such that different capture oligonucleotides hybridize to varying numbers of different target nucleic acids under selected hybridization conditions. For example, a first capture oligonucleotide can be designed to hybridize with 20 different target nucleic acids under the same conditions that result in a second capture oligonucleotide hybridizing with 10 different target nucleic acids. For example, a first capture oligonucleotide can contain 6 non-degenerate bases and 6 universal bases, while a second capture oligonucleotide can contain the same 6 non-degenerate bases as the first capture oligonucleotide, plus two additional non-degenerate bases; as a result, only a subset of the target nucleic acids that bind the first capture oligonucleotide also bind to the second capture oligonucleotide.

The size, composition, and nucleotide sequence of the capture oligonucleotides in the designed array also can be selected in order to meet one or more of the following criteria: target particular types of sequences such as, for example, SNPs or microsatellites; target random or unknown sequences; control the complexity of the target nucleic acids at different regions (e.g., by having some of the capture oligonucleotides double-stranded in order to control the complexity of the end sequence portions of some of the target nucleic acids); and increase or decrease the number of overlapping fragments that hybridize to a particular capture oligonucleotide (e.g., decrease by using a large percentage of universal or semi-universal bases, or increase by using shorter, specific sequences with no double-stranded region and no universal bases at any position except, optionally, at one or both ends).

G. Specific or Non-Specific Hybridization

The methods provided herein typically include steps of hybridizing two or more nucleic acid molecules. In the present methods, a capture oligonucleotide can hybridize with one or more target nucleic acid molecules or fragments thereof to form a “capture oligonucleotide:target fragment complex” or a “capture oligonucleotide:target nucleic acid complex”. Such complexes are often double-stranded complexes (i.e., duplexes), but also can be triple-stranded complexes.

The extent and specificity of hybridization varies with reaction conditions, particularly with respect to temperature and salt concentrations. Hybridization reaction conditions typically are referred to in terms of degree of stringency, e.g., low, medium and high stringency, which are achieved under differing temperatures and salt concentrations known to those of skill in the art and exemplified herein. Thus, in one embodiment for example, to reduce the amount of imperfect matches between hybridizing nucleic acids, higher stringency conditions can be employed, e.g., higher temperatures and/or lower salt concentrations. Conversely, to increase the amount of imperfect matches permitted between hybridizing nucleic acids, lower stringency conditions can be employed, e.g., lower temperatures and/or higher salt concentrations.

In particular embodiments, the capture oligonucleotides used to hybridize to target nucleic acid fragments do not hybridize with complete base-specificity, and therefore do not eliminate mismatched hybridization or degeneracy in hybridization. This permits the hybridization stringency to be lowered, such that not all theoretical combinations of nucleotide capture sequences need to be represented on the chip array. As set forth herein, the degeneracy of the capture oligonucleotides and the hybridization stringency conditions can be varied empirically to permit as few as 4096, or fewer, capture oligonucleotides on the solid-support. The composition and sequence of a mismatched fragment can be identified by acquiring the molecular mass in a subsequent mass spectrometric analysis.

The amount of mismatched hybridization advantageously utilized in the methods provided herein is significantly more than the undesired amount of mismatch hybridization that occurs in typical SBH methods under conditions that attempt to eliminate such mismatch hybridization. For example, a capture oligonucleotide used in accordance with the methods provided herein can have two or more target nucleic acid fragments hybridized thereto. In some instances, two or more target nucleic acid fragments can be hybridized with perfect complementarity to the capture oligonucleotide; examples of such instances are two or more target nucleic acid fragments hybridized to a capture oligonucleotide containing two or more degenerate nucleotides, or two or more target nucleic acid fragments that are longer than the capture oligonucleotide and vary in sequence according to the portion of the fragments not hybridized to the capture oligonucleotide. In other instances, hybridization conditions can be selected to have reduced stringency such that two or more target nucleic acid fragments can hybridize to a capture oligonucleotide; in such instances, it can be desirable for one or more target nucleic acid fragments to hybridize to a capture oligonucleotide with less than perfect complementarity. Exemplary resultant mixtures of target nucleic acid fragments hybridized to a capture oligonucleotide include mixtures of target nucleic acid fragment where no particular target nucleic acid fragment is present in the mixture of target nucleic acid fragments hybridized to a capture oligonucleotide as more than 95%, 90%, 85%, 80%, 75%, 70%, 65%, 60%, 55%, 50%, 45%, 40%, 35%, 30%, or 25% of the target nucleic acid fragments in the mixture. In another example, resultant mixtures include mixtures of target nucleic acid fragments where at least two, at least three, at least four, or at least five target nucleic acid fragments are present in an amount more than 5%, 10%, 15%, or 20%, of the target nucleic acid molecule hybridized to the capture oligonucleotide. In another example, no target nucleic acid fragment is present in an amount that is more than 2-fold, more than 3-fold, more than 4-fold, or more than 5-fold the amount of at least one other target nucleic acid fragments in the mixture of target nucleic acid fragments hybridized to a capture oligonucleotide (i.e., relative to the most abundant target nucleic acid fragment, there is present at least one other fragment in an amount that is at least 50%, 33%, 25% or 20% of the amount of most abundant fragment).

In particular embodiments, the capture oligonucleotides are designed such that each chip position (typically having multiple copies of the same capture oligonucleotide) bind to two or more of the target nucleic acids fragments. For example, conditions are contemplated herein such that 2 up to 500, 2 up to 400, 2 up to 300, 2 up to 250, 2 up to 200, 2 up to 150, 2 up to 100, 2 up to 75, 2 up to 50, 2 up to 40, 2 up to 30, 2 up to 25, 2 up to 20, 2 up to 15, 2 up to 10, or 2 up to 5 different target nucleic acid fragments bind to a single species of capture oligonucleotide. In such instances, different target nucleic acid fragments includes the binding of fragments that are sub-fragments of other fragments (e.g., creating ladders of fragments), as well as the binding of fragments having the same or different lengths and having similar hybridization properties for the particular chip position and capture oligonucleotide, but having different nucleotide compositions.

In some embodiments, methods that include two or more different hybridization reactions (e.g., an array with two or more discrete loci with which target nucleic acid fragments are contacted) do not require that all of the two or more hybridization reactions (e.g., array positions) result in capture oligonucleotides having two or more target nucleic acid fragments hybridized thereto. In some instances, some reactions (e.g., array positions) can contain no target nucleic acid fragments hybridized thereto. In other instances, some reactions (e.g., array positions) can contain only one target nucleic acid fragment hybridized thereto. Typically, at least 50%, at least 55%, at least 60%, at least 65%, at least 70%, at least 75%, at least 80%, at least 85%, at least 90%, at least 95%, at least 96%, at least 97%, at least 98%, or at least 99%, of all reactions result in two or more oligonucleotides hybridized to capture oligonucleotides, where the relative amounts of the two or more capture oligonucleotides are present at levels as provided herein.

To increase the hybridization efficiency, the capture oligonucleotides can be elongated by universal bases. For example, a capture oligonucleotide can contain two regions: a first region containing only universal bases, and a second region containing at least one typically occurring or semi-universal base. The second region contains bases that are used for specifically or semi-specifically hybridizing with target nucleic acids, while the universal bases of the first region serve to stabilize the hybridization between a capture oligonucleotide and a target nucleic acid.

In addition, because multiple target nucleic acids can hybridize with a single capture oligonucleotide, the capture oligonucleotide can incorporate degenerate bases in the sequence recognition portion of the capture oligonucleotide, resulting in a degenerate capture oligonucleotide. If the total number of chip array positions is to be kept low, the length and/or specificity of the sequence recognition portion of a degenerate capture oligonucleotides is limited.

In one embodiment, capture oligonucleotides of a targeted length of 12 nucleotides would be placed in 4096 positions. Addition of further universal bases to one end of the capture oligonucleotide would therefore increase the stability of the hybridization complex significantly and increase the overall efficiency, without modifying the sequence specificity of the capture oligonucleotide. Depending on further modifications, in one embodiment, these additional universal nucleotides could be placed towards the 3′ end of the capture oligonucleotide. In another embodiment, these additional universal nucleotides could be placed towards the 5′ end of the capture oligonucleotide. In another embodiment, the additional universal nucleotides can be placed at both ends of a capture oligonucleotide.

Further modifications to the hybridized fragments are possible to increase the information content and the flexibility and robustness of the system, or to reduce the compositional complexity of the system. For example, treatment of the capture oligonucleotide:target fragment duplex on the solid-phase array with single-strand specific RNases or DNases (“trimming reaction”) reduce the overall length of hybridized fragments to a more uniform length. Use of trimming can influence the selection of initial fragmentation conditions. For example, the limitations imposed during an initial random fragmentation method can be relaxed and the upper limit for fragment sizes can be increased. Hybridized fragments of size 35 bases or more can be shortened towards the length of the capture oligo and/or to a size readily detected by MALDI-MS. Relaxation of fragmentation parameters is contemplated herein to improve the flexibility of the system for various sequences. Additionally, base-specific RNases or DNases (“base-specific trimming”) can be used, which do not necessarily shorten the hybridized fragment to the exact length of the capture oligo, but can shorten the target nucleic acid fragment to the targeted base nearest to the capture oligo. Such base-specific cleavage can target any of the 4 bases in the nucleotide, and can thus result in the same hybridized fragment being modified to one of four different fragments according to the particular base-specific cleavage reaction.

The step of hybridizing the capture oligonucleotide with target fragments involves selectively controlling the relative affinity of the capture oligonucleotides for the corresponding target nucleic acid fragments sufficiently to provide the desired level hybridization of the capture oligonucleotide to the corresponding target nucleic acid fragments(s), while eliminating the relative affinity of the capture oligonucleotide to non-corresponding target nucleic acid fragments. As set forth herein, in one embodiment, stringency conditions are selected to permit one or more mismatches in the capture oligonucleotide:target fragment duplex. Thus, the target fragments corresponding to a particular capture oligonucleotide not only include fragments containing the exact complementary sequence therein, but also can include target nucleic acid fragments having at least one or more nucleotide mismatches therein. In aggregate, the relative affinity of a capture oligonucleotide for mismatched target nucleic acids is generally measured as the ratio of the capture oligonucleotides binding to one or more mismatched target nucleic acid fragments (e.g., having at least a single base mismatch between the capture oligonucleotide and the target nucleic acid) relative to the capture oligonucleotides binding to perfectly complementary target nucleic acid fragments. An increase in the ratio refers to an increase in the binding of capture oligonucleotides to mismatched target nucleic acid fragments relative to the binding of capture oligonucleotides to perfectly matched oligonucleotides. The ratio used herein can be varied accordingly, and generally is at least about 0.5 fold (i.e., the capture oligonucleotide probe binds 1 mismatched target nucleic acid for every two perfectly complementary target nucleic acid fragments bound), at least about 1 fold, at least about 1.5 fold, at least about 2 fold, at least about 3 fold, at least about 5 fold, at least about 7 fold, at least about 10 fold, at least about 15 fold, or at least about 20 fold. One skilled in the art can select the ratio based on a variety of factors, including the length of the target nucleic acid being studied, the length and numbers of different target nucleic acid fragments, the ability to resolve measured mass peaks, and the ability to use the measured mass peaks in determining the nucleic acid sequence of the target nucleic acid.

A variety of methods or assay conditions can be used to modulate the relative affinity of each capture oligonucleotide for the corresponding target nucleic acid (e.g., a target nucleic acid bound by a capture oligo with specific or semi-specific affinity). In one particular embodiment, the relative affinity of each capture oligonucleotide for the corresponding target nucleic acid is increased at least in part by a method comprising the step of including in the hybridization step a reagent which normalizes the melting temperatures of the hybrids formed with the assay probes, in particular, normalizing the melting temperatures of the hybrids formed between the target nucleic acids and capture oligonucleotides sufficient to provide the desired discrimination between the corresponding target nucleic acid and other non-corresponding target nucleic acids. A wide variety of suitable normalizing reagents, including detergents (e.g., sodium dodecyl sulfate, Tween), denaturants (e.g., guanidine, quaternary ammonium salts), polycations (e.g., polylysine, spermine), minor groove binders (e.g., distamycin, CC-1065, see Kutyavin, et al., 1998, U.S. Pat. No. 5,801,155), etc. and their use are described herein and/or otherwise known in the art. Effective concentrations and suitable assay conditions are readily determined empirically (see, e.g., Examples, below).

In a particular embodiment, the denaturant is a quaternary ammonium salt such as tetramethyl ammonium chloride, tetraethyl ammonium chloride, tetramethyl ammonium fluoride or tetraethyl ammonium fluoride. Normalization of melting temperatures can be confirmed by any convenient means, such as a reduction in the coefficient of variance (CV) or standard deviation of the melting temperatures. For example, melting temperatures can be normalized by a reduction of the CV or standard deviation of at least 20%, at least 40%, at least 60%, or at least 80%. An increase in the ratio between the signal of a perfect match and for a single base mismatch indicates that a less stringent CV may be required. Stringency conditions that produce the following exemplary ratios of matches to mismatches are contemplated for use herein and include ratios of 2:1 match to mismatch, 3:1, 4:1, 5:1, 6:1, 7:1, 8:1, 9:1, 10:1, 15:1, 20:1 match to mismatch, and so on. For an exemplary ratio of 5:1 match to mismatch, CVs of 20% or lower are desired, as well as CVs of 10% or lower; while for a ratio of 50:1 match to mismatch, CVs of 50% or lower are desired.

Control of the number of target nucleic acid sequences that hybridize to a particular capture oligonucleotide probe can be accomplished by either use of universal or semi-universal bases, or by modifying hybridization conditions, or both. Use of universal base composition and hybridization represent two separate and independent methods for controlling the number of target nucleic acid sequences that hybridize to a particular oligonucleotide probe. One skilled in the art can choose either to use universal or semi-universal bases, or to modify hybridization conditions, or both, based on the desired complexity of target nucleic acid fragments hybridized to capture oligonucleotides.

Universal bases can be used to control the theoretical number of different target nucleic acid sequences that can base pair to the capture oligonucleotide with the same or similar affinity, and also can be useful for determining the position on the portion of the target nucleic acid that base-pairs with the capture oligonucleotide without sequence specificity. For example, use of two universal bases in a capture probe permits up to 16 different target nucleic acid sequences to base pair with the capture probe with similar affinity, and the location on the capture oligonucleotide of the non-universal bases can be known. Thus, the number of target nucleic acid sequences that base-pair with the capture oligonucleotide can be controlled, and the nucleotide positions on the target nucleic acid where the nucleotide sequence is variable can be known.

Manipulation of hybridization conditions permits the user to readily modify the hybridization conditions in order to achieve a desired number of different target nucleic acid sequences that actually hybridize to a capture oligonucleotide probe. For example, the number of different target nucleic acid sequences that hybridize to a capture oligonucleotide probe under particular hybridization conditions can be experimentally determined. After such an experimental determination, if desired, the hybridization conditions can be relaxed to permit more hybridization of various different target nucleic acid fragments to a capture oligonucleotide probe; or the hybridization conditions can be made more stringent in order to reduce the number of different target nucleic acid fragments that hybridize to a capture oligonucleotide. The hybridization conditions can be changed several times in order to select hybridization conditions that yield the desired number of different target nucleic acid fragments that hybridize to a capture oligonucleotide probe.

Stringency conditions for removing the non-specific binding of capture oligonucleotides to target nucleic acid fragments, and conditions that are substantially equivalent to either high, medium, or low stringency include the following:
1) high stringency: 0.1×SSPE, 0.1% SDS, 65 EC
2) medium stringency: 0.2×SSPE, 0.1% SDS, 50 EC
3) low stringency: 1.0×SSPE, 0.1% SDS, 50 EC;
   where SSPE generally contains about 150 mM NaCl, 10 mM NaH₂PO₄, 1 mM EDTA, pH 7.0, or components equivalent thereto.

It is understood that equivalent stringencies can be achieved using alternative buffers, salts and temperatures. In particular embodiments, in order to allow the capture of more than 1 specific target nucleic acid fragment sequence on one or more of the capture oligonucleotides, the hybridization stringency conditions could be relaxed to medium or low stringency for capture oligonucleotides having few to no degenerate nucleotides therein. Likewise, when several degenerate oligonucleotides are contained within the capture oligos, the hybridization conditions can be made more stringent, for example, hybridization conditions can be high stringency conditions. The conditions can be empirically selected such that mismatch hybridization is not completely eliminated, but at the same time, only a subset of fragmented target nucleic acids can bind to a particular capture oligo; stringency conditions can be modified to attain the desired size of the subset of target nucleic acid fragments that bind.

In one embodiment, the hybridization conditions can be changed from the initial hybridization conditions. The change can be either lowering or raising the stringency of hybridization conditions. For example, hybridization can be carried out initially under low stringency hybridization conditions; then, later, the hybridization conditions can be raised to medium or high stringency hybridization conditions. In and alternative example, hybridization conditions can be carried out initially under high stringency hybridization conditions; then, later, the hybridization conditions can be lowered to medium or low stringency hybridization conditions.

In one embodiment, hybridization conditions can be changed to modify the number of target nucleic acids that hybridize to a capture oligonucleotide probe. For example, stringency of hybridization conditions can be raised to decrease the number of target nucleic acids that hybridize to a capture oligonucleotide probe. Alternatively, stringency of hybridization conditions can be lowered to increase the number of target nucleic acids that hybridize to a capture oligonucleotide probe. Thus, as contemplated herein, hybridization conditions can be modified to achieve a desired number of target nucleic acids that hybridize to a capture oligonucleotide probe.

The number of target nucleic acids hybridized with capture oligonucleotide probes can be determined by any method known in the art for measuring nucleic acids bound to an oligonucleotide array, including: optical measurements such as fluorescence or absorbance, which can be carried out, for example, on an oligonucleotide array such as an oligonucleotide chip; detection of a scattering, radioactive, chemiluminescent, calorimetric, or magnetic label; mass spectrometry of one or more array positions; or other methods known in the art such as those disclosed in U.S. Pat. No. 6,045,996.

One or more measurements of the number of target nucleic acids hybridized to one or more capture oligonucleotide probes can be used to compare the actual number of target nucleic acids hybridized to the capture oligonucleotide probes to the desired number of target nucleic acids hybridized to the capture oligonucleotide probes. Upon measurement of the number of target nucleic acids hybridized to the one or more capture oligonucleotide probes, hybridization conditions can be modified to increase or decrease the number of target nucleic acids hybridized to the capture oligonucleotide probes, whichever is desired. Such a process can be carried out iteratively until the desired number of target nucleic acids hybridized to the one or more capture oligonucleotide probes is achieved.

H. Trimming

In some embodiments, the single-stranded overhanging portion of the capture oligonucleotide:target fragment duplex can be trimmed down in size to facilitate the subsequent mass spectrometric analysis of the duplex and to reduce compositional complexity. Trimming can be performed, for example, when the average size of the target nucleic acid fragments is relatively large, or when there is a large range of different sizes of target nucleic acid fragments. Trimming can be performed to reduce the size of target nucleic acid fragments to be measured by mass spectrometry. Trimming also can be performed to reduce the range of different sizes of target nucleic acid fragments to be measured by mass spectrometry, and/or to reduce the mass of fragments to be measured by mass spectrometry.

Trimming methods can be performed by any of a variety of known methods. For example, trimming can be performed by further treating the array of captured fragments with an enzyme or chemical to remove unhybridized nucleotides. An enzyme can, for example, be any exonuclease known in the art or a “single-strand specific RNase or DNase” or a “base-specific RNase or DNase”, or a sequence-specific nuclease. In another example, an endonuclease, such as a single-strand specific endonuclease can be used to trim unhybridized nucleotides; in such trimming reactions, not all unhybridized nucleotides are necessarily removed. A single-strand specific endonuclease can be sequence specific, or sequence unspecific. For example, an enzyme can be a base-specific RNase or DNase, and hybridized fragments larger than the capture oligonucleotide can have either the 3′ or 5′ end, or both, trimmed as a function of the presence of one or more of A, C, G or T/U.

I. Information Relating to the Target Nucleic Acid Fragments

The methods for reconstructing the nucleic acid sequence of the target nucleic acid, and other methods disclosed herein, including identifying a portion of a target nucleic acid, can utilize a variety of information relating to target nucleic acids and target nucleic acid fragments provided in the methods herein to reconstruct the sequence or identify a portion of the target nucleic acid. Such information includes mass measurement, mass peak characteristics, the sequence of the capture oligonucleotide to which the target nucleic acid hybridized, hybridization conditions, and the fragmentation method(s) used.

1. Molecular Mass

As set forth herein, the step for reconstructing the nucleic acid sequence of the target nucleic acid, and other methods disclosed herein, including identifying a portion of a target nucleic acid, can utilize determining the molecular mass of target nucleic acid fragments hybridized to a capture nucleic acid, or capture oligonucleotide:target fragment duplexes to thereby determine the mass of target nucleic acid fragments.

a. Mass Spectrometric Analysis

Mass spectrometric analysis can be used in the determination of the mass of particular molecules. Such formats include, but are not limited to, Matrix-Assisted Laser Desorption/Ionization, Time-of-Flight (MALDI-TOF), Electrospray ionization (ESi), IR-MALDI (see, e.g., published International PCT application No. 99/57318 and U.S. Pat. No. 5,118,937), Orthogonal-TOF (O-TOF), Axial-TOF (A-TOF), Ion Cyclotron Resonance (ICR), Fourier Transform, Linear/Reflectron (RETOF), and combinations thereof. See also, Aebersold and Mann, Mar. 13, 2003, Nature, 422:198-207 (e.g., at FIG. 2) for a review of exemplary methods for mass spectrometry suitable for use in the methods provided herein, which is incorporated herein in its entirety by reference. MALDI methods typically include UV-MALDI or IR-MALDI. Nucleic acids can be analyzed by detection methods and protocols that rely on mass spectrometry (see, e.g., U.S. Pat. Nos. 5,605,798, 6,043,031, 6,197,498, 6,428,955, 6,268,131, and International Patent Application No. WO 96/29431, International PCT Application No. WO 98/20019). These methods can be automated (see, e.g., U.S. Publication 2002 0009394, which describes an automated process line). Medium resolution instrumentation, including but not exclusively curved field reflectron or delayed extraction time-of-flight MS instruments, also can result in improved DNA detection for sequencing or diagnostics. Either of these are capable of detecting a 9 Da (Δm(A−T)) shift in ≧30-mer strands.

When analyses are performed using mass spectrometry, such as MALDI, nanoliter volumes of sample can be loaded on chips. Use of such volumes can permit quantitative or semi-quantitative mass spectrometric results. For example, the area under the peaks in the resulting mass spectra are proportional to the relative concentrations of the components of the sample. Methods for preparing and using such chips are known in the art, as exemplified in U.S. Pat. No. 6,024,925, U.S. Publication 2001 0008615, and PCT Application No. PCT/US97/20195 (WO 98/20020); methods for preparing and using such chips also are provided in co-pending U.S. application Ser. Nos. 08/786,988, 09/364,774, and 09/297,575. Chips and kits for performing these analyses are commercially available from SEQUENOM under the trademark MassARRAY7. MassARRAY7 systems contain a miniaturized array such as a SpectroCHIP7 array useful for MALDI-TOF (Matrix-Assisted Laser Desorption Ionization-Time of Flight) mass spectrometry to deliver results rapidly. It accurately distinguishes single base changes in the size of DNA fragments relating to genetic variants without tags.

i. Characteristics of Nucleic Acid Molecules Measured

In one embodiment, the mass of all nucleic acid molecule fragments formed in the step of fragmentation is measured. The measured mass of a target nucleic acid molecule fragment or fragment of an amplification product also can be referred to as a “sample” measured mass, in contrast to a “reference” mass which arises from a reference nucleic acid fragment.

In another embodiment, the length of nucleic acid molecule fragments whose mass is measured using mass spectroscopy is no more than 75 nucleotides in length, no more than 60 nucleotides in length, no more than 50 nucleotides in length, no more than 40 nucleotides in length, no more than 35 nucleotides in length, no more than 30 nucleotides in length, no more than 27 nucleotides in length, no more than 25 nucleotides in length, no more than 23 nucleotides in length, no more than 22 nucleotides in length, no more than 21 nucleotides in length, no more than 20 nucleotides in length, no more than 19 nucleotides in length, or no more than 18 nucleotides in length.

In another embodiment, the length of the nucleic acid molecule fragments whose mass is measured using mass spectroscopy is no less than 3 nucleotides in length, no less than 4 nucleotides in length, no less than 5 nucleotides in length, no less than 6 nucleotides in length, no less than 7 nucleotides in length, no less than 8 nucleotides in length, no less than 9 nucleotides in length, no less than 10 nucleotides in length, no less than 12 nucleotides in length, no less than 15 nucleotides in length, no less than 18 nucleotides in length, no less than 20 nucleotides in length, no less than 25 nucleotides in length, no less than 30 nucleotides in length, or no less than 35 nucleotides in length.

In one embodiment, the nucleic acid molecule fragment whose mass is measured is RNA. In another embodiment the target nucleic acid molecule fragment whose mass is measured is DNA. In yet another embodiment, the target nucleic acid molecule fragment whose mass is measured contains one modified or atypical nucleotide (i.e., a nucleotide other than deoxy-C, T, G or A in DNA, or other than C, U, G or A in RNA). For example, a nucleic acid molecule product of a transcription reaction can contain a combination of ribonucleotides and deoxyribonucleotides. In another example, a nucleic acid molecule can contain typically occurring nucleotides and mass modified nucleotides, or can contain typically occurring nucleotides and non-naturally occurring nucleotides.

ii. Conditioning

Prior to mass spectrometric analysis, nucleic acid molecules can be treated to improve resolution. Such processes are referred to as conditioning of the molecules. Molecules can be “conditioned,” for example to decrease the laser energy required for volatilization and/or to minimize fragmentation. A variety of methods for nucleic acid molecule conditioning are known in the art. An example of conditioning is modification of the phosphodiester backbone of the nucleic acid molecule (e.g., by cation exchange), which can be useful for eliminating peak broadening due to a heterogeneity in the cations bound per nucleotide unit. In another example, contacting a nucleic acid molecule with an alkylating agent such as alkyloidide, iodoacetamide, β-iodoethanol, or 2,3-epoxy-1-propanol, can transform a monothio phosphodiester bonds of a nucleic acid molecule into a phosphotriester bond. Likewise, phosphodiester bonds can be transformed to uncharged derivatives employing, for example, trialkylsilyl chlorides. Further conditioning can include incorporating nucleotides that reduce sensitivity for depurination (fragmentation during MS) e.g., a purine analog such as N7- or N9-deazapurine nucleotides, or RNA building blocks or using oligonucleotide triesters or incorporating phosphorothioate functions which are alkylated, or employing oligonucleotide mimetics such as PNA.

iii. Multiplexing

For some applications, simultaneous detection of more than one nucleic acid molecule fragment can be performed. In other applications, parallel processing can be performed using, for example, oligonucleotide or oligonucleotide mimetic arrays on various solid supports. “Multiplexing” can be achieved by several different methodologies. For example, fragments from several different nucleic acid molecules can be simultaneously subjected to mass measurement methods. Typically, in multiplexing mass measurements, the nucleic acid molecule fragments should be distinguishable enough so that simultaneous detection of the multiplexed nucleic acid molecule fragments is possible. Nucleic acid molecule fragments can be made distinguishable by ensuring that the masses of the fragments are distinguishable by the mass measurement method to be used. This can be achieved either by the sequence itself (composition or length) or by the introduction of mass-modifying functionalities into one or more nucleic acid molecules.

b. Other Measurement Methods

Additional mass measurement methods known in the art can be used in the methods of mass measurement, including electrophoretic methods such as gel electrophoresis and capillary electrophoresis, and chromatographic methods including size exclusion chromatography and reverse phase chromatography.

2. Mass Peak Characteristics

Using methods of mass analysis such as those described herein, information relating to mass of the target nucleic acid molecule fragments can be obtained. Additional information of a mass peak that can be obtained from mass measurements include signal to noise ratio of a peak, the peak area (represented, for example, by area under the peak or by peak width at half-height), peak height, peak width, peak area relative to one or more additional mass peaks, peak height relative to one or more additional mass peaks, and peak width relative to one or more additional mass peaks. Such mass peak characteristics can be used in the present sequence determination methods, for example, in a method of identifying the nucleotide sequence of a target nucleic acid molecule by comparing at least one mass peak characteristic of an amplification fragment with one or more mass peak characteristics of one or more reference nucleic acids.

3. Capture Oligonucleotide and Hybridization Conditions

In methods that include hybridization with capture oligonucleotides, typically the capture oligonucleotides have known nucleotide sequences. Further, the stringency of the hybridization conditions used when target nucleic acid fragments are contacted with capture oligonucleotides also are typically known. Knowledge of the sequence of the capture oligonucleotides and of the hybridization conditions can be used to provide information regarding the nucleotide sequence of the target nucleic acid fragment that hybridized to the capture oligonucleotide.

In methods for constructing the nucleotide sequence of a target nucleic acid molecule, the sequence of the capture oligonucleotide probe can be used to decrease the number of possible target nucleic acid sequences that are represented by a particular observed mass. When the sequence of the capture oligonucleotide is known, one skilled in the art can predict nucleotide sequence of target nucleic acid fragments that can hybridize to the capture oligonucleotide under particular hybridization conditions. In addition, one skilled in the art can predict nucleotide sequence of target nucleic acid fragments that likely do not hybridize to the capture oligonucleotide under particular hybridization conditions.

Possible presence of some nucleotide sequences and likely absence of other nucleotide sequences can assist in interpretation of mass observations. Observation of a particular mass can be used to determine the composition of a target nucleic acid fragment (e.g., the number of C's, G's, A's and T's in a DNA fragment) represented by that mass, but typically cannot, without more information, be used to determine the nucleotide sequence of the target nucleic acid fragment represented by that mass. Thus, typically, a particular mass observation can represent any of a variety of different target nucleic acid fragment nucleotide sequences. A mass observation can be supplemented with hybridization information (capture oligonucleotide and hybridization conditions), which can limit or reduce the number of likely nucleotide sequences represented by a particular mass observation. The limited or reduced number of likely nucleotide sequences can be used in methods of sequence construction or for comparison to a reference, as provided herein.

In an example, a four-nucleotide capture oligonucleotide can have the nucleotide sequence 5′ACTG 3′, and target nucleic acid fragments can be contacted with the capture oligonucleotide under high stringency conditions such that only target nucleic acid fragments that are completely complementary to the capture oligonucleotide hybridize to the capture oligonucleotide. Further to this example, masses of target nucleic acid fragments hybridized to this capture oligonucleotide are measured, and the compositions of the fragments are determined, where one mass is determined to have the composition A₃CTG. When mass (and thereby composition) and hybridization information are combined, the A₃CTG mass is predicted to contain one or more fragments having the nucleotide sequence AAACTG, AACTGA, or ACTGAA. Thus, the target nucleic acid molecule can contain one or more of the nucleotide sequences AAACTG, AACTGA, or ACTGAA.

In a similar example with the same capture oligonucleotide and hybridization conditions, no mass peak is observed that corresponds to the composition A₃CTG. This observation, when combined with hybridization information, can indicate that the target nucleic acid molecule is likely to not contain any of the nucleotide sequences AAACTG, AACTGA, or ACTGAA. In methods that include comparing observed and reference mass characteristics, the capture oligonucleotide sequence and hybridization conditions can be an additional source of information for matching a sample pattern and a reference pattern. For example, masses can be measured for a plurality of capture oligonucleotides in an array. A reference sequence can be observed or calculated to have a particular pattern of mass characteristics for each of the plurality of capture oligonucleotides, which can result in a two-dimensional pattern of mass vs. capture oligonucleotide. One or more reference patterns can be compared to the pattern of a sample to identify a target nucleic acid or to identify the nucleotide sequence, according to the methods provided herein.

4. Fragmentation Method

The method(s) used to fragment the target nucleic acid molecule can provide information that can be used in nucleotide sequence construction or other methods provided herein. In one example, fragmentation can be performed to yield target nucleic acid fragments having a known statistic size range. In another example, fragments can be “trimmed” after hybridization to the capture oligonucleotide to have either the same length as the capture oligonucleotide or a length that is typically only slightly larger than the capture oligonucleotide (e.g., when base-specific fragmentation trimming is preformed). Fragmentation methods also can limit the nucleotide sequence of one or more nucleotide loci in a fragment; typically this occurs when sequence specific cleavage (using, e.g., a base-specific RNase or a restriction endonuclease) is performed. Thus, fragmentation methods can be performed where the fragments produced have a known size (or size range), some known nucleotide sequence information, or both.

In addition to information about target nucleic acid fragments that can be known based on the fragmentation method(s) used, nucleotide sequence construction methods provided herein can take advantage of the information provided when overlapping fragments are produced by the fragmenatation method(s). The existence of overlapping fragments provides redundancy of information that can be used for constructing a nucleic acid sequence or for increasing the accuracy of the nucleic acid sequence construction. For example, a first and a second target nucleic acid fragment can arise from nucleotide portions that are adjacent to one another in a target nucleic acid; a third target nucleic acid fragment can contain a portion of the nucleotide sequence of the first target nucleic acid fragment and a portion of the nucleotide sequence of the second target nucleic acid fragment, and can be used to identify the first and second target nucleic acid fragments as adjacent nucleotide sequences and thereby serve to construct the nucleotide sequence of the target nucleic acid.

J. Nucleotide Sequence Construction

The information relating to target nucleic acid fragments, such as fragmentation method, mass measurement, mass peak characteristics, and the capture oligonucleotide (and hybridization conditions) to which the target nucleic acid fragment hybridized, can be used to construct the nucleotide sequence of the target nucleic acid molecule. For example, the methods of sequence construction can make use of the ability of mass spectrometry methods to separate and measure components of a sample according to the masses of the components. Also, the methods of sequence construction can make use of hybridization methods provided herein to reduce the complexity of nucleic acid fragments (e.g., the number and/or variability of nucleic acid fragments) in a sample while, optionally, still resulting in a sample with two or more nucleic acid fragments. Also, the methods of sequence construction can make use of the size and/or sequence of nucleic acid fragments formed by the fragmentation method(s), and can make use of the presence of overlapping nucleic acid fragments. By making use of these sources of information, a partial or entire nucleotide sequence of a nucleic acid molecule can be determined. The methods for nucleotide sequence construction can be used in methods of: long range de-novo sequencing, long range re-sequencing, long range SNP discovery, long range mutation discovery, bacteria typing using longer sequence regions (e.g., bacteria typing using full 16S rRNA gene based methods), multiplex sequencing (e.g., multiple shorter amplicons in one experiment), long range methylation analysis (using, e.g., specialized methylation chips with even less chip positions), human identification (using, e.g., one long region or multiple short regions), organism identification (using, e.g., one long region or multiple short regions), analysis of pathogen and non-pathogen mixtures, and quantitation of heterogenous nucleic acid mixtures.

1. Role of Information Relating to Target Nucleic Acid Fragments

The methods provided herein for constructing a nucleotide sequence can be based on the ability to predict or define limits for the nucleotide sequences of masses in a mass spectrum. For example, predicted sequences or sequence limitations to masses in a mass spectrum can be based on information such as: (1) the fragmentation method(s), (2) the capture oligonucleotide, and (3) mass measurement.

As provided herein, the fragmentation method(s) can be used to create any of a variety of nucleic acid fragments, for example, fragments having a nucleotide length within a particular range (e.g., ranging from 15-30 nucleotides in length), fragments cleaved at a particular base (e.g., base specific cleavage), fragments cleaved at one or more particular nucleotide sequences (e.g., fragments formed by digestion with sequence-specific endonuclease(s)), or fragments of the same length as the capture oligonucleotide (e.g., “trimmed” fragments). The resultant fragments have reduced complexity that are a function of the fragmentation method(s) used. For example, a pool of fragments with a particular range of nucleotide length (e.g., ranging 15-30 nucleotides in length) have reduced complexity relative to a pool of fragments without a particular range of nucleotide length (e.g., fragments of any length). The reduced complexity of the nucleotide fragments can be used to predict or define limits for the nucleotide sequences of fragments. For example, in base specific cleavage, all fragments have, at one end, a single particular nucleotide (the base-specifically cleaved nucleotide) and the remainder of the fragment have any of the remaining three nucleotides. The reduced complexity of the nucleotide fragments also can be used to limit the number of different nucleotide fragments that hybridize with a particular capture oligonucleotide and/or to limit the number of different nucleotide fragments measured by mass spectrometry. For example, if all fragments are the same length as the capture oligonucleotide, the number of fragments hybridized to the capture oligonucleotide and the number of fragments measured by mass spectrometry can be limited to only those complementary to the capture oligonucleotide.

As provided herein, the capture oligonucleotide can contain any of a variety of lengths of oligonucleotides, and can include universal bases and/or semi-universal bases. The number of different nucleotide fragments hybridized to each capture oligonucleotide can be controlled according to the length and composition of each capture oligonucleotide. For example, a longer capture oligonucleotide containing only typical nucleotides (e.g., A, C, G and T) can have fewer different nucleotide fragments hybridized thereto relative to a shorter capture oligonucleotide containing only typical nucleotides. In another example, a capture oligonucleotide containing only typical nucleotides can have fewer different nucleotide fragments hybridized thereto relative to a capture oligonucleotide of the same length containing one or more universal or semi-universal bases. The constraints on the number of different nucleotide fragments hybridized to a particular capture oligonucleotide can be used to predict or define limits for the nucleotide sequences of fragments. The constraints on the number of different nucleotide fragments hybridized to a particular capture oligonucleotide also can be used to limit the number of different nucleotide fragments measured by mass spectrometry.

Mass measurement can be used to determine the composition of one or more nucleotide fragments. For example, mass measurement can be used to determine the number of A's, T's, G's and C's present in a DNA fragment. The composition of a nucleotide fragment can be used to predict or define limits for the nucleotide sequences of fragments.

2. Methods for Sequence Construction

The information provided by, for example, fragmentation, capture oligonucleotide hybridization, and mass measurement, can be used in any of a variety of different methods provided herein to construct the nucleotide sequence of a target nucleic acid molecule. To construct the nucleotide sequence of the target nucleic acid molecule, the teachings provided herein can guide one skilled in the art to use known techniques for nucleotide sequence analysis by Sequencing By Hybridization along with known techniques for nucleotide sequence analysis by Mass Spectrometry. For example, the experimental data can be transformed into a subgraph of a de Bruijn graph by known methods; see, for example, Pevzner, J. Biomol. Struct. Dyn., 7:63-73 (1989). Eulerian paths in this graph can be searched for, where cycles and bulges have to be broken in advance, as is known in the art; see, for example, Pevzner et al., Proc. Natl. Acad. Sci. USA 98:9748-9753 (2001). Mass spectra can be used to uniquely identify the nucleotide composition of a nucleic acid fragment by methods known in the art; see, for example, Bocker, Lect. Notes Comp. Sci. 2812:476-487 (2003). Methods such as the branch-and-bound method for determining the nucleotide sequence from compomers can be used, as is known in the art, and exemplified in Bocker, Lect. Notes Comp. Sci. 2812:476-487 (2003). Complications to the branch-and-bound method caused by false negative peaks can be addressed by methods known in the art, as exemplified in S. Bocker, “Sequencing from compomers in the presence of false negative peaks” Technical Report 2003-07, Technische Fakultät der Universität Bielefeld, Abteilung Informationstechnik, 2003; also available at http://www.cebitec.uni-bielefeld.de/groups/ims/download/Preprint_{—}2003-07_WeightedSC_SBoecker.pdf.

In one exemplary method, a hypothetical nucleotide sequence of the target nucleic acid or a fragment thereof can be constructed, the fragmentation/hybridization/masses of the fragments can be predicted, and the predicted masses can be compared with observed masses to test whether the hypothetical nucleotide sequence may or may not be present. In another example, knowledge of the fragmentation/hybridization methods can be used to predict all possible masses that could be observed and to identify sequences that correspond to particular masses, this information can then be compared to observed masses to limit the number of different nucleotide sequences that can be present in the target nucleic acid molecule. Provided below are exemplary methods for using this information to construct a nucleotide sequence.

a. Hypothetical Sequence Testing

In one exemplary method for using fragmentation, hybridization and mass measurement information, a hypothetical nucleotide sequence of the target nucleic acid or a fragment thereof can be constructed, the fragmentation/hybridization/masses of the fragments can be predicted, and the predicted masses can be compared with observed masses to test whether the hypothetical nucleotide sequence may or may not be present. This method can be performed by constructing a hypothetical nucleotide sequence of a portion of the target nucleic acid molecule (e.g., one nucleotide fragment), and, upon determination of the nucleotide sequence of that portion, adding one or more additional hypothetical nucleotides to the portion, and testing whether the additional hypothetical nucleotides may or may not be present.

In one example, a target nucleic acid molecule can have a known nucleotide sequence at one or both ends (e.g., the 3′ end or the 5′ end, or both ends). This can be the case, for example, when the target nucleic acid molecule is amplified with a primer with a known nucleotide sequence. One or more hypothetical nucleotides can be added to the known sequence, and the presence of the hypothetical nucleotide(s) can be tested by reference to observed mass spectra. A mismatch between hypothetical and actual nucleotides result in the presence of hypothetical masses that are absent in the experimentally observed mass spectra, and/or the absence of hypothetical masses that are present in the experimentally observed mass spectra. Accordingly, the hypothetical nucleotide that yields predicted fragment masses that most closely match the experimentally observed masses can be identified as the nucleotide present at the corresponding position in the target nucleic acid molecule.

Presence or absence of numerous masses in each of a plurality of mass spectra can be used to determine which of the four nucleotides is present, and to provide redundancy of information, thereby increasing the probability of accurate sequence determination. For example, the identity of a nucleotide at a particular nucleotide position can be determined by comparison of predicted masses and observed masses for a single mass spectrum; in addition to such a determination, further information confirming or refuting the determination can be obtained by reference to one or more additional mass spectra. By referring to multiple mass spectra, the number of observations used to identify a particular nucleotide can be increased, and, therefore, the probability of accurate nucleotide identification can be increased.

One exemplary method for sequence construction based on nucleotide hypothesis testing is as follows:

(1) Assign a hypothetical nucleotide at one or more particular positions;

(2) Predict fragments containing that nucleotide(s) according to the fragmentation method(s);

(3) For each capture oligonucleotide, predict whether or not there is hybridization of the predicted fragments to the capture oligonucleotide;

(4) Calculate masses/composition of the hybridized fragments for each capture oligonucleotide; and

(5) Compare predicted masses to observed masses;

a match between predicted and observed masses can identify the hypothetical nucleotide(s) as the actual nucleotide(s) in the target nucleic acid molecule nucleotide sequence.

This method can, if desired, be repeated for all four typically occurring nucleotides (e.g., A, G, C and T for DNA) at each nucleotide position, and the nucleotide for which the predicted masses most closely match the observed masses can be selected as the nucleotide present at that position in the target nucleic acid molecule. A single or multiple nucleotide positions can be simultaneously tested by this method, and the number of nucleotide positions to be simultaneously tested can be determined according to the number of observations (e.g., the number of masses present and the number of masses absent), the mass spectra (e.g., the number of different sequences that can be present in a mass spectrum), and the length of the target nucleic acid molecule, according to the guidelines provided herein and methods known in the art.

In a specific illustrative example of sequence construction based on nucleotide hypothesis testing, a target oligonucleotide with the (unknown) nucleotide sequence ACATGAGCTTACAAC (SEQ ID NO: 1) can be fragmented to yield fragments 5-7 nucleotides in length. Next, the nucleic acid fragments can be hybridized by capture oligonucleotides having a hybridization region of four semi-universal bases (e.g., bases that bind only pyrimidines (Y) or only purines (R)). Next, the hybridized fragments can be detected by mass spectrometry. For purposes of this example, the sequence of the first seven nucleotides of the target oligonucleotide is known to be ACATGAG. The eighth nucleotide can be tentatively assigned to be any of the four possible typically occurring nucleotides, for example, a “T.” Masses can be predicted for each mass spectrum measured for each different capture oligonucleotide sequence, based on an oligonucleotide containing the sequence ACATGAGT. For example, when “T” is tentatively assigned at that nucleotide position, the mass spectrum for a capture oligonucleotide probe with the sequence RYYY are predicted to contain a mass corresponding to the composition T₂G₂A, T₂G₂A₂, and T₂G₂A₂C. For the nucleotide sequence ACATGAGCTTACAAC (SEQ ID NO: 1), only T₂G₂A₂C are experimentally observed for this capture oligonucleotide. Similarly, the presence of a “G” would yield three predicted masses, none of which are present experimentally for this capture oligonucleotide. When the eight position is predicted to be “A,” two of three predicted mass are present experimentally, and when the eighth position is predicted to be “C” all corresponding experimental masses are observed. Thus, “C” provides the closest match. To further confirm the presence of “C” at this position, masses from spectra of one or more other capture oligonucleotides can be compared. For example, if an “A” is present, the mass spectrum from a capture oligonucleotide with the sequence YYYY includes a mass corresponding to TG₂A₂. No such mass is experimentally observed; but the mass spectrum for the capture oligonucleotide YYYR has a mass corresponding to the composition TG₂AC, indicating that “C” may be/is present at that position.

In this example, 16 different capture oligonucleotides can be used, and each capture oligonucleotide can hybridize to several nucleic acid fragments containing overlapping sequences (e.g., when fragments are 5-7 nucleotides in length, 9 different fragments with overlapping sequences can hybridize to the same 4 nucleotide long capture oligonucleotide). Thus, in this example, up to 9 different masses of a single mass spectrum can provide information on the identity of a nucleotide at a particular nucleotide position, and sixteen different mass spectra can be collected. Accordingly, a large amount of information can be used to identify the nucleotide at each nucleotide position of this target oligonucleotide.

b. Limiting Possible Sequences

In one example, the fragmentation method(s) and composition of the capture oligonucleotide can be used to define or limit the number of possible nucleotide sequences that can be represented in a particular mass of a mass spectrum of nucleotide fragments hybridized to the capture oligonucleotide, and also can be used to define or limit the number of possible masses that can be present in a mass spectrum of nucleotide fragments hybridized to the capture oligonucleotide. For example, a fragmentation method that cleaves all fragments to a length of 8 nucleotides limits the number of different nucleotide sequences that can be present to 48, and the number of different masses possible in a mass spectrum is even further limited. A capture oligonucleotide that hybridizes to a specific 4-nucleotide sequence at the 3′ end of the nucleotide fragment, further limits the number of possible nucleotide sequences that can be present (at a particular capture oligonucleotide position) to 44, and the number of different masses possible in a mass spectrum is even further limited.

These limits can be applied to an experimentally measured mass spectrum to yield limits to the possible nucleotide sequence of the target nucleic acid molecule. The limits can be either positive (e.g., a particular nucleotide sequence is or may be present in the target nucleic acid molecule) or negative (e.g., a particular nucleotide sequence is not present in the target nucleic acid molecule). For example, a mass of a fragment resultant from the above exemplary fragmentation and capture oligonucleotide conditions can be limited to correspond to 24 or fewer possible nucleotide sequences, resulting in limiting an 8-nucleotide segment of the target nucleic acid molecule to one of 24 or fewer nucleotide sequences. Also, the absence of any fragments having a particular mass can indicate that no nucleotide sequence that would yield such a mass is present in the target nucleic acid molecule. In further refinements, mass spectra from numerous different capture oligonucleotides can be compared, and negative and positive limits from multiple mass spectra can reduce the number of possible sequences that can be present at particular observed masses.

When the number of observations (an observation including presence of a particular mass or absence of a particular mass) is sufficiently large and the mass spectra (e.g., the number of different sequences that can be present in each mass spectrum) sufficiently simplified relative to the nucleotide sequence to be constructed (as can be determined by known methods according to the teachings provided herein), the nucleotide sequence of the target nucleic acid molecule can be constructed in part or in whole. For example, in some cases, observed nucleotide fragment compositions (which can be determined, for example, from observed masses) can have nucleotide sequences assigned thereto; and when a sufficient number of nucleotide fragments, particularly overlapping fragments, have nucleotide sequences assigned, the entire nucleotide sequence of the target nucleic acid molecule can thereby be constructed. In another example, no observed nucleotide fragment composition can have a nucleotide sequence assigned thereto; nevertheless, limits to possible nucleotide sequences of the fragments can be used to determine the sequence of the target nucleic acid molecule, by, for example, providing sufficient limits to determine overlap between fragments and providing sufficient limits to determine the sequences of the fragments based on the overlap between fragments. In yet another example, fragments having assigned nucleotide sequences can be used in conjunction with fragments with unassigned nucleotide sequences but having limits to their nucleotide sequences.

One exemplary method for sequence construction based on limiting possible sequences of nucleotide fragments and/or the target nucleic acid molecule can be performed according to the following steps:

(1) Define or establish limits for fragment products of nucleic acid fragmentation;

(2) Define or establish limits for nucleic acid fragments that can hybridize to each particular capture oligonucleotide;

(3) Predict possible masses that can be observed in a mass spectrum of nucleotide fragments hybridized to a capture oligonucleotide;

(4) Create limiting rule set for possible nucleotide sequences that could be present in a particular observed mass; and

(5) Compare observed masses to the rule set to identify possible sequences that could be present and/or to identify sequences that are not present.

3. Guidelines for Determining Robustness of Method

One skilled in the art can determine the length of the target nucleic acid molecule whose sequence can be constructed and/or the degree of probability that a sequence determination is correct, according to factors that are a function of the methods provided herein. Additionally, one skilled in the art can design the methods provided herein according to the length of the target nucleic acid molecule whose sequence is to be constructed and/or the desired degree of probability that a sequence determination is correct. For example, the methods provided herein can govern the amount of experimental information available for sequence construction and the degree to which the experimental information represents unique nucleotide sequences present or absent in the target nucleic acid molecule.

For example, the methods provided herein can govern the number of different mass observations that can be used in nucleotide sequence construction. A mass observation can be, for example, a mass present in a mass spectrum, or a mass absent from a mass spectrum (e.g., absence of a peak at a mass of a possible nucleotide fragment). The number of mass observations for a mass spectrum can be influenced by the fragmentation method(s) used, and the hybridization method used (e.g., hybridization conditions and the sequence of the capture oligonucleotide). For example, fragmentation of a target nucleic acid molecule that yields only fragments that are 10 nucleotides in length can decrease the number of mass observations relative to fragmentation of a target nucleic acid molecule that yields fragments that are 5-15 nucleotides in length. The number of mass observations also can be influenced by the number of mass spectra collected for different hybridization reactions (e.g., different hybridization conditions and/or different capture oligonucleotide sequences).

The methods provided herein also can govern the number and/or variability of nucleotide sequences with the same mass that can be represented in the same mass spectrum. For example, the fragmentation and hybridization methods provided herein can influence the number of different nucleotide sequences that have the same nucleotide composition and can be present in the same mass spectrum, and thereby are represented in the same mass peak of a mass spectrum.

Methods are known to those skilled in the art for determining the experimental information that can be obtained, for example, the number of observations and the number of different nucleotide sequences that can be represented in the same observation. Upon determining the experimental information that can be obtained, one skilled in the art can estimate the nucleic acid molecule length and/or degree of probability of nucleotide sequence determination. Alternatively, based on the desired target nucleic acid molecule length and/or desired degree of probability of nucleotide sequence determination, one skilled in the art can design the number and type of fragmentation method(s) and/or hybridization reactions for accomplishing the desired result.

K. Identifying a Nucleotide Sequence by Mass Pattern

In another embodiment, a method is provided herein for identifying a nucleotide sequence of a target nucleic acid molecule, comprising:

(a) hybridizing fragments of a target nucleic acid molecule to a capture oligonucleotide probe, wherein two or more different target nucleic acid fragments hybridize to the capture oligonucleotide probe;

(b) measuring the mass of the target nucleic acid fragments hybridized to the capture nucleic acid probe;

(c) comparing the sample masses with one or more reference mass patterns;

(d) identifying a reference mass pattern that matches the sample masses;

whereby a match between the sample masses and a reference mass pattern identifies a nucleotide sequence in the target nucleic acid molecule as corresponding to the reference nucleotide sequence. In such methods, two or more characteristics of mass peaks can be used to identify the sequence in the target nucleic acid. In such a method of identification, the collection of two or more characteristics of mass peaks is referred to as a “pattern”.

In the methods provided herein, a particular nucleotide sequence can give rise to a pattern of masses that serves as a unique signature of that nucleotide sequence. For example, a particular nucleotide sequence can give rise to a pattern of masses that is formed only when the target nucleic acid contains that nucleotide sequence. In such situations, nucleotide sequence constructions are not needed to identify the nucleotide sequence—the nucleotide sequence can be identified simply by matching the observed pattern with a reference pattern where the reference pattern corresponds to a specific nucleotide sequence.

The pattern of masses can be present in a single mass spectrum, or can be present in the mass spectrum of two or more different hybridization reactions. The reference pattern can be a calculated pattern or an experimentally observed pattern. In instances where the reference pattern is experimentally observed, nucleotide sequence identification is not influenced by the presence of reproducible error (e.g., an error in a mass spectrum in which a peak that is calculated to be present or absent is reproducibly absent or present, respectively).

In some embodiments, sequence identification by pattern matching can be combined with the nucleotide sequence construction methods provided herein. For example, the nucleotide sequence of a section of a target nucleic acid molecule can be determined by pattern matching, and the location of that section in the target nucleic acid and/or the nucleotide sequence of the remainder of the target nucleic acid molecule can be determined by nucleotide sequence construction methods. In other embodiments, sequence identification by pattern matching can be used to identify the entire nucleotide sequence of the target nucleic acid molecule.

In some instances, such as re-sequencing and SNP analysis, it can be possible that a previously known sequence (e.g., public database sequence) exists for the target nucleic acid molecule, however, the sequence of the particular target nucleic acid of interest is not known. In other cases, target nucleic acid fragment mass patterns can be known for a particular nucleotide sequence. In either case, it is possible to identify a nucleotide sequence in a target nucleic acid by measuring the pattern of masses of the target nucleic acid fragments that hybridize to one or more capture oligonucleotides, and comparing the pattern to either calculated or experimentally determined mass patterns.

The mass peaks to be identified can have three or more identifying characteristics, including position on the capture oligonucleotide array (i.e., the particular capture oligonucleotide with which the target fragment hybridizes and when the sequence of the capture oligonucleotide is known, the sequence to which the target nucleic acid fragment hybridizes), measured mass, and signal to noise ratio of the mass measurement. It is contemplated herein that as few as 1 or as few as 2 identifying characteristics of a mass peak can be used in methods of nucleotide sequence determination by mass pattern matching.

In analysis of a known sequence (e.g., in resequencing or genotyping methods), calculated mass patterns or experimentally determined mass patterns can be used to identify one or more mass peak characteristics that can identify a nucleotide sequence in a target nucleic acid. For example, SNP analysis can be carried out by determining one or more peaks that indicate the presence or absence of a particular nucleotide at the SNP position in question. Thus, identifying the presence or absence of one or more indicative mass peaks can serve to identify the nucleotide at the SNP position in question, without requiring nucleotide sequence construction methods to determine all or any of the nucleotide sequence of the target nucleic acid molecule.

Calculations of fragmentation and hybridization patterns can identify mass peaks which can be used to predict a mass pattern or a mass peak characteristics pattern. Such a method can generate any or all of the characteristics of mass peaks, including presence or absence of a fragment at a particular site on the capture oligonucleotide array, mass of a fragment, and signal to noise ratio of a mass peak. In some instances, by repeating these calculations for different nucleotide sequences of the same positions in question, it is possible to generate several differing (and mutually exclusive) collections of one or more mass peaks indicative of different nucleotide sequences at the one or more nucleotide portions on the target nucleic acid.

Experimental analysis of sample target nucleic acid fragments can generate mass peaks which can be compared to one or more collections of the calculated sequence-indicative mass peaks, and the one or more collections of theoretically calculated sequence-indicative mass peaks can be correlated to the experimental mass peaks. The entire sequence or part of the sequence of the sample target nucleic acid can then be identified as the reference sequence corresponding to the collection of calculated sequence-indicative mass peaks that most closely correlates to experimental mass peaks, provided, optionally, that the correlation is above a user-defined threshold amount. A similar correlation can be made between experimentally derived reference mass patterns and mass patterns of the sample target nucleic acid molecule.

Correlation of sample peaks and reference peaks can be carried out in any of a variety of ways known to those of skill in the art. In a simple example, one reference mass present for a particular capture oligonucleotide may be present in only one of a variety of reference mass peak patterns. If that same mass is detected for a sample target nucleic acid molecule, at least part of the nucleotide sequence for the target nucleic acid molecule can be identified as the nucleotide sequence corresponding to the reference mass peak. Correlations between sample peaks and reference peaks also can be carried out using statistical methods that consider a plurality of peaks, including regression methods such as linear or non-linear regression, and using other methods known for data correlation.

In one embodiment, a user can define a threshold which sets a minimum correlation required for the reference nucleic acid to, with sufficient likelihood, identify a nucleotide sequence in a target nucleic acid. When no correlation occurs that is above the threshold value, none of the reference nucleic acids can, with sufficient likelihood, identify a nucleotide sequence in a target nucleic acid.

In one embodiment, the mass pattern of target nucleic acid fragments hybridized to a capture probe in a single position in the array can serve to identify one or more sequences or portions of a target nucleic acid. For example, when the sample target nucleic acid is a chromosome from an organism, and the target nucleic acid is being tested for a particular gene or sequence for determination of, for example, gene expression, genotype, species and variety the mass pattern of target nucleic acid fragments hybridized to a capture probe in a single position in the array (e.g., all target nucleic acid fragments are hybridized to capture oligonucleotide probes which all have the same nucleotide sequence) can indicate the particular gene expressed, genotype, species, or variety, or can indicate that the target nucleic acid does not correspond to a particular gene expressed, genotype, species, or variety.

In other embodiments, the mass pattern of target nucleic acid fragments hybridized to a plurality of capture probe array positions can serve to identify a nucleotide sequence in a target nucleic acid, where the target nucleic acid fragments are hybridized to capture probes located in 500 or fewer positions in the array, 250 or fewer positions in the array, 100 or fewer positions in the array, 75 or fewer positions in the array, 50 or fewer positions in the array, 25 or fewer positions in the array, 20 or fewer positions in the array, 15 or fewer positions in the array, 10 or fewer positions in the array, 8 or fewer positions in the array, 6 or fewer positions in the array, 5 or fewer positions in the array, 4 or fewer positions in the array, 3 or fewer positions in the array, or 2 or fewer positions in the array.

In methods that do not require nucleotide sequence construction, generating overlapping target nucleic acid fragments can be used, but is not required. For example, in resequencing methods or methods for identifying the sequence of an SNP, non-overlapping target nucleic acid fragments can be generated, and all or part of the nucleotide sequence can be determined. In applications such as SNP identification, as few as a single target nucleic acid fragment can be used to indicate the nucleotide sequence of the target nucleic acid that the SNP position.

L. Identifying a Portion of a Target Nucleic Acid

In another embodiment, a method is provided herein for identifying a portion of a target nucleic acid, comprising:

(a) hybridizing fragments of the target nucleic acid to a capture oligonucleotide probe, wherein two or more different target nucleic acid fragments hybridize to the capture oligonucleotide probe;

(b) measuring the mass of the target nucleic acid fragments hybridized to the capture nucleic acid probe; and

(c) comparing the masses with the mass of fragments of a reference nucleic acid molecule;

whereby a correlation between one or more sample masses and one or more reference masses identifies the portion of a target nucleic acid as corresponding to the reference nucleic acid molecule. In such a method of identification, the collection of two or more characteristics of mass peaks is referred to as a “pattern”.

In one embodiment, it is possible to identify one or more portions of a target nucleic acid using a pattern of the masses of target nucleic acid fragments that hybridize to one or more capture oligonucleotides, without the need to determine the entire nucleotide sequence of the target nucleic acid. In another embodiment, one or more portions of a target nucleic acid are identified without determining any of the nucleotide sequence of the target nucleic acid.

In some cases, reference nucleic acid mass patterns can be known for demonstrating where a target nucleic acid molecule or fragment thereof is located, even if the sequence of the target nucleic acid is not known. For example, a chromosome can have a target nucleic acid fragment map, analogous to an RFLP or AFLP map, but all or only a subset of the chromosome may a have known nucleotide sequence. Whether the nucleotide sequence is known or not, it is possible to identify a portion of a target nucleic acid molecule by measuring the pattern of masses of the target nucleic acid fragments that hybridize to one or more capture oligonucleotides, and comparing the pattern to either calculated (in the case of known sequences) or experimentally measured mass patterns.

When the sequence of the region in question is unknown, identification of one or more portions of a target nucleic acid can nevertheless be accomplished by comparing one or more mass peaks of target nucleic acid fragments with one or more mass peaks from one or more reference nucleic acids. This method can be similar to traditional DNA fingerprinting methods in which one or more gel electrophoresis bands for an unknown sample is compared to one or more gel electrophoresis bands of one or more known or reference samples. In the present methods, for example, one or more of the three characteristics of mass peaks measured from a sample target nucleic acid (i.e., position on array, mass, and signal to noise) can be compared to one or more characteristics of mass peaks measured from one or more reference nucleic acids, and the mass peaks of the one or more references can be correlated to the sample target nucleic acid mass peaks. The portion of the sample target nucleic acid is then identified as corresponding to a portion of the reference nucleic acid having one or more mass peaks that most closely correlate to the sample target nucleic acid mass peaks, and optionally, provided that the correlation is above a user-defined threshold amount. Thus, identification of one or more portions of a target nucleic acid can be accomplished by identifying a particular reference nucleic acid as having the same mass pattern, even if neither the sequence nor location of the portions in question is known.

In one embodiment, the mass pattern of target nucleic acid fragments hybridized to a capture probe in a single position in the array can serve to identify a portion of a target nucleic acid. For example, when the sample target nucleic acid is a chromosome from an organism, and the target nucleic acid is being tested, for example, for gene expression, genotype, species and variety, the mass pattern of target nucleic acid fragments hybridized to a capture probe in a single position in the array, can indicate the particular gene expressed, genotype, species, or variety, or can indicate that the target nucleic acid does not correspond to a particular gene expressed, genotype, species, or variety.

In other embodiments, the mass pattern of target nucleic acid fragments hybridized to a plurality of capture probes can serve to identify a portion of a target nucleic acid, where the target nucleic acid fragments are hybridized to capture probes located in 500 or fewer positions in the array, 250 or fewer positions in the array, 100 or fewer positions in the array, 75 or fewer positions in the array, 50 or fewer positions in the array, 25 or fewer positions in the array, 20 or fewer positions in the array, 15 or fewer positions in the array, 10 or fewer positions in the array, 8 or fewer positions in the array, 6 or fewer positions in the array, 5 or fewer positions in the array, 4 or fewer positions in the array, 3 or fewer positions in the array, or 2 or fewer positions in the array.

In methods that do not require nucleotide sequence construction, generating overlapping target nucleic acid fragments can be used, but is not required. For example, an organism, strain or species can be identified using a pattern of target nucleic acid fragments where the each of the two or more mass peak characteristics used in the pattern arise from target nucleic acid fragments that represent non-adjacent sequences in the target nucleic acid; this pattern can be compared to one or more reference nucleic acid patterns and the organism, strain or species identified by correlating the sample pattern with the one or more reference patterns.

M. Applications:

The methods disclosed herein can be used to yield information about a target nucleic acid for a variety of purposes. The applications disclosed below provide exemplary use of the herein-disclosed methods. One skilled in the art understands that the applications described below can be performed using methods of constructing the nucleotide sequence of a target nucleic acid, and also can be carried out using methods for identifying a portion of a target nucleic acid, such as methods that entail analysis of target nucleic acid mass peak patterns.

1. Long Range Resequencing

In addition to the long range de-novo sequencing methods described above, the sequencing methods provided herein also can be used for long range re-sequencing. The dramatically growing amount of available genomic sequence information from various organisms increases the need for technologies allowing large-scale comparative sequence analysis to correlate sequence information to function, phenotype, or identity. The application of such technologies for comparative sequence analysis can be widespread, including, for example, SNP discovery and sequence-specific identification of pathogens. Therefore, resequencing and high-throughput mutation screening technologies are critical to the identification of mutations underlying disease, as well as the genetic variability underlying differential drug response, and differential response to treatment regimens.

Several approaches have been developed in order to satisfy these needs. Technology for high-throughput DNA sequencing includes DNA sequencers using electrophoresis and laser-induced fluorescence detection. Electrophoresis-based sequencing methods have inherent limitations for detecting heterozygotes and are compromised by GC compressions. Thus a DNA sequencing platform that produces digital data without using electrophoresis overcomes these problems. Matrix-assisted laser desorption/ionization time-of-flight mass spectrometry (MALDI-TOF MS) measures DNA fragments with digital data output. The methods of specific cleavage fragmentation analysis provided herein allow for high-throughput, high speed and high accuracy in the elucidation of nucleic acid sequence relative to a reference sequence. This approach makes it possible to routinely use MALDI-TOF MS sequencing for accurate sequence corrections as well as mutation detection, such as screening for founder mutations in BRCA1 and BRCA2, which are linked to the development of breast cancer.

Resequencing methods can be carried out using a variety of methods disclosed herein for target nucleic acid analysis. For example, resequencing can be carried out using sequence construction methods which can be used to determine the nucleotide sequence of large segments of a nucleic acid. In another example, methods of identifying a portion of a target nucleic acid can be used; for example, where the target nucleic acid can vary from a known or reference nucleic acid by only a small percentage (e.g., 5% or less), methods such as mass peak pattern analysis can be used to identify the nucleotide positions that vary and the identity of the nucleotides at the variant nucleotide positions. Thus, for example, when public database nucleotide sequences contain errors, a variety of the methods disclosed herein can be used to correct one or more of the errors.

2. Long Range Detection of Mutations/Sequence Variations

An object herein is to provide improved comparative nucleic acid sequencing methods useful for identifying the genomic basis of disease and markers thereof. The sequence variation candidates identified by the methods provided herein include sequences containing sequence variations that are polymorphisms. Polymorphisms include both naturally occurring, somatic sequence variations and those arising from mutation. Polymorphisms include but are not limited to: sequence microvariants, including SNPs, where one or more nucleotides in a localized region vary from individual to individual, insertions and deletions which can vary in size from one nucleotide to millions of bases, and microsatellites or nucleotide repeats which vary by numbers of repeats. Nucleotide repeats include homogeneous repeats such as dinucleotide, trinucleotide, tetranucleotide or larger repeats, where the same sequence is repeated multiple times, and also heteronucleotide repeats where sequence motifs are found to repeat. For a given locus the number of nucleotide repeats can vary depending on the individual.

A polymorphic marker or site is the locus at which divergence occurs. Such site can be as small as one base pair (e.g., a SNP). Polymorphic markers include, but are not limited to, restriction fragment length polymorphisms (RFLPs), variable number of tandem repeats (VNTR's), hypervariable regions, microsatellites, dinucleotide repeats, trinucleotide repeats, tetranucleotide repeats and other repeating patterns such as satellites, and minisatellites, simple sequence repeats and insertional elements, such as Alu. Polymorphic forms also are manifested as different mendelian alleles for a gene. Polymorphisms can be observed by differences in proteins, protein modifications, RNA expression modification, epigenomic differences, DNA and RNA methylation, regulatory factors that alter gene expression and DNA replication, and any other manifestation of alterations in genomic nucleic acid or organelle nucleic acids.

Furthermore, numerous genes have polymorphic regions. Since individuals have any one of several allelic variants of a polymorphic region, individuals can be identified based on the type of allelic variants of polymorphic regions of genes. This can be used, for example, for forensic purposes. In other situations, it is crucial to know the identity of allelic variants that an individual has. For example, allelic differences in certain genes, for example, major histocompatibility complex (MHC) genes, are involved in graft rejection or graft versus host disease such as in bone marrow transplant. Accordingly, it highly desirable to develop rapid, sensitive, and accurate methods for determining the identity of allelic variants of polymorphic regions of genes or genetic lesions. A method or a kit as provided herein can be used to genotype a subject by determining the identity of one or more allelic variants of one or more polymorphic regions in one or more genes or chromosomes of the subject. Genotyping a subject using one or more of the methods provided herein can be used for forensic or identity testing purposes and the polymorphic regions can be present in, for example, mitochondrial genes or can be short tandem repeats.

Single nucleotide polymorphisms (SNPs) are generally biallelic systems, that is, there are two alleles that an individual can have for any particular marker. This means that the information content per SNP marker is relatively low when compared to microsatellite markers, which can have upwards of 10 alleles. SNPs also tend to be very population-specific; a marker that is polymorphic in one population may not be very polymorphic in another. SNPs, found approximately every kilobase (see Wang et al. Science 280:1077-1082 (1998)), offer the potential for generating very high density genetic maps, which is useful for developing haplotyping systems for genes or regions of interest, and because of the nature of SNPs, they can in fact be the polymorphisms associated with the disease phenotypes under study. The low mutation rate of SNPs also makes them excellent markers for studying complex genetic traits.

Much of the focus of genomics has been on the identification of SNPs, which are important for a variety of reasons. They allow indirect testing (association of haplotypes) and direct testing (functional variants). They are the most abundant and stable genetic markers. Common diseases are best explained by common genetic alterations, and the natural variation in the human population aids in understanding disease, therapy and environmental interactions.

3. Multiplex Sequencing

Also contemplated herein, are methods for the high-throughput elucidation of nucleic acid sequences from a plurality of target nucleic acid sequences. Multiplexing refers to the simultaneous elucidation of more than one target nucleic acid sequence. Methods for performing multiplexed reactions, particularly in conjunction with mass spectrometry, are known (see, e.g., U.S. Pat. Nos. 6,043,031, 5,547,835 and International PCT application No. WO 97/37041).

Multiplexing can be performed, for example, for multiple shorter regions of the same target nucleic acid sequence using multiple shorter amplicons of the target nucleic acid in one experiment. Multiplexing provides the advantage that a plurality of target-nucleic acids can be sequenced in as few as a single mass spectrum, as compared to having to perform a separate mass spectrometry analysis for each individual target nucleic acid sequence. The methods provided herein lend themselves to high-throughput, highly-automated processes for elucidating nucleic acid sequences with high speed and accuracy.

Multiplexing can be used to determine the entire sequence of a target nucleic acid, to determine the sequence of at least one nucleotide, but not all nucleotides of a target nucleic acid, to identify one or more portions of a target nucleic acid, or to identify presence, or presence and relative concentration of one or more particular target nucleic acids in a sample containing plurality of different target nucleic acids. In one embodiment, the target nucleic acids are two or more mRNA nucleic acids or amplified nucleic acids formed using templates of two or more mRNA nucleic acids. In such a method, the gene expression profile of one or more cells, including a tissue sample or a blood or bone marrow sample, can be examined. For example, two or more mass peaks can be indicative of expression of two or more mRNAs, and measurement of the two or more mass peaks can reveal whether or not each of the mRNAs are present in the target nucleic acid sample, and the level at which the mRNAs are present in the target nucleic acid sample. Such methods can be used to examine the expression levels of any of a variety of mRNAs, including, for example, oncogenes and other genes indicative of the neoplastic or metastatic state of a cell, genes encoding cell-surface proteins, genes associated with a genetic disorder, mRNAs indicative of infection by a pathogen or other disease state of a cell and genes associated with activated cytotoxic cells. Such methods also can be used to determine the expression levels of one or more genes in a variety of different samples including, for example, different cell types, different tissue types, different organisms, different strains, different species, or new cell types, new tissue types, new organisms, new strains and new species. Determination of expression levels in different samples can be used, for example, to determine the metastatic state of cells, to diagnose a subject, including a patient with a genetic, infectious, autoimmune or neoplastic disease; to distinguish between cell types, tissue types, strain types or organism types; to determine linkage in expression between two or more genes; or to determine a correlation between gene expression and cell morphology such as mitotic or meiotic state of a cell.

A mixture of biological samples from any two or more biomolecular sources can be pooled into a single mixture for analysis herein. For example, the methods provided herein can be used for sequencing multiple copies of a target nucleic or amino acids from different sources, and therefore detect sequence variations in a target nucleic or amino acid in a mixture of nucleic acids in a biological sample. A mixture of biological samples also can include but is not limited to nucleic acid from a pool of individuals, or different regions of nucleic acid from one or more individuals, or a homogeneous tumor sample derived from a single tissue or cell type, or a heterogeneous tumor sample containing more than one tissue type or cell type, or a cell line derived from a primary tumor. Also contemplated are methods, such as haplotyping methods, in which two mutations in the same gene are detected.

4. Long Range Methylation Pattern Analysis

The methods provided herein can be used to elucidate nucleic acid sequence variations that are epigenetic changes in the target sequence, such as a change in methylation patterns in the target sequence. Analysis of cellular methylation is an emerging research discipline. The covalent addition of methyl groups to cytosine is primarily present at CpG dinucleotides (microsatellites). Although the function of CpG islands not located in promoter regions remains to be explored, CpG islands in promoter regions are of special interest because their methylation status regulates the transcription and expression of the associated gene. Methylation of promotor regions leads to silencing of gene expression. This silencing is permanent and continues through the process of mitosis and meiosis. Due to its significant role in gene expression, DNA methylation has an impact on developmental processes, imprinting and X-chromosome inactivation, as well as tumor genesis, aging, and also suppression of parasitic DNA. Methylation is thought to be involved in the oncogenesis of many widespread tumors, such as lung, breast, and colon cancer, and in leukemia. There also is a relation between methylation and protein dysfunctions (long Q-T syndrome) or metabolic diseases (transient neonatal diabetes, type 2 diabetes).

Bisulfite treatment of genomic DNA can be utilized to analyze positions of methylated cytosine residues within the DNA. Treating nucleic acids with bisulfite deaminates cytosine residues to uracil residues, while methylated cytosine remains unchanged. Thus, for example, by comparing the sequence of a target nucleic acid that is not treated with bisulfite to the sequence of the nucleic acid that is treated with bisulfite in the methods provided herein, the degree of methylation in a nucleic acid as well as the positions where cytosine is methylated can be deduced. Such comparisons between treated and untreated target nucleic acids can be accomplished by any of a variety of methods. For example, the untreated target nucleic acid could be a previously known sequence where the mass peaks generated from the untreated target nucleic acid are calculated and are not determined experimentally. In addition, the untreated target nucleic acid sequence mass peaks can be determined experimentally by carrying out fragmentation and mass peak analysis without bisulfite treatment. In another method, the complementary strands of the same treated target nucleic acid can serve to identify methylated cytosines. This method is based on the base pair mismatches that arise when bisulfite is used to convert cytosine to uracil. After treatment with bisulfite, the methylated double stranded target nucleic acid contains one or more G-U mismatches. By determining the sequence of both complementary strands, the presence of G-U mismatches can be used to indicate presence of an unmethylated cytosine at the uracil position, and the presence of G-C matched base pairs can be used to indicate the presence of a methylated cytosine.

Methylation analysis via restriction endonuclease reaction is made possible by using restriction enzymes which have methylation-specific recognition sites, such as Hpa II and MSP I. The basic principle is that certain enzymes are blocked by methylated cytosine in the recognition sequence. Once this differentiation is accomplished, subsequent analysis of the resulting fragments can be performed using the methods as provided herein.

These methods can be used together in combined bisulfite restriction analysis (COBRA). Treatment with bisulfite causes a loss in BstU I recognition site in amplified PCR product, which causes a new detectable fragment to appear on analysis compared to untreated sample. The fragmentation-based sequencing methods provided herein can be used in conjunction with specific cleavage of methylation sites to provide rapid, reliable information on the methylation patterns in a target nucleic acid sequence.

5. Organism Identification

Methods provided herein can be used to identify an organism or to distinguish an organism as different from other organisms. In one embodiment, the identification of a human sample can be performed (e.g., one long region or multiple short regions). Polymorphic STR loci and other polymorphic regions of genes are sequence variations that are extremely useful markers for human identification, paternity and maternity testing, genetic mapping, immigration and inheritance disputes, zygosity testing in twins, tests for inbreeding in humans, quality control of human cultured cells, identification of human remains, and testing of semen samples, blood stains and other material in forensic medicine. Such loci also are useful markers in commercial animal breeding and pedigree analysis and in commercial plant breeding. Traits of economic importance in plant crops and animals can be identified through linkage analysis using polymorphic DNA markers. Efficient and accurate fragmentation-based nucleic acid sequencing methods, and the methods provided herein for identifying a portion of a target nucleic acid can be used for determining the identity of such loci. The target-nucleic acid (e.g., genomic DNA) can be obtained from one long target nucleic acid region and/or multiple short target nucleic acid regions.

In other embodiments, methods can be used for identifying non-human organisms such as non-human mammals, birds, plants, fungi and bacteria.

6. Pathogen Identification and Typing

Also contemplated herein is a process or method for identifying strains of microorganisms using the fragmentation and hybridization-based methods provided herein. The microorganism(s) are selected from a variety of organisms including, but not limited to, bacteria, fungi, protozoa, ciliates, and viruses. The microorganisms are not limited to a particular genus, species, strain, or serotype. The microorganisms can be identified by determining the nucleic acid sequence and/or sequence variations in a target microorganism sequence relative to one or more reference sequences. The reference sequence(s) can be obtained from, for example, other microorganisms from the same or different genus, species strain or serotype, or from a host prokaryotic or eukaryotic organism.

Identification and typing of bacterial pathogens can be critical in the clinical management of infectious diseases. Precise identity of a microbe is used not only to differentiate a disease state from a healthy state, but also is fundamental to determining whether and which antibiotics or other antimicrobial therapies are most suitable for treatment. Traditional methods of pathogen typing have used a variety of phenotypic features, including growth characteristics, color, cell or colony morphology, antibiotic susceptibility, staining, smell and reactivity with specific antibodies to identify bacteria. All of these methods require culture of the suspected pathogen, which suffers from a number of serious shortcomings, including high material and labor costs, danger of worker exposure, false positives due to mishandling and false negatives due to low numbers of viable cells or due to the fastidious culture requirements of many pathogens. In addition, culture methods require a relatively long time to achieve diagnosis, and because of the potentially life-threatening nature of such infections, antimicrobial therapy is often started before the results can be obtained.

In many cases, the pathogens are very similar to the organisms that make up the normal flora, and can be indistinguishable from the innocuous strains by the phenotypic methods cited above. In these cases, determination of the presence of the pathogenic strain can require the higher resolution afforded by the fragmentation and hybridization-based methods provided herein. For example, PCR amplification of a target nucleic acid sequence followed by fragmentation and hybridization-based sequencing using matrix-assisted laser desorption/ionization time-of-flight mass spectrometry, followed by screening for sequence variations as provided herein, allows reliable discrimination of sequences differing by only one nucleotide and combines the discriminatory power of the sequence information generated with the speed of MALDI-TOF MS. Similarly, methods for identifying a portion of a target nucleic acid by comparing one or more mass peaks or mass peak patterns can be used to detect such sequence variations.

For example, bacteria typing using more reliable longer sequence regions, such as the full-length 16S rRNA gene, can be accomplished using the fragmentation and hybridization-based sequencing methods provided herein, including fragmentation-based sequencing methods in a comparative format. To illustrate, the sequence of one or more known bacteria type(s) can be obtained and compared to the sequence of an unknown bacteria type.

7. Molecular Breeding and Directed Evolution

In one embodiment, the methods disclosed herein can be used to determine the sequence or portion of a target nucleic acid when the target nucleic acid can represent a nucleic acid, virus, or organism, that has been modified. Such methods can be used correlate the properties of a biomolecule or the phenotype of an organism or virus with the genotype of the biomolecule, organism or virus. For example, the methods disclosed herein can be used to identify a nucleotide sequence, mass peak or mass peak pattern, as associated with a particular property of a target nucleic acid, a protein encoded by the target nucleic acid, or a virus or organism containing the target nucleic acid.

For example, the methods herein can be used to identify particular protein properties as associated with a target nucleic acid sequence, mass peak or mass peak pattern. In this example, one or more proteins can be redesigned by modifying the one or more genes encoding the proteins using any of a variety of methods known in the art for gene modification, including DNA shuffling (U.S. Pat. Nos. 6,117,679 and 6,537,746), error-prone PCR (Caldwell, R. C. and Joyce, G. F. (1992) PCR Methods and Applications 2:28-33), cassette mutagenesis (Goldman, E R and Youvan D C (1992) Bio/Technology 10: 1557-1561; Delagrave et al. Protein Engineering 6:327-331 (1993)), and random codon mutagenic methods (U.S. Pat. Nos. 5,264,563 and 5,723,323). Sequences or portions of genes encoding redesigned proteins with one or more particular properties can be examined using the methods disclosed herein, and one or more mass peaks can be identified as being associated with the one or more particular properties of the redesigned proteins. Exemplary protein properties include binding ability, catalytic ability, thermal stability, sensitivity to proteases, expression level, solubility, membrane insertion or association, post-translational modifications, optical properties, electron transfer properties, organelle targeting, ability to be secreted, susceptibility to degradation in the liver, immunogenicity, and ability to be transported across biological barriers including absorption from the gut into the bloodstream and crossing the blood brain barrier.

Methods to identify one or more mass peaks as being associated with the one or more particular properties of the redesigned proteins include analysis of the pattern of mass peaks for the genes encoding one or more redesigned proteins possessing the one or more particular properties, and identifying a nucleotide sequence or one or more mass peaks or mass peak characteristics that are associated with those particular properties. Determining sequences or mass peaks associated with particular properties can be accomplished by determining sequences or mass peaks common to two or more genes encoding proteins with particular properties, and typically the sequences or mass peaks is/are common to at least 50%, at least 70%, at least 85%, at least 90%, or at least 95% of genes encoding the proteins with particular properties. Determining sequences or mass peaks associated with particular properties also can be accomplished, even if only one such protein possesses the particular properties, by determining sequences or mass peaks unique to the gene encoding that protein.

In accord with the method above, another embodiment includes a method for identifying one or more genes encoding a protein having one or more particular properties, where the method includes fragmenting a gene, hybridizing the gene fragments to one or more capture oligonucleotide probes, where two or more gene fragments have different nucleotide sequences that hybridize to capture oligonucleotide probes that have the same nucleotide sequence, and measuring the mass of the two or more gene fragments. In one embodiment, upon measuring the mass peaks, one or more of the measured mass peaks can be compared to one or more reference mass peaks, where the one or more reference mass peaks are associated with the one or more particular properties of the redesigned proteins. Reference mass peaks can be experimentally determined using, for example, the methods discussed hereinabove, or can be theoretically determined. In another embodiment, the nucleotide sequence of the target nucleic acid can be constructed and a target nucleic acid that contains a sequence associated with one or more particular protein properties can be identified as a gene that encodes a protein with such properties.

Further in accordance with the present embodiment, one or more mass peaks associated with the one or more particular properties of redesigned protein can be further analyzed using the methods described herein to provide nucleotide sequence information regarding the target nucleic acid gene encoding the redesigned protein. For example, target nucleic acid sequence information can be obtained by comparing one or more mass peak characteristics with one or more reference mass peak characteristics where the one or more reference mass peak characteristics correspond to a particular nucleotide sequence at one or more nucleotide positions on the target nucleic acid. In another example, the nucleotide sequence of one or more target nucleic acid fragments can be determined according to measured mass peak characteristics or by using the sequence construction methods provided herein. In yet another example, the entire target nucleic acid sequence, or portions thereof can be determined using the sequence construction methods provided herein.

In another example, one or more viruses can be redesigned by modifying the viral genome using any of a variety of methods including viral genome shuffling (U.S. Pat. No. 6,596,539), and viral mutation and selection methods. The modified viral genome that results in one or more viruses with one or more particular properties can be examined using the methods disclosed herein, and one or more mass peaks can be identified as being associated with the one or more particular properties of the modified viruses. Exemplary viral properties include viral infectivity, replication, host range, tropism, gene function, transcriptional regulatory sequence function, capability to replicate in a non-permissive cell, host range and/or cell tropism, virus titer (e.g., virulence), pathogenicity or capacity to produce disease, infectivity, packaging capacity, physical/chemical stability of viral particles, intracellular stability, expression of one or more viral genes, chromosomal integration, tissue specificity and capability to infect preferentially specific organs, immunogenicity or virus or viral protein in a host (e.g., a human), function as a biological adjuvant (e.g., to co-express a viral-encoded human cytokine), and function as a therapeutic (e.g., capacity to induce a general antiviral host response—such as interferon production).

Methods to identify one or more mass peaks as being associated with the one or more particular properties of the redesigned viruses include analysis of the pattern of mass peaks for the viral sequences of one or more redesigned viruses possessing the one or more particular properties, and identifying a nucleotide sequence or one or more mass peaks or mass peak characteristics that are associated with those particular properties. Determining sequences or mass peaks associated with particular properties can be accomplished by determining sequences or mass peaks common to two or more viral sequences with particular properties, and typically the sequences or mass peaks is/are common to at least 50%, at least 70%, at least 85%, at least 90%, or at least 95% of viral sequences with particular properties. Determining sequences or mass peaks associated with particular properties also can be accomplished, even if only one such virus possesses the particular properties, by determining sequences or mass peaks unique to the viral sequence.

In accord with the method above, another embodiment includes a method for identifying one or more viral sequences having one or more particular properties, where the method includes fragmenting a viral nucleic acid, hybridizing the viral nucleic acid fragments to one or more capture oligonucleotide probes, where two or more viral nucleic acid fragments have different nucleotide sequences that hybridize to capture oligonucleotide probes that have the same nucleotide sequence, and measuring the mass of the two or more viral nucleic acid fragments. In one embodiment, upon measuring the mass peaks, one or more of the measured mass peaks can be compared to one or more reference mass peaks, where the one or more reference mass peaks are associated with the one or more particular properties of the redesigned viruses. Reference mass peaks can be experimentally determined using, for example, the methods discussed hereinabove, or can be theoretically determined. In another embodiment, the nucleotide sequence of the viral nucleic acid can be constructed and a viral nucleic acid that contains a sequence associated with one or more particular protein properties can identify a viral sequence that encodes a protein with such properties.

Further in accordance with the present embodiment, one or more mass peaks associated with the one or more particular properties of redesigned virus can be further analyzed using the methods described herein to provide nucleotide sequence information regarding the viral nucleic acid of the redesigned virus. For example, viral nucleic acid sequence information can be obtained by comparing one or more mass peak characteristics with one or more reference mass peak characteristics where the one or more reference mass peak characteristics correspond to a particular nucleotide sequence at one or more nucleotide positions on the viral nucleic acid. In another example, the nucleotide sequence of one or more viral nucleic acid fragments can be determined according to measured mass peak characteristics or by using the sequence construction methods provided herein. In yet another example, the entire viral nucleic acid sequence, or portions thereof can be determined using the sequence construction methods provided herein.

Further contemplated herein are methods to identify one or more mass peaks as being associated with the one or more particular properties of organisms, such as genetically modified organisms. Exemplary organisms include plants such as agricultural plants including corn, rice, wheat, rye, oats, barley, pea, beans, lentil, peanut, yam bean, cowpeas, velvet beans, soybean, clover, alfalfa, lupine, vetch, lotus, sweet clover, wisteria, sweetpea, sorghum, millet, sunflower, and canola; birds including turkey and chicken; fish; insects; nematodes; non-human mammals including livestock such as a pig, cow, horse and other livestock. Methods for modifying the genomes of various organisms are known in the art, and include DNA shuffling (U.S. Pat. Nos. 6,379,964 and 6,500,617), and also include traditional breeding by sexual reproduction. Properties of the organism can vary according to the organism, but generally include viability, resistance to disease, growth rate, reproduction abilities, nutritional requirements, water requirements, temperature sensitivity, and resistance to environmental stresses. Methods to identify one or more mass peaks as being associated with the one or more particular properties of organisms, such as genetically modified organisms can be carried out using the methods hereinabove described with regard to viruses.

8. Target Nucleic Acid Fragments as Markers

In other embodiments, target nucleic acid fragments can be used as markers or indicators of sequences or portions of a large target nucleic acid. Such embodiments do not require determination of the entire sequence of the target nucleic acid, but can include determining the sequence of portions of the target nucleic acid, or simply determining the mass peak pattern of target nucleic acid fragments. These embodiments also do not require that the target nucleic acid fragments be overlapping; thus, for these embodiments, target nucleic acid fragments can be overlapping or non-overlapping. Such methods can include, for example, fingerprinting and fingerprinting related methods and other methods that include use of non-overlapping DNA fragments as indicators of sequences or portions of a target nucleic acid. Fingerprinting methods that use amplification steps such as amplified ribosomal DNA restriction analysis (ARDRA), random amplified polymorphic DNA analysis (RAPD), and amplified fragment length polymorphism (AFLP), can be used in the methods disclosed herein.

In one embodiment, fragments of a target nucleic acid can be formed, hybridized to an array of capture nucleic acids, and the mass of the fragments determined, to create a pattern of mass peaks characterized by one, two, three, or more characteristics such as the position of the capture oligonucleotide probe with which the target nucleic acid hybridizes, the mass, and the signal to noise ratio of the mass peak. Such a pattern of mass peaks can be used as an indicator of the sequence or portion of a target nucleic acid.

In one embodiment, specifically designed primers and amplification methods can control amplification in such a way that only a subset of target nucleic acid fragments is amplified, and this subset of fragments can then be hybridized to an array of capture oligonucleotide probes and mass analyzed. This embodiment can use as a target nucleic acid: a gene, a chromosome fragment, yeast artificial chromosome (YAC), bacterial artificial chromosome (BAC), an entire chromosome, an entire genome or any other suitable nucleic acid molecule; or a plurality of genes, chromosome fragments, YACs, BACs, entire chromosomes and entire genomes, from one or more different organisms such as a population of a species or strains. Methods for amplifying subsets of nucleic acid fragments are known in the art, such as amplified fragment length polymorphism (AFLP) methods (see, e.g., U.S. Pat. No. 6,045,994).

In accordance with this embodiment, one or more restriction enzymes are used to create fragments of the target nucleic acid. Typically, two restriction enzymes that cleave at different nucleotide sequences are used. For example, a rare cutter (a restriction enzyme that recognizes a long nucleotide sequence such as 6 nucleotides, and thus, cuts at fewer sites on a nucleic acid) and a common cutter (restriction enzyme that recognizes a short nucleotide sequence such as 4 nucleotides, and thus, cuts at more sites on a nucleic acid) can be used. In other examples, two rare cutters or two common cutters can be used. The choice of the number of restriction enzymes and the specificity of the enzymes can be made according to the length of the target nucleic acid and the desired number and length of target nucleic acid fragments.

PCR amplification of restriction fragments can be carried out regardless of whether or not the nucleotidic sequence of the ends of the restriction fragments is known. This can be achieved by first ligating synthetic oligonucleotides (adaptors) of known sequence to both ends of the restriction fragments, thus providing each restriction fragment with two common tags that can be complementary to the primers used in PCR amplification.

Typically, restriction enzymes produce either blunt ends, in which the terminal nucleotides of both strands are base paired, or “sticky” ends in which one of the two strands protrudes to give a short single-stranded region. In the case of restriction fragments with blunt ends, adaptors are ligated to one strand of the blunt end. In the case of restriction fragments with sticky ends, the adaptors have a region that is complementary to the single-stranded region of the restriction fragment. Such an adaptor is first hybridized to the complementary portion of the single-stranded region of the restriction fragment in such a way that the adaptor end is adjacent to the end of one strand of the restriction fragment; then the adaptor is ligated to the adjacent restriction fragment end.

Consequently, for each type of restriction cleavage, different adaptors can be designed so as to permit one end of the adaptor to be ligated to a particular corresponding restriction fragment. Typically, the adaptors are approximately 10 to 30 nucleotides long, and typically 12 to 22 nucleotides long. Using a ligase enzyme, the adaptors are ligated to the mixture of restriction fragments. When using a large molar excess of adaptors relative to restriction fragments, nearly all restriction fragments are ligated to adaptors at both ends. Restriction fragments prepared with this method are referred to as “tagged restriction fragments.”

Each tagged restriction fragment has the following general structure: a variable DNA sequence flanked by constant DNA sequences at each end of the tagged restriction fragment. The constant DNA sequence contains part or all of the recognition sequence of the restriction endonuclease and also contains the sequence of the adaptor attached to each end of the tagged restriction fragment. The variable sequences of the restriction fragments are located between the constant DNA sequences, and thus include the portion of the restriction fragment that does not contain the restriction endonuclease recognition sequences. The variable sequences can be known or unknown, and typically vary between restriction fragments. Consequently, the nucleotide sequences flanking the constant DNA sequences can be a large mixture of different sequences.

In one embodiment, the adaptors can be exact complements to PCR primers. For example, the restriction fragment can carry the same adaptor at both of its ends and a single PCR primer can hybridize to the adaptors without hybridizing to any part of the restriction fragment sequence, and can be used to amplify the restriction fragment. In another example, using, for example, two different restriction enzymes to cleave the DNA, two different adaptors can be ligated to the ends of the restriction fragments. In this case, one or two different PCR primers can be used to amplify such restriction fragments. In this embodiment, the PCR primers are used to amplify all tagged restriction fragments, without regard to the variable sequences of the restriction fragments.

Regardless of whether or not the tagged restriction fragments are amplified in the above step, the tagged restriction fragments are then amplified using variable sequence-specific PCR primers which contain a first nucleotide sequence portion and a second sequence portion. The first sequence portion is designed to perfectly base pair with the constant DNA sequence of the tagged restriction fragment. The second sequence portion can contain any selected sequence or a random sequence, and ranges in length from 1 to about 10 nucleotides. The second sequence portion hybridizes to only a subset of the tagged restriction fragments, resulting in only the hybridized subset of tagged restriction fragments being amplified. In one embodiment, several different sequence-specific PCR primers can be used that have different sequences in their second sequence portions, in order to amplify a larger subset of tagged restriction fragments.

The addition of the second sequence portions to the 3′ end of the sequence-specific primers determines which tagged restriction fragments are amplified in the PCR step: the sequence-specific primers will only initiate DNA synthesis on those tagged restriction fragments in which the second portions of the sequence-specific PCR primers can base pair with the tagged restriction fragments.

After sequence specific amplification of a subset of the tagged restriction fragments, the restriction fragments (which also can be referred to as target nucleic acid fragments) can be, if desired, further fragmented according to the methods disclosed herein. For example, the target nucleic acid fragments (restriction fragments) can be subjected to additional sequence-specific cleavage, base-specific cleavage, or non-specific cleavage. The target nucleic acid fragments are then hybridized to an array of capture oligonucleotide probes. After hybridization, the target nucleic acid fragments can be, if desired, further fragmented according to the methods disclosed herein. For example, the target nucleic acid fragments can be subjected to base-specific cleavage. Cleavage prior to hybridization or after hybridization can be carried out, for example, to achieve a desired level of complexity of the target nucleic acid fragments hybridized to one or more capture oligonucleotide probes, or to achieve the desired length of target nucleic acid fragment, for example, for desired accuracy of mass determination using mass spectroscopy.

9. Detecting the Presence of Viral or Bacterial Nucleic Acid Sequences Indicative of an Infection

The methods provided herein can be used to determine the presence of viral or bacterial nucleic acid sequences indicative of an infection by identifying sequence variations that are present in the viral or bacterial nucleic acid sequences relative to one or more reference sequences. The reference sequence(s) can include, but are not limited to, sequences obtained from related non-infectious organisms, or sequences from host organisms.

Viruses, bacteria, fungi and other infectious organisms contain distinct nucleic acid sequences, including polymorphisms, which are different from the sequences contained in the host cell. A target DNA sequence can be part of a foreign genetic sequence such as the genome of an invading microorganism, including, for example, bacteria and their phages, viruses, fungi and protozoa. The processes provided herein are particularly applicable for distinguishing between different variants or strains of a microorganism in order, for example, to choose an appropriate therapeutic intervention. Examples of disease-causing viruses that infect humans and animals and that can be detected by a disclosed process include but are not limited to Retroviridae (e.g., human immunodeficiency viruses such as HIV-1 (also referred to as HTLV-III, LAV or HTLV-III/LAV; Ratner et al., Nature 313:227-284 (1985); Wain Hobson et al., Cell 40:9-17 (1985), HIV-2 (Guyader et al., Nature, 328:662-669 (1987); European Patent Publication No. 0 269 520; Chakrabarti et al., Nature 328:543-547 (1987); European Patent Application No. 0 655 501), and other isolates such as HIV-LP (International Publication No. WO 94/00562); Picornaviridae (e.g., polioviruses, hepatitis A virus, (Gust et al., Intervirology, 20:1-7 (1983)); enteroviruses, human coxsackie viruses, rhinoviruses, echoviruses); Calcivirdae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arenaviridae (hemorrhagic fever viruses); Reoviridae (e.g., reoviruses, orbiviruses and rotaviruses); Birnaviridae; Hepadnaviridae (Hepatitis B virus); Parvoviridae (parvoviruses); Papovaviridae; Hepadnaviridae (Hepatitis B virus); Parvoviridae (most adenoviruses); Papovaviridae (papilloma viruses, polyoma viruses); Adenoviridae (most adenoviruses); Herpesviridae (herpes simplex virus type 1 (HSV-1) and HSV-2, varicella zoster virus, cytomegalovirus, herpes viruses; Poxyiridae (variola viruses, vaccinia viruses, pox viruses); Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted, i.e., Hepatitis C); Norwalk and related viruses, and astroviruses.

Examples of infectious bacteria include but are not limited to Helicobacter pyloris, Borelia burgdorferi, Legionella pneumophilia, Mycobacteria sp. (e.g., M. tuberculosis, M avium, M. intracellulare, M. kansaii, M. gordonae), Staphylococcus aureus, Neisseria gonorrheae, Neisseria meningitidis, Listeria monocytogenes, Streptococcus pyogenes (Group A Streptococcus), Streptococcus agalactiae (Group B Streptococcus), Streptococcus sp. (viridans group), Streptococcus faecalis, Streptococcus bovis, Streptococcus sp. (anaerobic species), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringens, Clostridium tetani. Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces israelli.

Examples of infectious fungi include but are not limited to Cryptococcus neoformans, Histoplasma capsulatum, Coccidioides immitis, Blastomyces dermatitidis, Chlamydia trachomatis, Candida albicans. Other infectious organisms include protists such as Plasmodium falciparum and Toxoplasma gondii.

10. Antibiotic Profiling

Mass analysis of target nucleic acid fragments as provided herein can improve the speed and accuracy of detection of nucleotide changes involved in drug resistance, including antibiotic resistance. Genetic loci involved in resistance to isoniazid, rifampin, streptomycin, fluoroquinolones, and ethionamide have been identified [Heym et al., Lancet 344:293 (1994) and Morris et al., J. Infect. Dis. 171:954 (1995)]. A combination of isoniazid (inh) and rifampin (rif) along with pyrazinamide and ethambutol or streptomycin, is routinely used as the first line of attack against confirmed cases of M. tuberculosis [Banerjee et al., Science 263:227 (1994)]. The increasing incidence of such resistant strains necessitates the development of rapid assays to detect them and thereby reduce the expense and community health hazards of pursuing ineffective, and possibly detrimental, treatments. The identification of some of the genetic loci involved in drug resistance has facilitated the adoption of mutation detection technologies for rapid screening of nucleotide changes that result in drug resistance.

11. Identifying Disease Markers

Provided herein are methods for the rapid and accurate identification of sequence variations that are genetic markers of disease, which can be used to diagnose or determine the prognosis of a disease. Diseases characterized by genetic markers can include, but are not limited to, atherosclerosis, obesity, diabetes, autoimmune disorders, and cancer. Diseases in all organisms have a genetic component, whether inherited or resulting from the body's response to environmental stresses, such as viruses and toxins. The ultimate goal of ongoing genomic research is to use this information to develop ways to identify, treat and potentially cure these diseases. The first step has been to screen disease tissue and identify genomic changes at the level of individual samples. The identification of these “disease” markers is dependent on the ability to detect changes in genomic markers in order to identify errant genes or polymorphisms. Genomic markers (all genetic loci including single nucleotide polymorphisms (SNPs), microsatellites and other noncoding genomic regions, tandem repeats, introns and exons) can be used for the identification of all organisms, including humans. These markers provide a way to not only identify populations but also allow stratification of populations according to their response to disease, drug treatment, resistance to environmental agents, and other factors.

12. Haplotyping

The methods provided herein can be used to detect haplotypes. In any diploid cell, there are two haplotypes at any gene or other chromosomal segment that contain at least one distinguishing variance. In many well-studied genetic systems, haplotypes are more powerfully correlated with phenotypes than single nucleotide variations. Thus, the determination of haplotypes is valuable for understanding the genetic basis of a variety of phenotypes including disease predisposition or susceptibility, response to therapeutic interventions, and other phenotypes of interest in medicine, animal husbandry, and agriculture.

Haplotyping procedures as provided herein permit the selection of a portion of sequence from one of an individual's two homologous chromosomes and to genotype linked SNPs on that portion of sequence. The direct resolution of haplotypes can yield increased information content, improving the diagnosis of any linked disease genes or identifying linkages associated with those diseases.

13. DNA Repeats

The fragmentation-based methods provided herein allow for rapid detection of sequence variations in DNA repeats. Various DNA repeats can be associated with disease (Thangavelu et al., Prenat. Diagn. 18:922-25 (1998); Bennett et al., J. Autoimmun. 9:415-21 (1996)). DNA repeats include satellites, minisatellites and microsatellites. Satellites can range in unit size from 2-base unit repeats to about 1000-base unit repeats, or more, and, typically the repeat units are present in a range of about 1000 repeats to about 10,000 repeats. Minisatellites, also termed short tandem repeats (or STRs) can range in unit size from 3-base unit repeats to about 100-base unit repeats, and, typically the repeat units are present in a range of about 2 repeats to about 100 repeats, or more, such that the minimum length of a minisatellite is typically about 500 bases. Microsatellites can range in unit size from 1-base unit repeats to about 7-base unit repeats, and, typically the repeat units are present in a range of about 5 repeats to about 100 repeats. Microsatellites can be located close to genes on a chromosome and can play a role in gene expression. Detection of variations in satellites, minisatellites or microsatellites can be used as a marker of variants or tendency toward disease.

Microsatellites (sometimes referred to as variable number of tandem repeats or VNTRs) are short tandemly repeated nucleotide units of one to seven or more bases, the most prominent among them being di-, tri-, and tetranucleotide repeats. Microsatellites are present every 100,000 bp in genomic DNA (J. L. Weber and P. E. Can, Am. J. Hum. Genet. 44:388 (1989); J. Weissenbach et al., Nature 359:794 (1992)). CA dinucleotide repeats, for example, make up about 0.5% of the human extra-mitochondrial genome; CT and AG repeats together make up about 0.2%. CG repeats are rare, most probably due to the regulatory function of CpG islands. Microsatellites are highly polymorphic with respect to length and widely distributed over the whole genome with a main abundance in non-coding sequences, and their function within the genome is unknown.

Microsatellites are important in forensic applications, as a population maintains a variety of microsatellites characteristic for that population and distinct from other populations, which do not interbreed.

Many changes within microsatellites can be silent, but some can lead to significant alterations in gene products or expression levels. For example, trinucleotide repeats found in the coding regions of genes are affected in some tumors (C. T. Caskey et al., Science 256:784 (1992) and alteration of the microsatellites can result in a genetic instability that results in a predisposition to cancer (P. J. McKinnen, Hum. Genet. 1(75):197 (1987); J. German et al., Clin. Genet. 35:57 (1989)).

The methods provided herein also can be used to identify minisatellites or short tandem repeats (STRs) in some target sequences of the a genome relative to, for example, reference genomic sequences of a genome that does not contain STR regions. STR regions are polymorphic regions that are not related to any disease or condition. Many loci in the human genome contain a polymorphic short tandem repeat (STR) region. STR loci contain short, repetitive sequence elements of 3 to 100 base pairs in length. It is estimated that there are 200,000 expected trimeric and tetrameric STRs, which are present as frequently as once every 15 kb in the human genome (see, e.g., International PCT application No. WO 9213969 A1, Edwards et al., Nucl. Acids Res. 19:4791 (1991); Beckmann et al. Genomics 12:627-631 (1992)). Nearly half of these STR loci are polymorphic, providing a rich source of genetic markers. Variation in the number of repeat units at a particular locus is responsible for the observed polymorphism reminiscent of variable nucleotide tandem repeat (VNTR) loci (Nakamura et al. Science 235:1616-1622 (1987)); and minisatellite loci (Jeffreys et al. Nature 314:67-73 (1985)), which contain longer repeat units, and microsatellite or dinucleotide repeat loci (Luty et al. Nucleic Acids Res. 19:4308 (1991); Litt et al. Nucleic Acids Res. 18:4301 (1990); Litt et al. Nucleic Acids Res. 18:5921 (1990); Luty et al. Am. J. Hum. Genet. 46:776-783 (1990); Tautz Nucl. Acids Res. 17:6463-6471 (1989); Weber et al. Am. J. Hum. Genet. 44:388-396 (1989); Beckmann et al. Genomics 12:627-631 (1992)).

Examples of STR loci include, but are not limited to, pentanucleotide repeats in the human CD4 locus (Edwards et al., Nucl. Acids Res. 19:4791 (1991)); tetranucleotide repeats in the human aromatase cytochrome P-450 gene (CYP19; Polymeropoulos et al., Nucl. Acids Res. 19:195 (1991)); tetranucleotide repeats in the human coagulation factor XIII A subunit gene (F13A1; Polymeropoulos et al., Nucl. Acids Res. 19:4306 (1991)); tetranucleotide repeats in the F13B locus (Nishimura et al., Nucl. Acids Res. 20:1167 (1992)); tetranucleotide repeats in the human c-les/fps, proto-oncogene (FES; Polymeropoulos et al., Nucl. Acids Res. 19:4018 (1991)); tetranucleotide repeats in the LFL gene (Zuliani et al., Nucl. Acids Res. 18:4958 (1990)); trinucleotide repeats polymorphism at the human pancreatic phospholipase A-2 gene (PLA2; Polymeropoulos et al., Nucl. Acids Res. 18:7468 (1990)); tetranucleotide repeats polymorphism in the VWF gene (Ploos et al., Nucl. Acids Res. 18:4957 (1990)); and tetranucleotide repeats in the human thyroid peroxidase (hTPO) locus (Anker et al., Hum. Mol. Genet. 1:137 (1992)).

14. Detecting Allelic Variation

The methods provided herein allow for high-throughput, fast and accurate detection of allelic variants. Studies of allelic variation involve not only detection of a specific sequence in a complex background, but also the discrimination between sequences with few, or single, nucleotide differences. One method for the detection of allele-specific variants by PCR is based upon the fact that it is difficult for Taq polymerase to synthesize a DNA strand when there is a mismatch between the template strand and the 3′ end of the primer. An allele-specific variant can be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect (Kwok et al., Nucl. Acids Res. 18:999 [1990]).) The fragmentation and hybridization-based methods provided herein overcome the limitations of the primer extension method.

15. Determining Allelic Frequency

The methods herein described are useful for identifying one or more genetic markers whose frequency changes within the population as a function of age, ethnic group, sex or some other criteria. For example, the age-dependent distribution of ApoE genotypes is known in the art (see, Schachter et al. Nature Genetics 6:29-32 (1994)). The frequencies of polymorphisms known to be associated at some level with disease also can be used to detect or monitor progression of a disease state. For example, the N291S polymorphism (N291S) of the Lipoprotein Lipase gene, which results in a substitution of a serine for an asparagine at amino acid codon 291, leads to reduced levels of high density lipoprotein cholesterol (HDL-C) that is associated with an increased risk of males for arteriosclerosis and in particular myocardial infarction (see, Reymer et al. Nature Genetics 10:28-34 (1995)). In addition, determining changes in allelic frequency can allow the identification of previously unknown polymorphisms and ultimately a gene or pathway involved in the onset and progression of disease.

16. Epigenetics

The methods provided herein can be used to study variations in a target nucleic acid or protein, relative to a reference nucleic acid, that are not based on sequence, e.g., the identity of bases that are the naturally occurring monomeric units of the nucleic acid. For example, the specific cleavage reagents employed in the methods provided herein can recognize differences in sequence-independent features such as methylation patterns, the presence of modified bases, or differences in higher order structure between the target molecule and the reference molecule, to generate fragments that are cleaved at sequence-independent sites. Epigenetics is the study of the inheritance of information based on differences in gene expression rather than differences in gene sequence. Epigenetic changes refer to mitotically and/or meiotically heritable changes in gene function or changes in higher order nucleic acid structure that cannot be explained by changes in nucleic acid sequence. Examples of features that are subject to epigenetic variation or change include, but are not limited to, DNA methylation patterns in animals, histone modification and the Polycomb-trithorax group (Pc-G/tx) protein complexes (see, e.g., Bird, A., Genes Dev., 16:6-21 (2002)).

Epigenetic changes usually, although not necessarily, lead to changes in gene expression that are usually, although not necessarily, inheritable. For example, as discussed above, changes in methylation patterns is an early event in cancer and other disease development and progression. In many cancers, certain genes are inappropriately switched off or switched on due to aberrant methylation. The ability of methylation patterns to repress or activate transcription can be inherited. The Pc-G/trx protein complexes, like methylation, can repress transcription in a heritable fashion. The Pc-G/trx multiprotein assembly is targeted to specific regions of the genome where it effectively freezes the embryonic gene expression status of a gene, whether the gene is active or inactive, and propagates that state stably through development. The ability of the Pc-G/trx group of proteins to target and bind to a genome affects only the level of expression of the genes contained in the genome, and not the properties of the gene products. The methods provided herein can be used with specific cleavage reagents that identify variations in a target sequence relative to a reference sequence that are based on sequence-independent changes, such as epigenetic changes.

### EXAMPLE 1

To reconstruct the underlying DNA sequence, one can use the methods described and exemplified in this example to use techniques for nucleotide sequence analysis of Sequencing By Hybridization as well as techniques for nucleotide sequence analysis by Mass Spectrometry. In particular, one can transform the experimental data into a subgraph of a de Bruijn graph, see Pevzner, J. Biomol. Struct. Dyn., 7:63-73 (1989). One can then search for Eulerian paths in this graph, where cycles and bulges have to be broken in advance, see Pevzner et al., Proc. Natl. Acad. Sci. USA 98:9748-9753 (2001).

As an example, let ACATGAGCTTACAAC (SEQ ID NO: 1) be the DNA sequence under consideration. The cleavage reaction unspecifically cleaves this DNA (or RNA) molecule into fragments of 5-7 nt. Finally, the resulting fragments are bound to a hybridization chip containing 16 positions with 4 degenerate bases, each degenerate base binding either purines (letter R, A or G) or pyrimidines (letter Y, C or T). In this degenerate alphabet, the sequence under consideration becomes RYRYRRRYYYRYRRY. Then, the following binding pattern occurs on the chip:

| De- | |
|---|---|
| generate | |
| pattern | Fragments attaching to hybridization spot |
| RRRR | (no fragments) |
| RRRY | CATGAGC, ATGAGC, ATGAGCT, TGAGC, TGAGCT, |
| | GAGCTT, GAGCT, GAGCTT, GAGCTTA |
| RRYR | (no fragments) |
| RRYY | ATGAGCT, TGAGCT, TGAGCTT, GAGCT, GAGCTT, |
| | GAGCTTA, AGCTT, AGCTTA, AGCTTAC |
| RYRR | ACATGA, ACATGAG, CATGA, CATGAG, CATGAGC, |
| | ATGAG, ATGAGC, ATGAGCT, CTTACAA, TTACAA, |
| | TTACAAC |
| RYRY | ACATG, ACATGA, ACATGAG |
| RYYR | (no fragments) |
| RYYY | TGAGCTT, GAGCTT, GAGCTTA, AGCTT, AGCTTA, |
| | AGCTTAC, GCTTA, GCTTAC, GCTTACA |
| YRRR | ACATGAG, CATGAG, CATGAGC, ATGAG, ATGAGC, |
| | ATGAGCT, TGAGC, TGAGCT, TGAGCTT |
| YRRY | TTACAAC |
| YRYR | ACATG, ACATGA, ACATGAG, CATGA, CATGAG, |
| | CATGAGC, GCTTACA, CTTACA, CTTACAA, TTACA, |
| | TTACAA, TTACAAC |
| YRYY | (no fragments) |
| YYRR | (no fragments) |
| YYRY | AGCTTAC, GCTTAC, GCTTACA, CTTAC, CTTACA, |
| | CTTACAA, TTACA, TTACAA, TTACAAC |
| YYYR | GAGCTTA, AGCTTA, AGCTTAC, GCTTA, GCTTAC, |
| | GCTTACA, CTTAC, CTTACA, CTTACAA |
| YYYY | (no fragments) |

Using mass spectrometry analysis, the composition of a fragment can be determined, see for example Bocker, Lect. Notes Comp. Sci. 2812:476-487 (2003). Then mass spectra corresponding to the following compomers are measured:

| Degenerate | |
|---|---|
| pattern | Compomers detected on hybridization spot |
| RRRR | (no peaks) |
| RRRY | A₂C₂G₂T₁, A₂C₁G₂T₁, A₂C₁G₂T₂, A₁C₁G₂T₁, A₁C₁G₂T₂, |
| | A₁C₁G₂T₃, A₁C₁G₂T₁, A₁C₁G₂T₂, A₂C₁G₂T₁ |
| RRYR | (no peaks) |
| RRYY | A₂C₁G₂T₂, A₁C₁G₂T₂, A₁C₁G₂T₃, A₁C₁G₂T₁, A₁C₁G₂T₂, |
| | A₂C₁G₂T₂, A₁C₁G₁T₂, A₂C₁G₁T₂, A₂C₂G₁T₂ |
| RYRR | A₃C₁G₁T₁, A₃C₁G₂T₁, A₂C₁G₁T₁, A₂C₁G₂T₁ (twice), |
| | A₂C₂G₂T₁, A₂G₂T₁, A₂C₁G₂T₁, A₂C₁G₂T₂, |
| | A₃C₂T₂ (twice), A₃C₁T₂ |
| RYRY | A₂C₁G₁T₁, A₃C₁G₁T₁, A₃C₁G₂T₁ |
| RYYR | (no peaks) |
| RYYY | A₁C₁G₂T₃, A₁C₁G₂T₂, A₂C₁G₂T₂, A₁C₁G₁T₂ (twice), |
| | A₂C₁G₁T₂, A₂C₂G₁T₂ (twice), A₁C₂G₁T₂ |
| YRRR | A₃C₁G₂T₁, A₂C₁G₂T₁ (twice), A₂C₂G₂T₁, A₂G₂T₁, |
| | A₂C₁G₂T₂, A₁C₁G₂T₁, A₁C₁G₂T₂, A₁C₁G₂T₃ |
| YRRY | A₃C₂T₂ |
| YRYR | A₂C₁G₁T₁ (twice), A₃C₁G₁T₁, A₃C₁G₂T₁, A₂C₁G₂T₁, |
| | A₂C₂G₂T₁, A₂C₂G₁T₂, A₂C₂T₂, A₃C₂T₂ (twice), |
| | A₂C₁T₂, A₃C₁T₂ |
| YRYY | (no peaks) |
| YYRR | (no peaks) |
| YYRY | A₂C₂G₁T₂ (twice), A₁C₁G₁T₂, A₁C₂T₂, A₂C₂T₂, |
| | A₃C₂T₂ (twice), A₂C₁T₂, A₃C₁T₂ |
| YYYR | A₂C₁G₂T₂, A₂C₁G₁T₂, A₂C₂G₁T₂ (twice), A₁C₁G₁T₂, |
| | A₁C₂G₁T₂, A₁C₂T₂, A₂C₂T₂, A₃C₂T₂ |
| YYYY | (no peaks) |

This information is used in a branch-and-bound search as follows: Suppose that ACATGAG is a known prefix of the correct sequence. The identity of the next base can be randomly assigned, and then compared to one or more mass spectra. Assigning the next base is an A, then peaks for the following fragments and compomers in several different mass spectra are predicted:

| Fragment: | Compomer: | Spectra corresponding to: |
|---|---|---|
| CATGAGA | A₃C₁G₂T₁ | YRYR, RYRR, YRRR, RRRR |
| ATGAGA | A₃G₂T₁ | RYRR, YRRR, RRRR |
| TGAGA | A₂G₂T₁ | YRRR, RRRR |

The mass spectra contradict this hypothesis: If ACATGAGA was the correct nucleotide at this locus, then the mass spectrum corresponding to hybridization position RRRR would contain at least three peaks. But not a single peak is detected in this spectrum. This decision is based on the observation or non-observation of 9 peaks in 4 mass spectra, and therefore extremely robust. An analogous reasoning shows that neither G nor T can be attached to the prefix ACATGAG.

In contrast, appending the base C to the prefix ACATGAG would generate the following fragments and compomers in several different mass spectra:

| Fragment: | Compomer: | Spectra corresponding to: |
|---|---|---|
| CATGAGC | A₂C₂G₂T₁ | YRYR, RYRR, YRRR, RRRY |
| ATGAGC | A₂C₁G₂T₁ | RYRR, YRRR, RRRY |
| TGAGC | A₁C₁G₂T₁ | YRRR, RRRY |

Since all 9 peaks are observed in 4 distinct mass spectra, C is the correct character to attach. More complex cleavage patterns also can be analyzed by above method, and the robustness of the method also carries over to these complex settings.

Since modifications will be apparent to those of skill in this art, it is intended that this invention be limited only by the scope of the appended claims.

What is claimed is:

1. A method for sequencing a target nucleic acid, comprising:
a) generating overlapping fragments of a target nucleic acid;
b) contacting the fragments with an array of capture oligonucleotides under conditions that do not eliminate mismatched hybridization of the fragments to the capture oligonucleotides;
c) measuring the mass of hybridized fragments at each array locus by mass spectrometry; and
d) constructing the nucleotide sequence of the target nucleic acid from the mass measurements.

2. A method for sequencing a target nucleic acid, comprising
a) generating overlapping fragments of a target nucleic acid;
b) contacting the fragments with an array of capture oligonucleotides, wherein one or more of the capture oligonucleotides are partially degenerate;
c) measuring the mass of fragments hybridized to the capture oligonucleotides at each array position by mass spectrometry; and
d) constructing a nucleotide sequence of the target nucleic acid the mass measurements.

3. The method of claim 1, wherein the constructing step d) comprises:
tentatively constructing a nucleotide sequence containing a hypothetical nucleotide at a nucleotide locus;
predicting the fragmentation of the tentative nucleotide sequence, predicting which predicted fragments hybridize to a capture oligonucleotide, and predicting masses of hybridized predicted fragments;
comparing the predicted masses of fragments with experimentally observed masses; and
if the predicted masses match the observed masses, identifying the nucleotide locus in the target nucleic acid molecule as containing the hypothetical nucleotide.

4. The method of claim 3, wherein the step of tentatively constructing further includes tentatively constructing nucleotide sequences containing each of the four typical nucleotides at a nucleotide locus, and the predicting and comparing steps are performed for all tentative nucleotide sequences, and tentative nucleotide sequence for which the predicted masses most closely match the observed mass is identified as the nucleotide sequence in the target nucleic acid molecule.

5. The method of claim 3, wherein the tentatively constructing, predicting, comparing and identifying steps are iterated, wherein each iteration includes tentatively constructing an increasingly longer nucleotide sequence containing a hypothetical nucleotide at a nucleotide locus.

6. The method of claim 1, wherein the constructing step d) comprises:
establishing limits for fragment products of nucleic acid fragmentation;
establishing limits for nucleic acid fragments that can hybridize to a particular capture oligonucleotide;
predicting possible masses that can be observed in a mass spectrum of nucleotide fragments hybridized to the capture oligonucleotide;
comparing observed masses to the predicted masses that can be observed to identify possible sequences that could be present and/or to identify sequences that are not present; and
repeating the comparing, establishing, predicting and comparing steps for one or more additional capture oligonucleotides to thereby decrease the number of possible sequences that could be present,
whereby at least a portion of the nucleotide sequence of the target nucleic acid molecule is identified.

7. The method of claim 1, wherein the fragments are generated using a fragmentation method selected from the group consisting of enzymatic fragmentation, physical fragmentation, chemical fragmentation, and combinations thereof.

8. The method of claim 1, wherein the fragments are generated by enzymatic fragmentation using one or more enzymes, and wherein the one or more enzymes used for enzymatic fragmentation are selected from the group consisting of a non-specific RNase, a non-specific DNase, at least two double-base cutters, a preferentially-cleaving endonuclease, a restriction endonuclease, a single-base cutter, a double-base cutter, and combinations thereof.

9. The method of claim 1, wherein the fragments statistically range in a size selected from the group of size ranges consisting of 5-50 bases, 10-40 bases, 11-35 bases, and 12-30 bases.

10. The method of claim 1, wherein fewer than all theoretical combinations of capture oligonucleotide sequences are present on the array.

11. The method of claim 2, wherein the partially degenerate oligonucleotides comprise a number of degenerate positions selected from the group consisting of 1, 2, 3, 4, 5, 6, 7, 8, 9, and 10.

12. The method of claim 11, wherein each degenerate position comprises a degenerate base selected from the group consisting of a universal base and a semi-universal base.

13. The method of claim 12, wherein the universal base is selected from the group consisting of Inosine, Xanthosine, 3-nitropyrrole, 4-nitroindole, 5-nitroindole, 6-nitroindole, nitroimidazole, 4-nitropyrazole, 5-aminoindole, 4-nitrobenzimidazole, 4-aminobenzimidazole, phenyl C-ribonucleoside, benzimidazole, 5-fluoroindole, indole; acyclic sugar analogs, derivatives of hypoxanthine, imidazole 4,5-dicarboxamide, 3-nitroimidazole, 5-nitroindazole; aromatic analogs, benzene, naphthalene, phenanthrene, pyrene, pyrrole, difluorotoluene; isocarbostyril nucleoside derivatives, MICS, ICS; and hydrogen-bonding analogs, N8-pyrrolopyridine.

14. The method of claim 12, wherein the semi-universal base is selected from the group consisting of a base that hybridizes preferentially to purines A and G, a base that hybridizes to preferentially to pyrimidines C and T, a base that hybridizes to preferentially to pyrimidines C and U, 6H,8H-3,4-dihydropyrimido[4,5-c][1,2]oxazin-7-one, and N6-methoxy-2,6-diaminopurine.

15. The method of claim 1, wherein the array of capture oligonucleotides are immobilized on a solid-support selected from the group consisting of hybridization chip, pin tool, bead, polystyrene, polycarbonate, polypropylene, nylon, glass, dextran, chitin, sand, pumice, agarose, polysaccharides, dendrimers, buckyballs, polyacrylamide, silicon, metal, rubber, microtiter dish, microtiter well, glass slide, silicon chip, nitrocellulose sheet, and nylon mesh.

16. A method for controlling the complexity of a mass spectrum of target nucleic acid fragments, comprising:
(a) modulating the number of different nucleotide sequences in a first region of target nucleic acid fragments that hybridize to the capture oligonucleotide probe, whereby two or more target nucleic acid fragments containing different nucleotide sequences in the respective first regions hybridize to the capture oligonucleotide probe; and
(b) measuring the mass of the target nucleic acid fragments hybridized to the capture oligonucleotide probe by mass spectrometry,
whereby the complexity of the mass spectrum is controlled.

17. The method of claim 16, further comprising a step of controlling the length of the target nucleic acid fragments prior to measuring the mass of the target nucleic acid fragments.

18. The method of claim 16, wherein the capture oligonucleotide probe contains one or more degenerate bases.

19. The method of claim 18, wherein the degenerate bases are selected from the group consisting of universal bases and semi-universal bases.

20. The method of claim 16, wherein one or more of the target nucleic acid fragments further contain a second region that does not hybridize to the capture oligonucleotide probe.

21. The method of claim 20, wherein, of the one or more target nucleic acid fragments that contain second regions, at least two contain different nucleotide sequences in their respective second regions.

22. The method of claim 20, wherein the second regions of the one or more target nucleic acid fragments contain one or more known nucleotides at nucleotide positions at an end of the target nucleic acid fragments selected from the group consisting of the 3′ end and the 5′ end.

23. The method of claim 16, wherein the step of controlling the length of target nucleic acid fragments further includes base-specific cleavage.

24. The method of claim 16, wherein the target nucleic acid fragments are hybridized to an array of capture oligonucleotide probes, wherein the array contains a plurality of positions, and the nucleotide sequence of the capture oligonucleotide probes at each array position differs from the nucleotide sequence of capture oligonucleotide probes at all other array positions.

25. A method of identifying a portion of a target nucleic acid, comprising:
(a) collecting a mass spectrum with controlled complexity according to the method of claim 16; and
(b) comparing the one or more target nucleic acid fragment masses with one or more masses of one or more reference nucleic acids,
wherein a correlation between one or more target nucleic acid fragment masses and one or more reference masses identifies a portion of the target nucleic acid as corresponding to the reference nucleic acid or corresponding to a portion of the reference nucleic acid.

26. The method of claim 25, wherein the one or more reference masses of at least one reference nucleic acid are calculated.

27. The method of claim 25, wherein the one or more reference masses of at least one reference nucleic acid are experimentally measured.

28. The method of claims 25, wherein the target nucleic acid fragments are formed using a method selected from sequence-specific fragmentation and non-specific fragmentation.

29. The method of claim 25, wherein the portion of the target nucleic acid identified contains a SNP.

30. A composition for identifying a portion of a target nucleic acid, comprising:
(a) an array of two or more capture oligonucleotides on a solid support, wherein at least one capture oligonucleotide is partially degenerate; and
(b) a mass spectrometer operably coupled to the array.

31. The composition of claim 30, further comprising a computer program for constructing a nucleotide sequence of the target nucleic acid from a set of mass signals acquired from nucleic acid molecules that hybridize to the capture oligonucleotides.
